(19) United States
(12) Patent Application Publication
(10) Pub. No.: US 2018/0099974 A1
(43) Pub. Date: Apr. 12, 2018

(54) METHODS OF TREATING OR PREVENTING A PROTEOPATHY

(71) Applicant: Usher III Initiative, Inc., Chicago, IL (US)

(72) Inventor: David Saperstein, Seattle, WA (US)

(21) Appl. No.: 15/835,624

(22) Filed: Dec. 8, 2017

Related U.S. Application Data
(63) Continuation of application No. PCT/US2016/036945, filed on Jun. 10, 2016.
(60) Provisional application No. 62/174,332, filed on Jun. 11, 2015.
(60) Provisional application No. 62/174,338, filed on Jun. 11, 2015.

(51) Int. Cl. C07D 487/04; A61P 25/28

(52) U.S. Cl. CPC C07D 487/04; A61P 25/28

(57) ABSTRACT

Methods for the treatment or prevention of a proteopathy are described herein.

### CROSS REFERENCE TO RELATED APPLICATIONS

This application claims the benefit of U.S. provisional application No. 62/174,332, filed Jun. 11, 2015, and U.S. provisional application No. 62/174,338, filed Jun. 11, 2015, each of which is incorporated by reference herein in its entirety.

### BACKGROUND OF THE INVENTION

The proper functioning of organs and cells within an organism relies on the proper function of proteins. A protein is a biological entity that has a primary amino acid sequence; a secondary structure that forms protein domains and includes, most importantly, alpha helices and beta sheets; and a tertiary structure, a result of a complex folding of the peptide chain in three dimensions that involve the polypeptide chain backbone and amino acid side chain interactions. Some proteins work in a multi-subunit complex, where the arrangement of multiple proteins into a quaternary structure becomes crucial for their proper function.

The failure of proteins to fold into correct three-dimensional structures can lead to diseases called proteopathies (sometimes also referred to as proteinopathies or protein conformational disorders). The failure may be due to one or more mutations in the proteins' gene or to environmental factors such as oxidative stress, alkalosis, acidosis, pH shift and osmotic shock. The misfolding of proteins can sometimes lead to clumping or aggregation into amyloid plaques or fibrils that can exacerbate a disease. Proteopathies cover a wide spectrum of afflictions, including neurodegenerative diseases (e.g., Alzheimer's, Parkinson's, polyglutamine diseases, prion diseases); amyloidosis of other non-nervous system proteins such as α1-antitrypsin, immunoglobulin light and heavy chains, lactadherin, apolipoprotein, gelsolin, lysozyme, fibrinogen, atrial natriuretic factor, keratin, lactoferrin and beta-2 microglobulin, among others); sickle cell disease; cataracts; cystic fibrosis; retinitis pigmentosa; and nephrogenic diabetes insipidus.

Molecular chaperones are biological molecules that assist in proper protein folding, protein translocation, and/or protein degradation. Examples of molecular chaperones include the heat shock proteins, which are classified into seven different families in the human genome and include HSPH (Hsp110), HSPC (Hsp90), HSPA (Hsp70), DNAJ (Hsp40), HSPB (small Hsp (sHsp)), the human chaperonins HSPD/E (HSP60/HSP10) and CCT (TRiC).

### SUMMARY OF THE INVENTION

The invention provides methods for treating or preventing a proteopathy, comprising administering to a subject in need thereof an effective amount of a compound of Formula I:

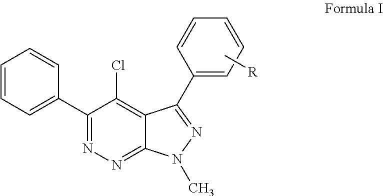

or a pharmaceutically acceptable salt thereof,
wherein R is fluoro, chloro, iodo, methyl, methoxy, cyano, trifluoromethyl, or —(CO)NH(CH₃).

The invention also provides methods for treating or preventing a proteopathy, comprising administering to a subject in need thereof an effective amount of a compound of Formula II:

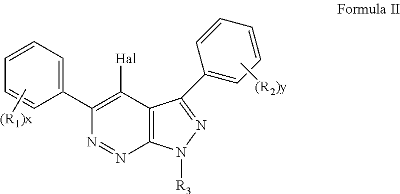

or a pharmaceutically acceptable salt thereof,
wherein Hal is —Cl, —F, —I, or —Br;
x is an integer ranging from 0 to 5;
each R₁ is independently —Cl, —F, —I, —Br, —C₁-C₃ alkyl, —O—C₁-C₃ alkyl, —CN, —CF₃, —C(O)NH(CH₃), or —C≡CCH₂OH;
y is an integer ranging from 0 to 5;
each R₂ is independently —Cl, —F, —Br, —C₁-C₃ alkyl, —O—C₁-C₃ alkyl, —CN, —CF₃, —C(O)NH(CH₃), or —C≡CCH₂OH;
R₃ is —H, —C₁-C₆ alkyl, —(C₁-C₆ alkylene)-OH, —(C₁-C₆ alkylene)-phenyl, —(C₁-C₆ alkylene)-O—(C₁-C₆ alkyl), —C₂-C₆ alkenyl, —(C₁-C₆ alkylene)-C(O)R₄, —(C₁-C₆ alkylene)-R₅,

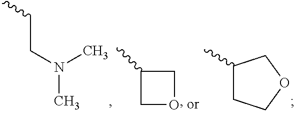

R₄ is —OH, —O—(C₁-C₆ alkyl), —NH₂, —NH(C₁-C₆ alkyl), —NH((C₁-C₆ alkylene)-OH), —NH((C₁-C₆ alkylene)N(C₁-C₆ alkyl)₂), —N(C₁-C₆ alkyl)((C₁-C₆ alkylene)-CN), —N(C₁-C₆ alkyl)((C₁-C₆ alkylene)N(C₁-C₆ alkyl)₂), —NH(C₁-C₆ alkylene)-O—(C₁-C₆

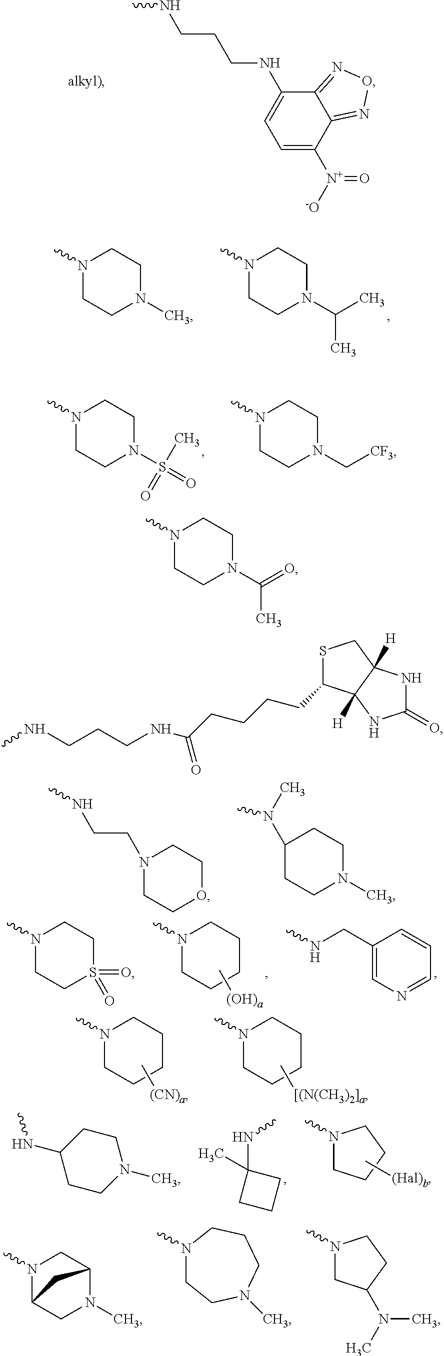

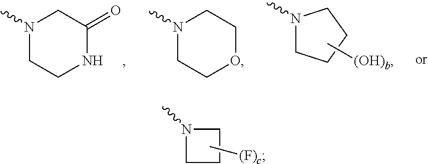

a is an integer ranging from 0 to 10;
b is an integer ranging from 0 to 8;
c is an integer ranging from 0 to 6; and
R₅ is

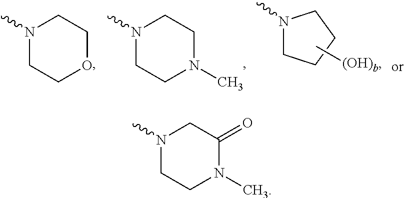

The invention further provides methods for treating or preventing a proteopathy, comprising administering to a subject in need thereof an effective amount of a compound of Formula III:

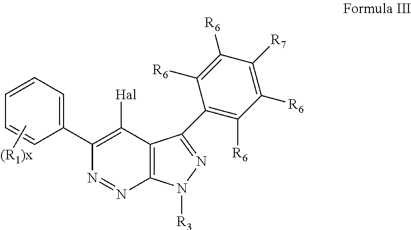

or a pharmaceutically acceptable salt thereof,
wherein Hal is —Cl, —F, —I, or —Br;
x is an integer ranging from 0 to 5;
each R₁ is independently —Cl, —F, —I, —Br, —C₁-C₃ alkyl, —O—C₁-C₃ alkyl, —CN, —CF₃, —C(O)NH(CH₃), or —C≡CCH₂OH;
R₃ is —H, —C₁-C₆ alkyl, —(C₁-C₆ alkylene)-OH, —(C₁-C₆ alkylene)-phenyl, —(C₁-C₆ alkylene)-O—(C₁-C₆ alkyl), —C₂-C₆ alkenyl, —(C₁-C₆ alkylene)-C(O)R₄, —(C₁-C₆ alkylene)-R₅,

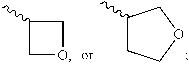

R₄ is —OH, —O—(C₁-C₆ alkyl), —NH₂, —NH(C₁-C₆ alkyl), —NH((C₁-C₆ alkylene)-OH), —NH((C₁-C₆ alkylene)N(C₁-C₆ alkyl)₂), —N(C₁-C₆ alkyl)((C₁-C₆ alkylene)-CN), —N(C₁-C₆ alkyl)((C₁-C₆ alkylene)N(C₁-C₆ alkyl)₂), —NH(C₁-C₆ alkylene)-O—(C₁-C₆ alkyl),

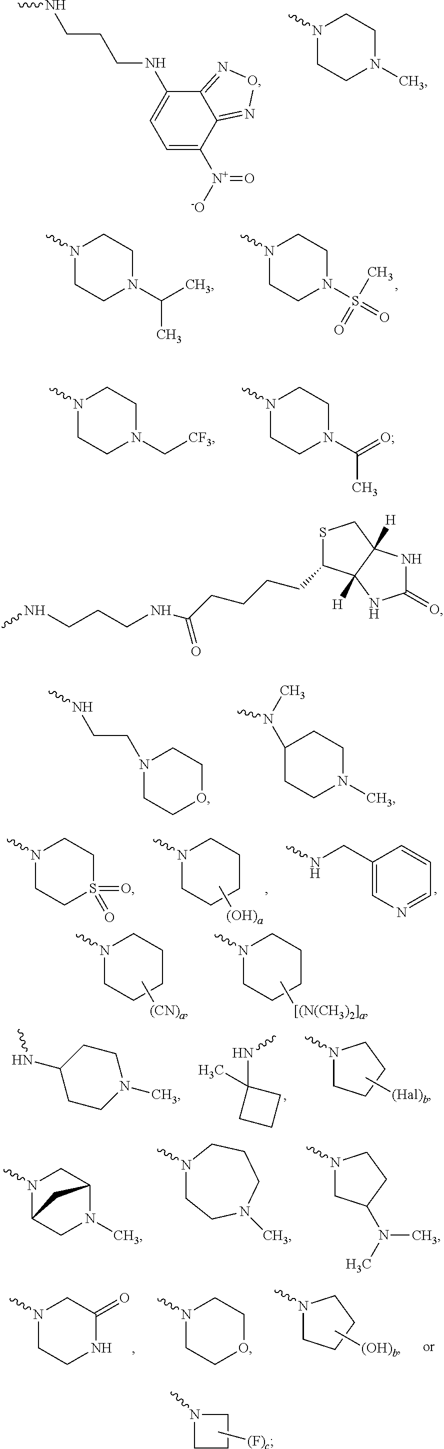

a is an integer ranging from 0 to 10;
b is an integer ranging from 0 to 8;
c is an integer ranging from 0 to 6;
R₅ is

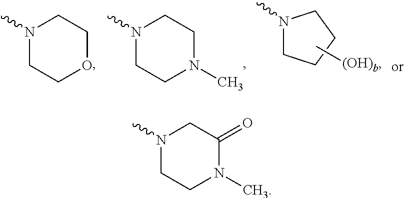

and
each R₆ and R₇ is independently —H or —I, wherein at least one of R₆ and R₇ is —I,
and wherein when R₃ is —C₁-C₃ alkyl, R₇ is —H.

The invention further provides methods for treating or preventing a proteopathy, comprising administering to a subject in need thereof an effective amount of a compound of Formula IV:

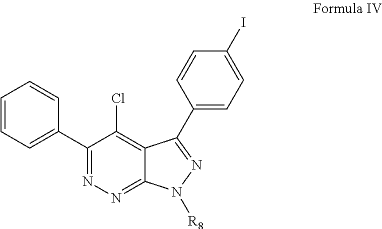

or a pharmaceutically effective salt thereof,
wherein R₈ is —C₁-C₃ alkyl.

The invention further provides methods for treating or preventing a proteopathy, comprising administering to a subject in need thereof an effective amount of a compound having the structure:

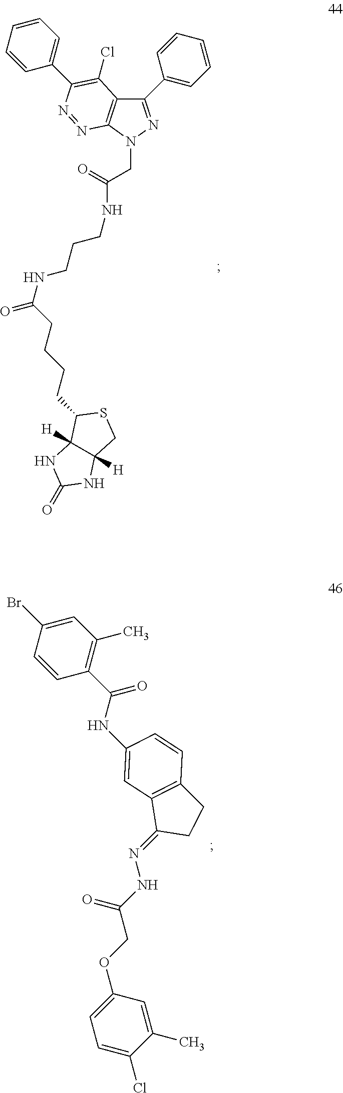 [SMILES: O=C(CCCC[C@@H]1SC[C@@H]2NC(=O)N[C@@H]21)NCCCNC(=O)Cn1nc(-c2ccccc2)c2c(Cl)c(-c3ccccc3)nnc21 ; Cc1cc(OCC(=O)N/N=C2\CCc3ccc(NC(=O)c4ccc(Br)cc4C)cc32)ccc1Cl]

or a pharmaceutically acceptable salt thereof.

The invention still further provides methods for treating or preventing a proteopathy, comprising administering to a subject in need thereof an effective amount of a compound having the structure:

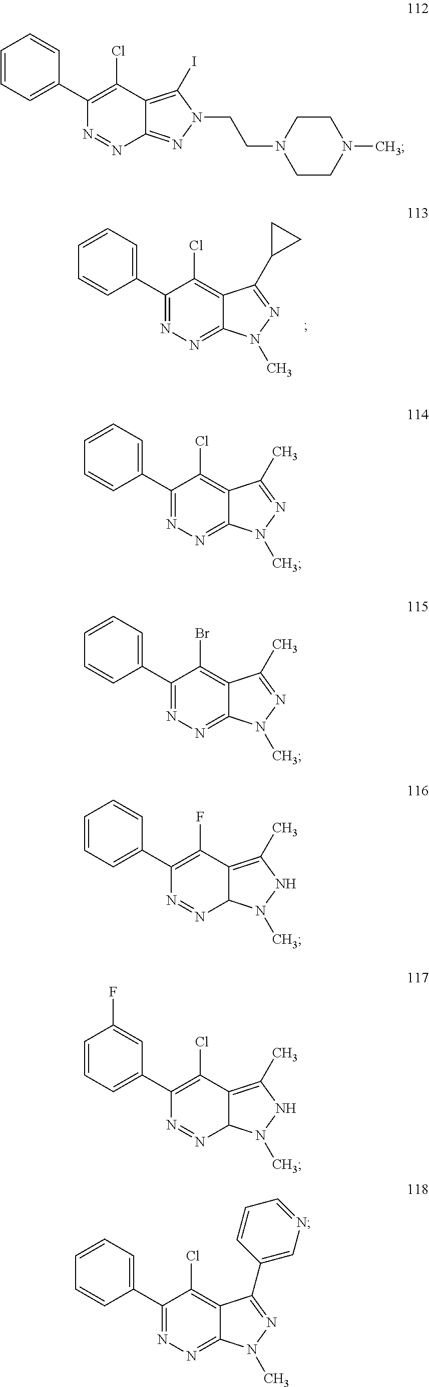

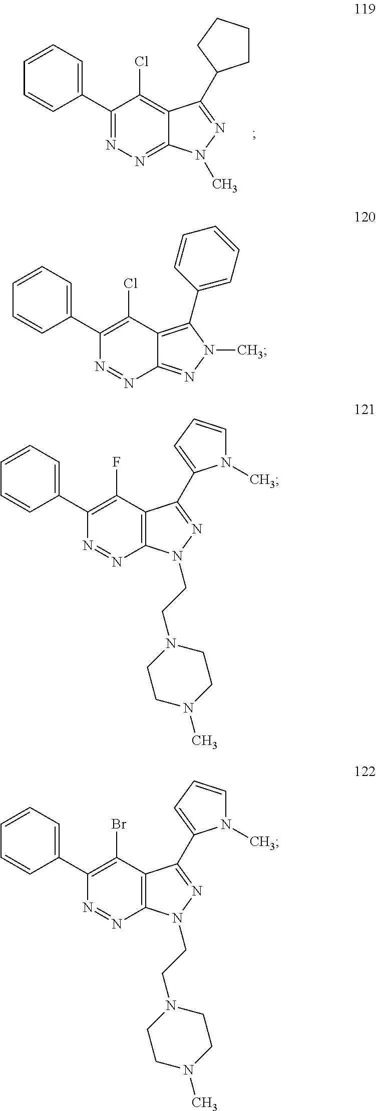

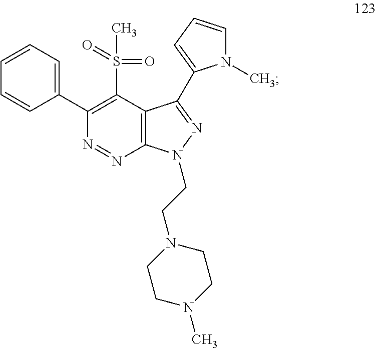 [SMILES: CN1CCN(CCn2nc(-c3cccn3C)c3c(S(C)(=O)=O)c(-c4ccccc4)nnc32)CC1]

or a pharmaceutically acceptable salt thereof.

The invention still further provides methods for treating or preventing a proteopathy, comprising administering to a subject in need thereof an effective amount of a compound of Formula V:

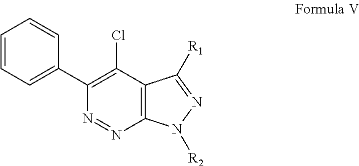

or a pharmaceutically acceptable salt thereof,
wherein R₁ is:

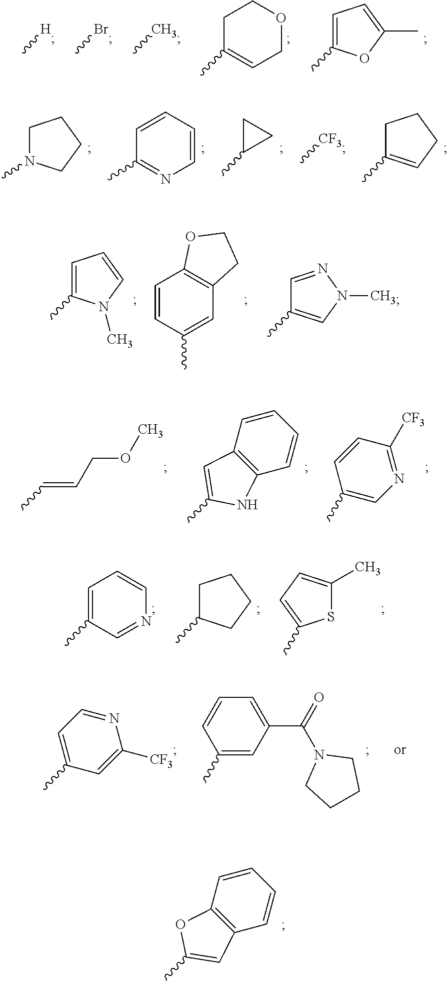

R₂ is:

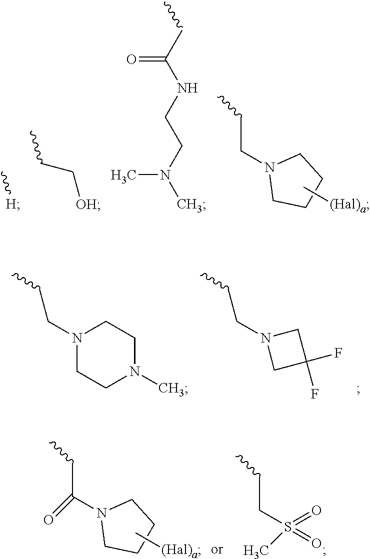

Hal is —Cl, —F, —I, or —Br; and
and a is 0, 1, or 2.

The invention still further provides methods for treating or preventing a proteopathy, comprising administering to a subject in need thereof an effective amount of a compound of Formula VI:

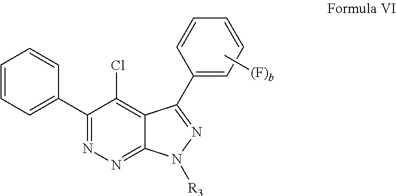

or a pharmaceutically acceptable salt thereof,
wherein R₃ is:

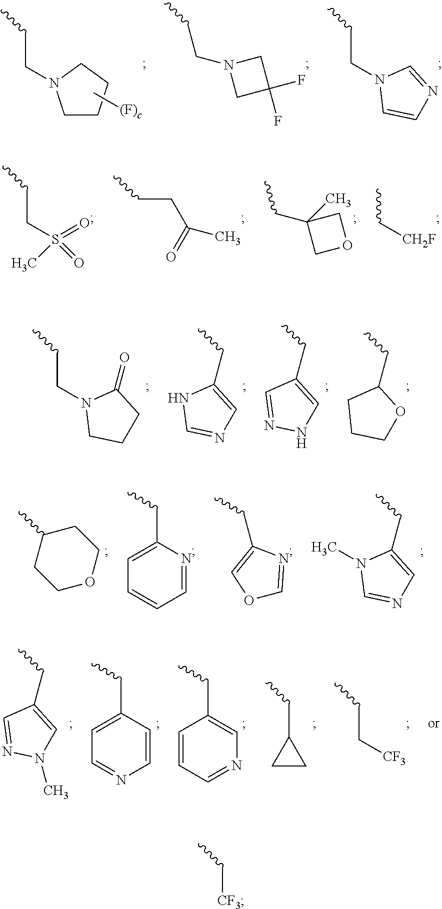

b is 0 or 1; and
c is 1 or 2.

The invention still further provides methods for treating or preventing a proteopathy, comprising administering to a subject in need thereof an effective amount of a compound of Formula VII:

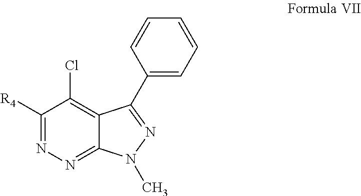

or a pharmaceutically acceptable salt thereof,
wherein R₄ is

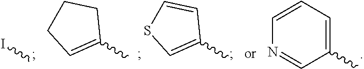

The invention still further provides methods for treating or preventing a proteopathy, comprising administering to a subject in need thereof an effective amount of a compound of Formula XIII:

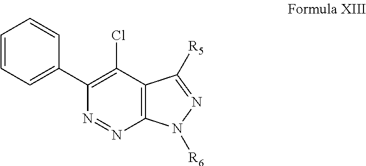

or a pharmaceutically acceptable salt thereof,
wherein R₅ is:

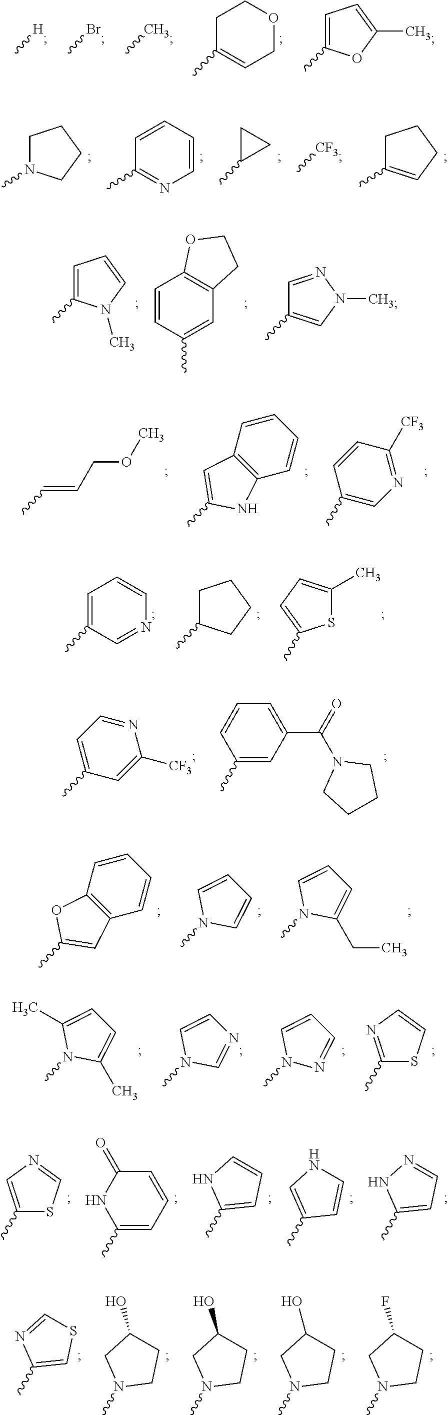

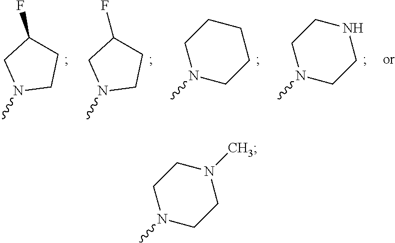

R₆ is:

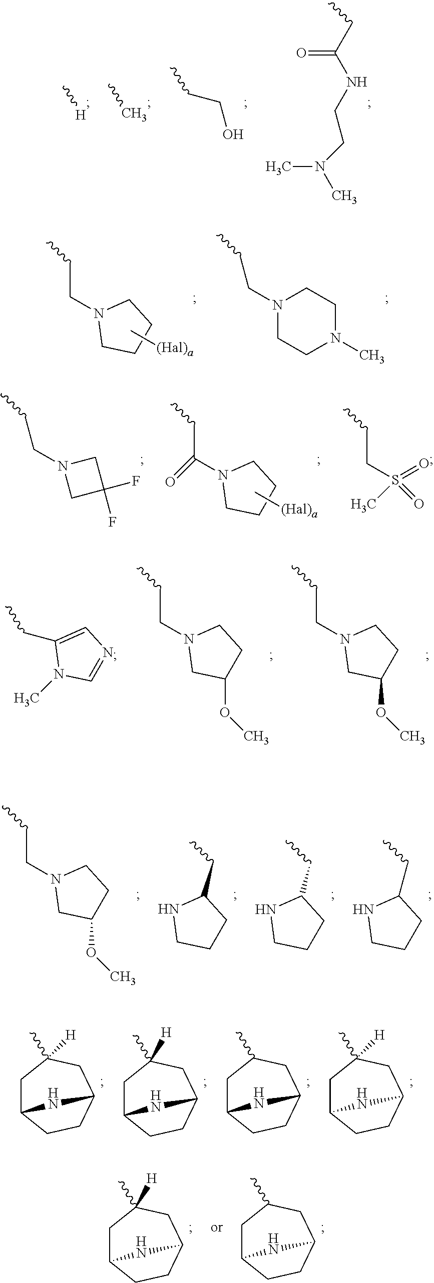

Hal is —Cl, —F, —I, or —Br; and
a is 0, 1, or 2.

The invention still further provides methods for treating or preventing a proteopathy, comprising administering to a subject in need thereof an effective amount of a compound of Formula XIV:

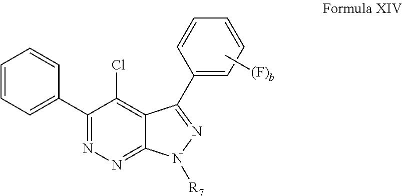

or a pharmaceutically acceptable salt thereof,
wherein R₇ is:

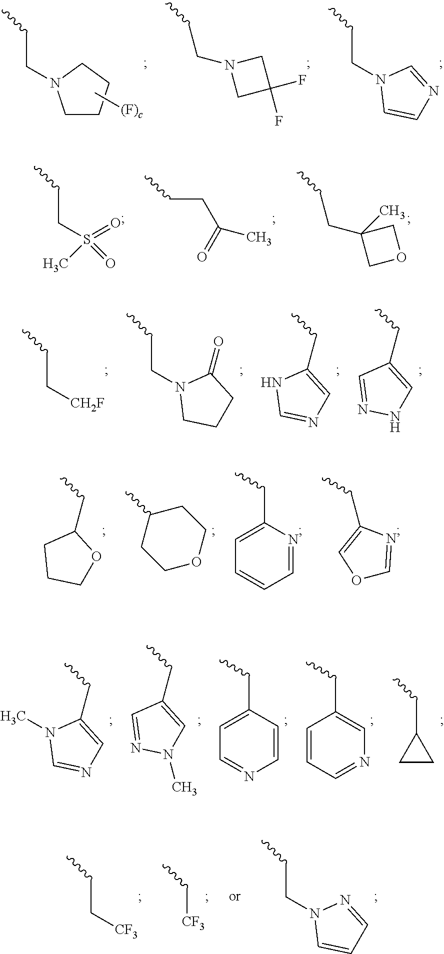

b is 0 or 1; and
c is 1 or 2.

The invention still further provides methods for treating or preventing a proteopathy, comprising administering to a subject in need thereof an effective amount of a compound of Formula XV:

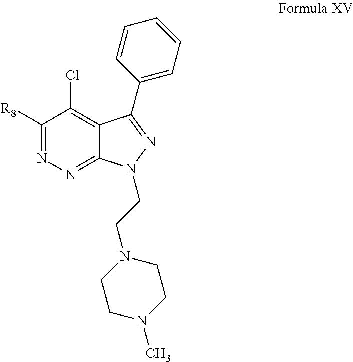

or a pharmaceutically acceptable salt thereof,
wherein R₈ is:

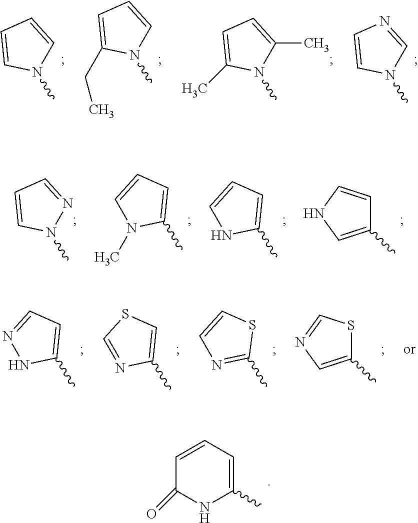

A “Pyrazolopyridazine compound” is: a compound of Formula I, II, III, IV, V, VI, VII, XIII, XIV or XV; Compound 1-35, 37-39, 42, 43, 44, 45, 46, 47-97, 98-123, 124a, 124b; or a pharmaceutically acceptable salt of any of the foregoing. A Pyrazolopyridazine compound is useful for treating or preventing a proteopathy.

### DETAILED DESCRIPTION OF THE INVENTION

In one embodiment, the invention provides Pyrazolopyridazine compounds. In a further embodiment, the invention provides pharmaceutical compositions comprising an effective amount of a Pyrazolopyridazine compound and a pharmaceutically acceptable carrier or vehicle.

In still a further embodiment, the invention provides methods for treating or preventing a proteopathy, comprising administering to a subject in need thereof an effective amount of a Pyrazolopyridazine compound.

### Definitions

The term “alkyl” refers to a straight or branched saturated hydrocarbon group. Illustrative alkyl groups include —CH₃, —CH₂CH₃, —CH₂CH₂CH₃, —CH(CH₃)₂, —CH₂CH₂CH₂CH₃, —CH(CH₃)CH₂CH₃, —CH₂CH(CH₃)₂, —C(CH₃)₃, —CH₂CH₂CH₂CH₂CH₃, —CH(CH₃)CH₂CH₂CH₃, —CH₂CH₂CH(CH₃)₂, —CH₂C(CH₃)₃, —CH₂CH₂CH₂CH₂CH₃, —CH(CH₃)CH₂CH₂CH₃, —CH₂CH₂CH(CH₃)₂ and —CH(CH₃)C(CH₃)₃ groups.

The term “alkylene” refers to an alkyl group bonded to another atom or group. Illustrative alkylene groups include —CH₂—, —CH₂CH₂—, —CH₂CH₂CH₂—, —C(CH₃)₂—, —CH(CH₃), —CH₂CH₂CH₂CH₂—, —CH(CH₃)CH₂CH₂—, —CH₂C(CH₃)₂—, —C(CH₃)₂CH₂—, —CH₂CH₂CH₂CH₂CH₂—, —CH(CH₃)CH₂CH₂CH₂—, —CH₂CH₂C(CH₃)₂—, —CH₂CH(CH₃)CH₂CH₂, —CH₂CH₂CH(CH₃)CH₂—, —CH₂CH₂CH₂CH₂CH₂CH₂—, —CH(CH₃)CH₂CH₂CH₂CH₂—, —CH₂CH₂CH₂C(CH₃)₂—, —CH₂CH(CH₃)CH₂CH₂CH₂—, —CH₂CH₂CH₂CH(CH₃)CH₂— and —C(CH₃)₂C(CH₃)₂— groups.

The term “alkenyl” refers to a straight or branched hydrocarbon group having one or more double bonds. Illustrative alkenyl groups include —CH═CH₂, —CH₂CH═CH₂, cis —CH═CHCH₃, trans —CH═CHCH₃, —C(CH₃)═CH₂, cis —CH═CHCH₂CH₃, trans —CH═CHCH₂CH₃, cis —CH₂CH═CHCH₃, trans —CH₂CH═CHCH₃, —CH₂CH₂CH═CH₂, cis —CH═CHCH₂CH₂CH₃, trans —CH═CHCH₂CH₂CH₃, cis —CH₂CH₂CH═CHCH₃, trans —CH₂CH₂CH═CHCH₃, —CH₂CH₂CH₂CH═CH₂, —CH₂CH═C(CH₃)₂, cis —CH═CHCH₂CH₂CH₂CH₃, trans —CH═CHCH₂CH₂CH₂CH₃, cis —CH₂CH₂CH₂CH═CHCH₃, trans —CH₂CH₂CH₂CH═CHCH₃, —CH₂CH₂CH₂CH₂CH═CH₂, and —CH₂CH₂CH═C(CH₃)₂, groups.

The word “about” when immediately preceding a numerical value means a range of plus or minus 10% of that value, e.g., “about 100 mg” means 90 mg to 110 mg, “about 300 mg” means 270 mg to 330 mg, etc.

Abbreviations:
APCI Atmospheric Pressure Chemical Ionization
DAPI 4′,6-diamidino-2-phenylindole
DCM dichloromethane
DEAD diethyl azodicarboxylate
DIPEA diisopropylethylamine
DMEM Dulbecco's Modified Eagle Medium
DMF dimethylformamide
DMSO Dimethyl sulfoxide
EDAC 1-(3-dimethylaminopropyl)-3-ethylcarbodiimide hydrochloride
ESI Electrospray ionization
ESI-TOF Electrospray ionization-Time-of-flight
HATU 2-(7-Aza-1H-benzotriazole-1-yl)-1,1,3,3-tetramethyluronium hexafluorophosphate
HOPO 2-hydroxypyridine-N-oxide
HPLC High-performance liquid chromatography
LCMS Liquid chromatography-mass spectrometry
LDA lithium diisopropyl amide
m/z Mass-to-charge ratio
MALDI-TOF Matrix Assisted Laser Desorption Ionization-Time-of-flight
MS Mass spectrometry
PBS phosphate-buffered saline
Rt Retention time
SDS sodium dodecylsulfate
TFA trifluoroacetic acid
THF tetrahydrofuran

The term “effective amount” means an amount of a Pyrazolopyridazine compound or non-Pyrazolopyridazine compound that is effective to treat or prevent a proteopathy in a subject. In some embodiments, where another therapeutic or prophylactic agent is administered prior to, subsequent to or concurrently with administration of a Pyrazolopyridazine compound or non-Pyrazolopyridazine compound, the “effective amount” is the total amount of (i) Pyrazolopyridazine compound or non-Pyrazolopyridazine compound and (ii) the other therapeutic or prophylactic agent that is effective to treat or prevent a protopathy in a subject.

The terms “proteopathy”, “proteinopathy” and “protein conformational disorder” refer to a disease or a disorder resulting from the misfolding of one or more proteins.

The term “protein aggregate” refers to a biological phenomenon in which misfolded proteins accumulate and clump together.

A “subject” is a mammal, including a species-rich order, e.g., a primate, such as a human; a Rodentia species, such as a mouse, a rat or a guinea pig; a Carnivora species such as a dog, cat, weasel, bear or seal; a non-human primate, such as a monkey, chimpanzee, baboon or rhesus; a Chiroptera species, such as a bat; a Soricomorpha species, such as a shrew, mole or solenodon; and a Cetartiodactyla species, such as a whale. In one embodiment, the subject is a human. In another embodiment, the human is a human fetus.

### Pyrazolopyridazine Compounds Useful in the Present Methods

Pyrazolopyridazine Compounds of Formula I

In one embodiment, the invention provides methods for treating or preventing a proteopathy, comprising administering to a subject in need thereof an effective amount of a compound of Formula I:

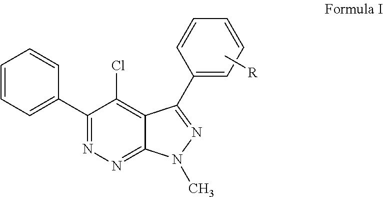

or a pharmaceutically acceptable salt thereof,
wherein R is fluoro, chloro, iodo, methyl, methoxy, cyano, trifluoromethyl, or —(CO)NH(CH₃).

In one embodiment, R of Formula I is in the para position relative to the pyrazolopyridazino ring system. In one embodiment, R of Formula I is in the meta position relative to the pyrazolopyridazino ring system. In one embodiment, R of Formula I is in the ortho position relative to the pyrazolopyridazino ring system.

Pyrazolopyridazine Compounds of Formula II

The invention also provides methods for treating or preventing a proteopathy, comprising administering to a subject in need thereof an effective amount of a compound of Formula II:

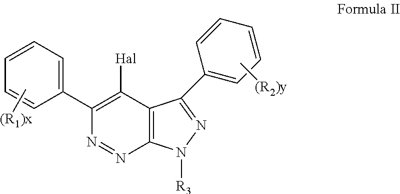

or a pharmaceutically acceptable salt thereof,
wherein Hal is —Cl, —F, —I, or —Br;
x is an integer ranging from 0 to 5;
each R₁ is independently —Cl, —F, —I, —Br, —C₁-C₃ alkyl, —O—C₁-C₃ alkyl, —CN, —CF₃, —C(O)NH(CH₃), or —C≡CCH₂OH;
y is an integer ranging from 0 to 5;
each R₂ is independently —Cl, —F, —Br, —C₁-C₃ alkyl, —O—C₁-C₃ alkyl, —CN, —CF₃, —C(O)NH(CH₃), or —C≡CCH₂OH;
R₃ is —H, —C₁-C₆ alkyl, —(C₁-C₆ alkylene)-OH, —(C₁-C₆ alkylene)-phenyl, —(C₁-C₆ alkylene)-O—(C₁-C₆ alkyl), —C₂-C₆ alkenyl, —(C₁-C₆ alkylene)-C(O)R₄, —(C₁-C₆ alkylene)-R₅,

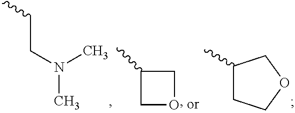

R₄ is —OH, —O—(C₁-C₆ alkyl), —NH₂, —NH(C₁-C₆ alkyl), —NH((C₁-C₆ alkylene)-OH), —NH((C₁-C₆ alkylene)N(C₁-C₆ alkyl)₂), —N(C₁-C₆ alkyl)((C₁-C₆ alkylene)-CN), —N(C₁-C₆ alkyl)((C₁-C₆ alkylene)N(C₁-C₆ alkyl)₂), —NH(C₁-C₆ alkylene)-O—(C₁-C₆ alkyl),

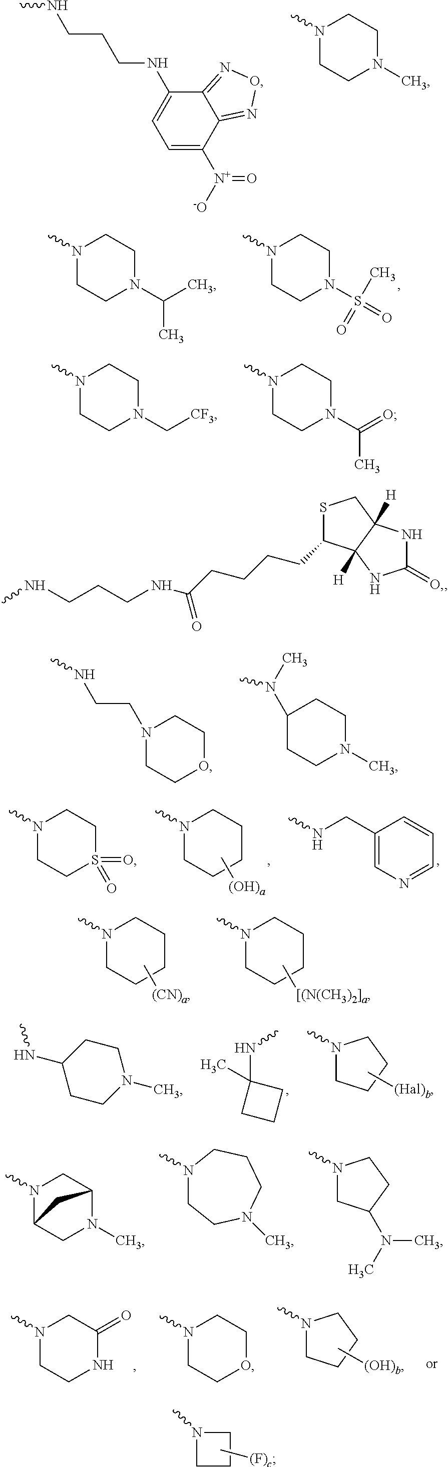

a is an integer ranging from 0 to 10;
b is an integer ranging from 0 to 8;
c is an integer ranging from 0 to 6; and
R₅ is

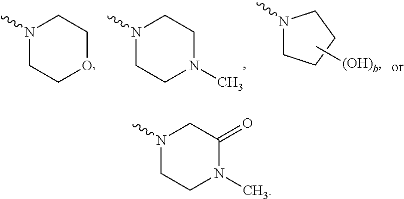

In certain embodiments, Hal is —Cl. In yet another embodiment, x and y are 0.

In certain embodiments, x and y are 0, x is 0 and y is 1, x is 1 and y is 2, x is 1 and y is 0, x is 1 and y is 1, x is 1 and y is 2, x is 2 and y is 0, x is 2 and y is 1, or x is 2 and y is 2.

In certain embodiments, Hal is —Cl and: x and y are 0, x is 0 and y is 1, x is 1 and y is 2, x is 1 and y is 0, x is 1 and y is 1, x is 1 and y is 2, x is 2 and y is 0, x is 2 and y is 1, or x is 2 and y is 2.

In particular embodiments, x is 1 and R₁ is in the ortho position relative to the pyrazolopyridazino ring system. In certain embodiments, x is 1 and R₁ is in the para position relative pyrazolopyridazino ring system. In further embodiments, x is 1 and R₁ is in the meta position relative pyrazolopyridazino ring system.

In particular embodiments, y is 1 and R₂ is in the ortho position relative pyrazolopyridazino ring system. In certain embodiments, y is 1 and R₂ is in the para position relative pyrazolopyridazino ring system. In further embodiments, y is 1 and R₂ is in the meta position relative pyrazolopyridazino ring system.

In particular embodiments, x is 2 and R₁ is in the ortho and meta position relative pyrazolopyridazino ring system. In certain embodiments, x is 2 and R₁ is in the ortho and para position relative pyrazolopyridazino ring system. In further embodiments, x is 2 and R₁ is in the para and meta position relative pyrazolopyridazino ring system.

In particular embodiments, y is 2 and R₂ is in the ortho and meta position relative pyrazolopyridazino ring system. In certain embodiments, y is 2 and R₂ is in the ortho and para position relative pyrazolopyridazino ring system. In further embodiments, y is 2 and R₂ is in the para and meta position relative pyrazolopyridazino ring system.

In yet other embodiments, R₁ is chloro. In certain embodiments, R₁ is fluoro. In certain embodiments, R₁ is iodo. In other embodiments, R₁ is —Br. In further embodiments, R₁ is —OCH₃. In other embodiments, R₁ is —CH₃. In yet other embodiments, R₁ is —C(O)N(H)CH₃. In certain embodiments, R₁ is —CF₃. In further embodiments, R₁ is —CN. In additional embodiments, R₁ is —C≡CCH₂OH.

In yet other embodiments, x is 1 or 2, and R₁ is —Cl, —F, —I, —Br, —OCH₃, —CH₃, —C(O)N(H)CH₃, —CF₃, —CN or —C≡CCH₂OH.

In yet other embodiments, Hal is —Cl, x is 1 or 2, and R₁ is —Cl, —F, —I, —Br, —OCH₃, —CH₃, —C(O)N(H)CH₃, —CF₃, —CN or —C≡CCH₂OH.

In yet other embodiments, R₂ is —Cl. In certain embodiments, R₂ is —F. In other embodiments, R₂ is —Br. In further embodiments, R₂ is —OCH₃. In other embodiments, R₂ is —CH₃. In yet other embodiments, R₂ is —C(O)N(H)CH₃. In certain embodiments, R₂ is —CF₃. In further embodiments, R₂ is —CN. In additional embodiments, R₂ is —C≡CCH₂OH.

In yet other embodiments, y is 1 or 2, and R₂ is —Cl, —F, —Br, —OCH₃, —CH₃, —C(O)N(H)CH₃, —CF₃, —CN or —C≡CCH₂OH.

In yet other embodiments, Hal is —Cl, y is 1 or 2, and R₂ is —Cl, —F, —Br, —OCH₃, —CH₃, —C(O)N(H)CH₃, —CF₃, —CN or —C≡CCH₂OH.

In particular embodiments, R₃ is —H. In certain embodiments, R₃ is —CH₃. In further embodiments, R₃ is —CH₂CH₃. In still further embodiments, R₃ is —CHCH₂. In other embodiments, R₃ is —CH₂CH₂OH. In particular embodiments, R₃ is —(CH₂)₂C₆H₅. In other embodiments, R₃ is —CH₂C(O)OH. In yet other embodiments, R₃ is —CH₂C(O)N(H)CH₃. In certain embodiments, R₃ is —CH₂C(O)N(H)((CH₂)₂N(CH₃)₂).
In yet other embodiments, R₃ is —CH₂C(O)N(H)((CH₂)₃N(CH₃)₂). In other embodiments, R₃ is —CH₂C(O)N(CH₃)CH₂CN. In particular embodiments, R₃ is —CH₂C(O)NH₂. In certain embodiments, R₃ is —CH₂C(O)N(H)((CH₂)₂OH). In other embodiments, R₃ is —CH₂C(O)N(H)((CH₂)₂OCH₃). In still further embodiments, R₃ is —CH₂C(CH₃)₂OH.
In yet other embodiments, R₃ is —CH₂C(O)OCH₃. In further embodiments, R₃ is —CH₂CH(OH)CH₃. In still further embodiments, R₃ is —CH₂CH₂OH. In particular embodiments,
R₃ is —CH(CH₃)CH₂OH.

In further embodiments, R₃ is —CH₂C(O)R₄ and R₄ is

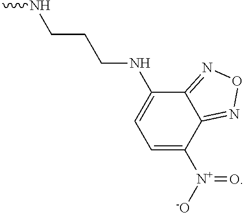

In other embodiments, R₃ is —CH₂C(O)R₄ and R₄ is

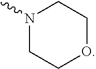

In particular embodiments, R₃ is —CH₂C(O)R₄ and R₄ is

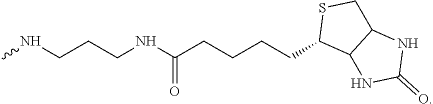

In yet other embodiments, R₃ is —CH₂C(O)R₄ and R₄ is

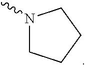

In certain embodiments, R₃ is —CH₂C(O)R₄ and R₄ is

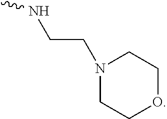

In other embodiments, R₃ is —CH₂C(O)R₄ and R₄ is

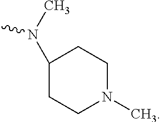

In particular embodiments, R₃ is —CH₂C(O)R₄ and R₄ is

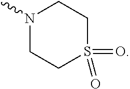

In yet other embodiments, R₃ is —CH₂C(O)R₄ and R₄ is

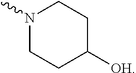

In certain embodiments, R₃ is —CH₂C(O)R₄ and R₄ is

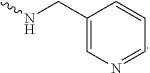

In other embodiments, R₃ is —CH₂C(O)R₄ and R₄ is

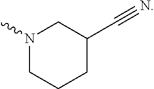

In particular embodiments, R₃ is —CH₂C(O)R₄ and R₄ is

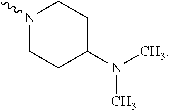

In yet other embodiments,
R₃ is —CH₂C(O)R₄ and R₄ is

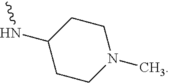

In certain embodiments, R₃ is —CH₂C(O)R₄ and R₄ is

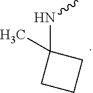

In other embodiments, R₃ is —CH₂C(O)R₄ and R₄ is

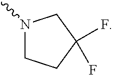

In particular embodiments, R₃ is —CH₂C(O)R₄ and R₄ is

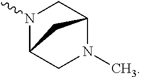

In yet other embodiments, R₃ is —CH₂C(O)R₄ and R₄ is

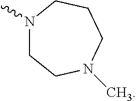

In certain embodiments, R₃ is —CH₂C(O)R₄ and R₄ is

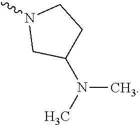

In other embodiments, R₃ is —CH₂C(O)R₄ and R₄ is

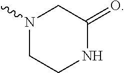

In particular embodiments, R₃ is —CH₂C(O)R₄ and R₄ is

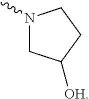

In yet other embodiments, R₃ is —CH₂C(O)R₄ and R₄ is

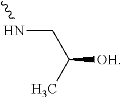

In certain embodiments, R₃ is —CH₂C(O)R₄ and R₄ is

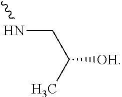

In other embodiments, R₃ is —CH₂C(O)R₄ and R₄ is

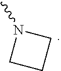

In particular embodiments, R₃ is —CH₂C(O)R₄ and R₄ is

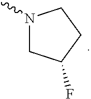

In yet other embodiments, R₃ is —CH₂C(O)R₄ and R₄ is

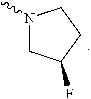

In certain embodiments, R₃ is —CH₂C(O)R₄ and R₄ is

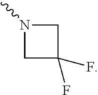

In other embodiments, R₃ is —CH₂C(O)R₄ and R₄ is

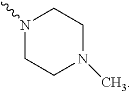

In further embodiments of the invention, R₃ is —CH₂C(O)R₄ and R₄ is

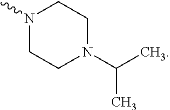

In certain embodiments of the invention, R₃ is —CH₂C(O)R₄ and R₄ is

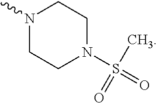

In other embodiments, R₃ is —CH₂C(O)R₄ and R₄ is

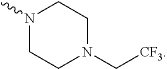

In further embodiments, R₃ is —CH₂C(O)R₄ and R₄ is

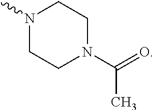

In further embodiments, R₃ is —CH₂C(O)R₄ and R₄ is

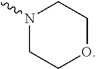

In particular embodiments, R₃ is —(CH₂)₂R₅ and R₅ is

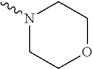

In yet other embodiments, R₃ is —(CH₂)₂R₅ and R₅ is

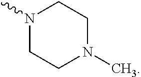

In certain embodiments, R₃ is —(CH₂)₂R₅ and R₅ is

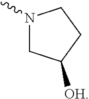

In other embodiments, invention, R₃ is —(CH₂)₂R₅ and R₅ is

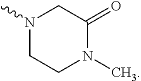

In some embodiments, a is an integer ranging from 0 to 5. In some embodiments, b is an integer ranging from 0 to 4. In some embodiments, c is an integer ranging from 0 to 6.

Illustrative Pyrazolopyridazine Compounds of Formula II

In certain embodiments the Pyrazolopyridazine compound of Formula II has the structure:

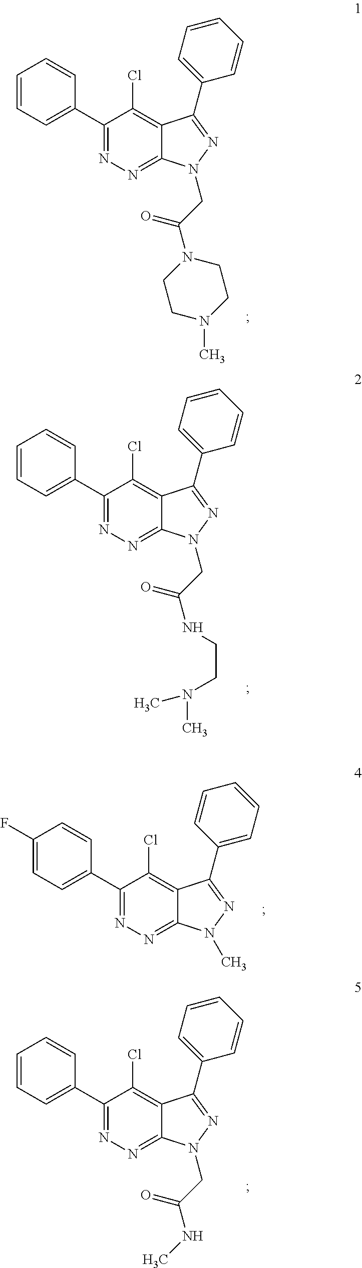

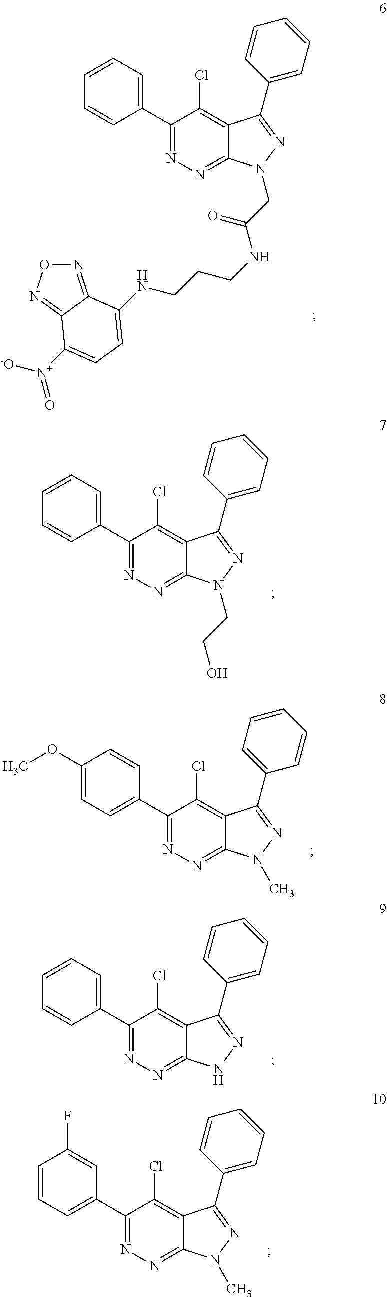

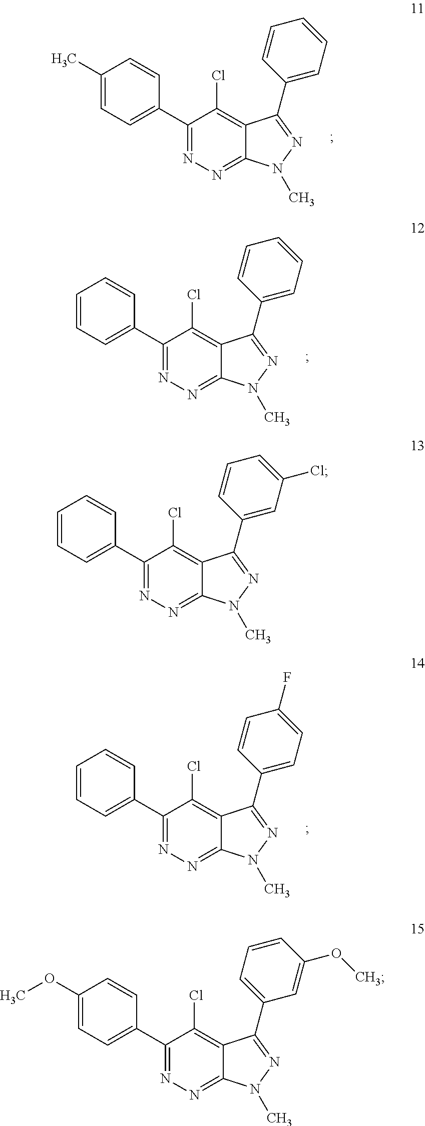

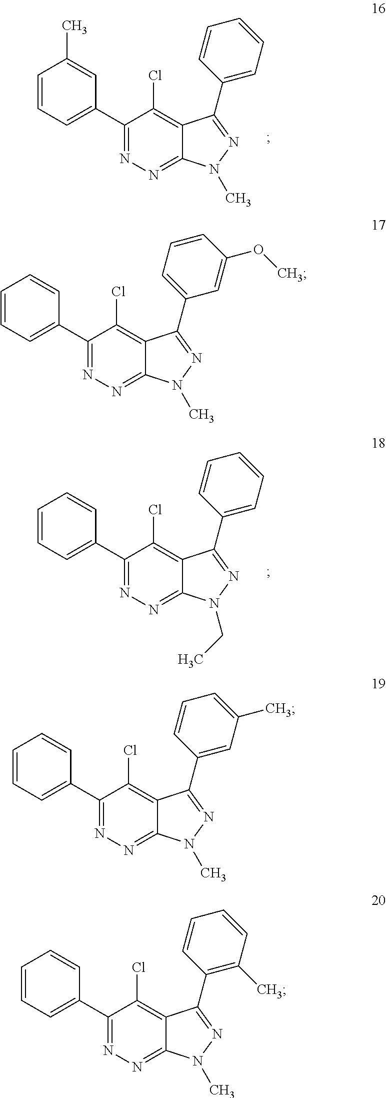

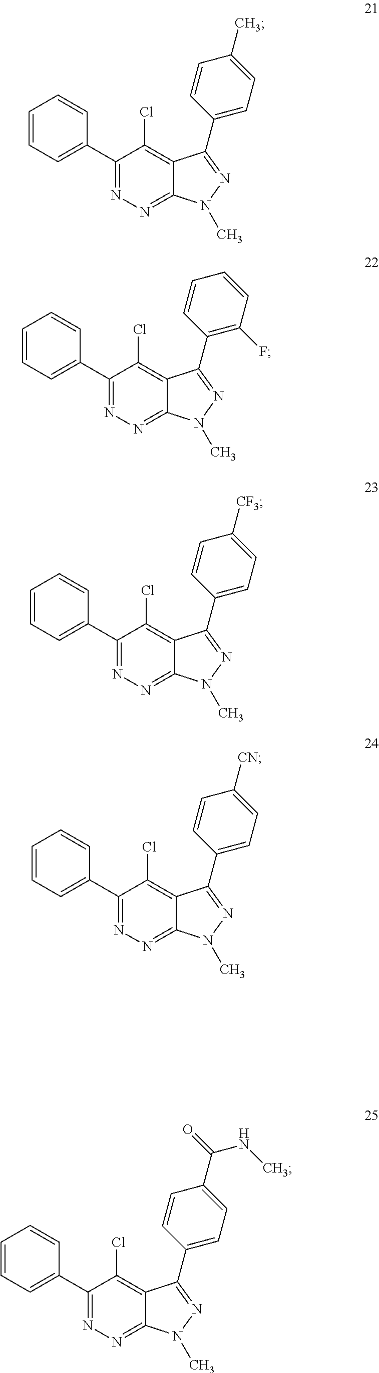

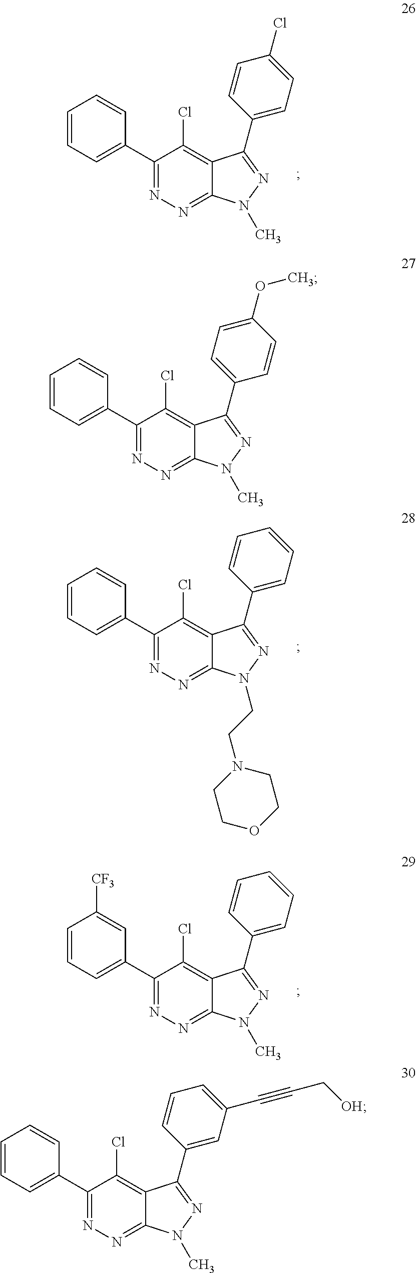

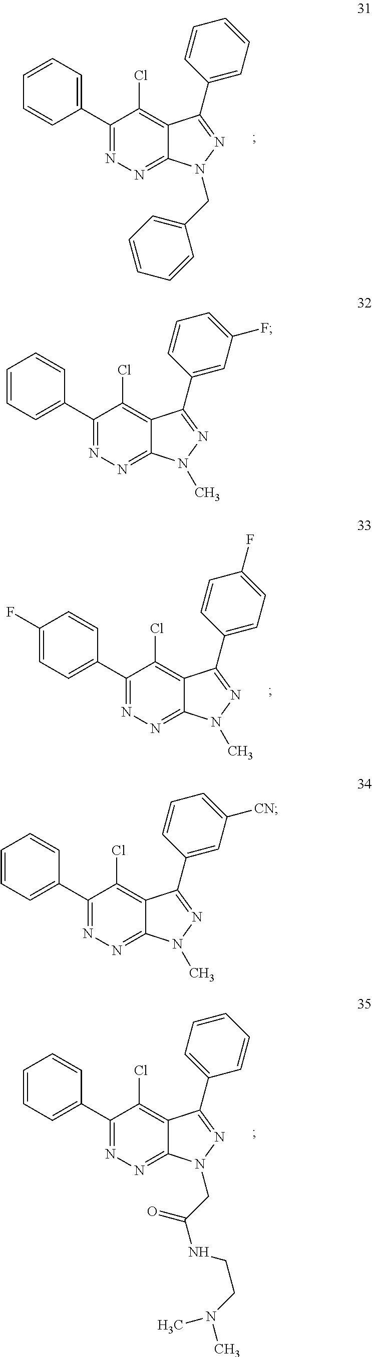

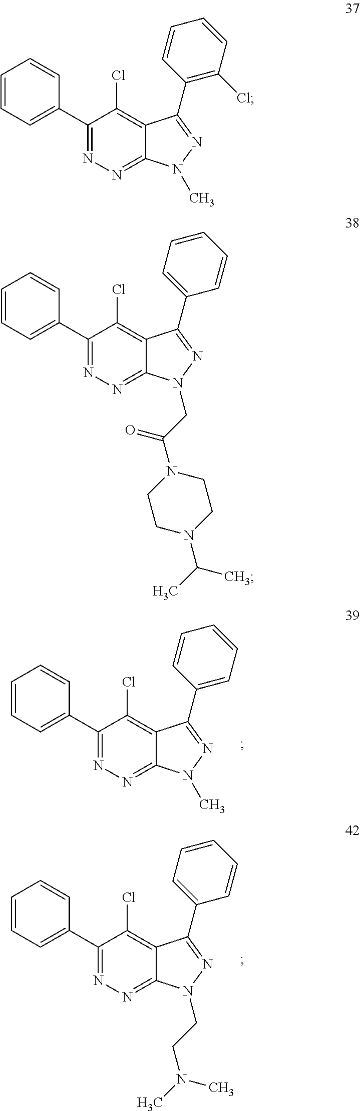

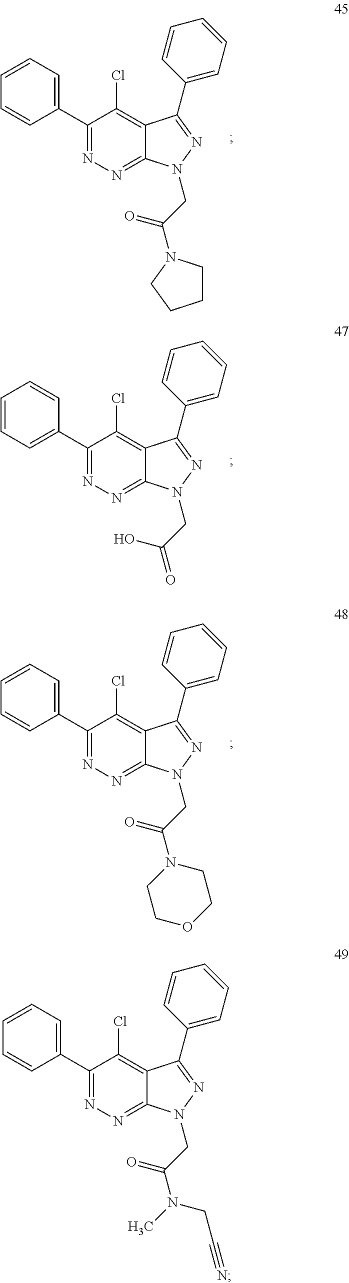

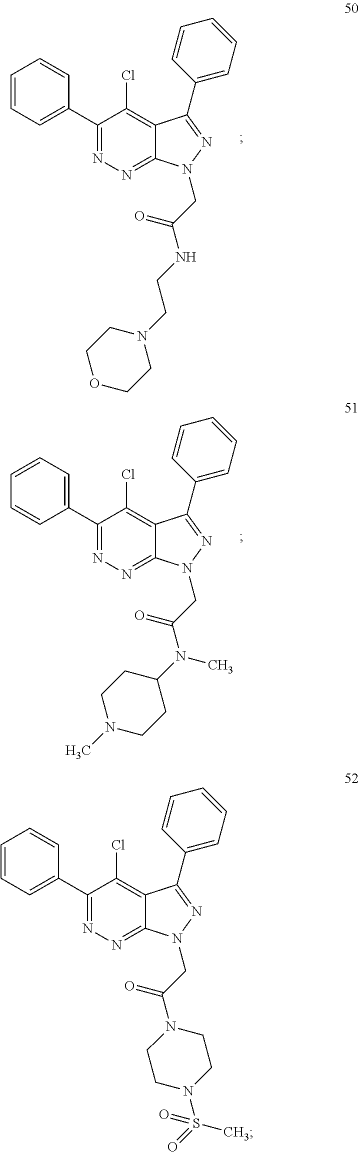 [SMILES: O=C(Cn1nc(-c2ccccc2)c2c(Cl)c(-c3ccccc3)nnc21)NCCN1CCOCC1 ; CN1CCC(N(C)C(=O)Cn2nc(-c3ccccc3)c3c(Cl)c(-c4ccccc4)nnc32)CC1 ; CS(=O)(=O)N1CCN(C(=O)Cn2nc(-c3ccccc3)c3c(Cl)c(-c4ccccc4)nnc32)CC1]

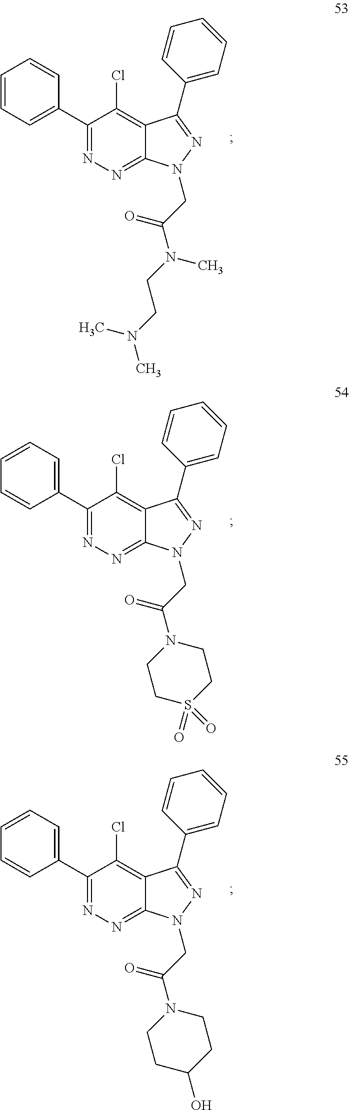

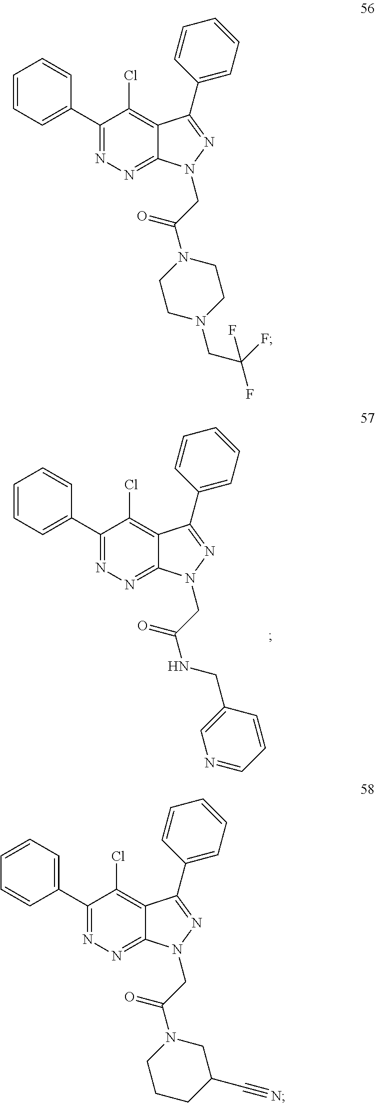

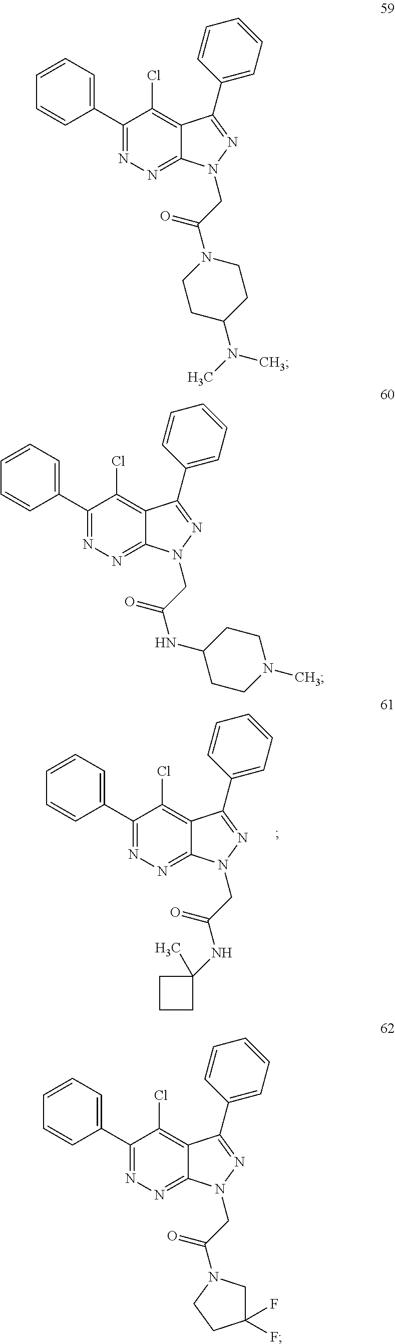

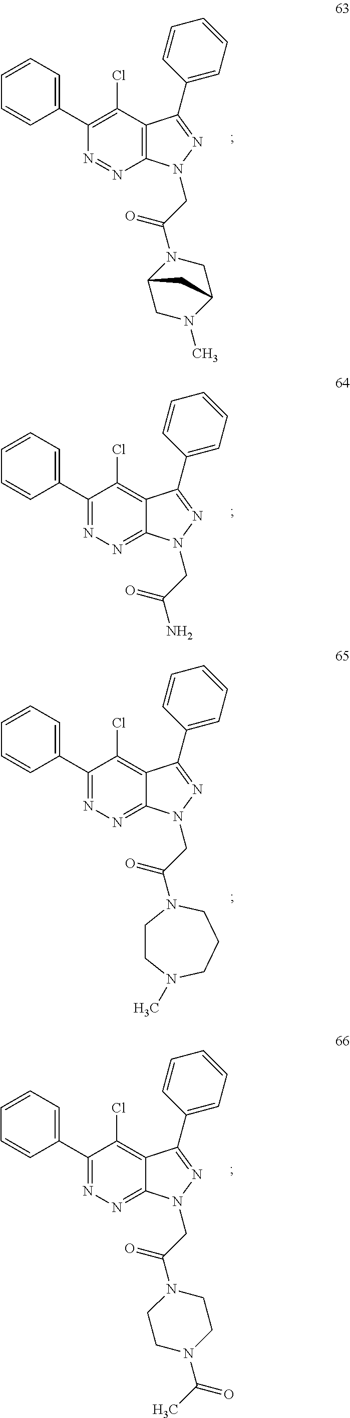

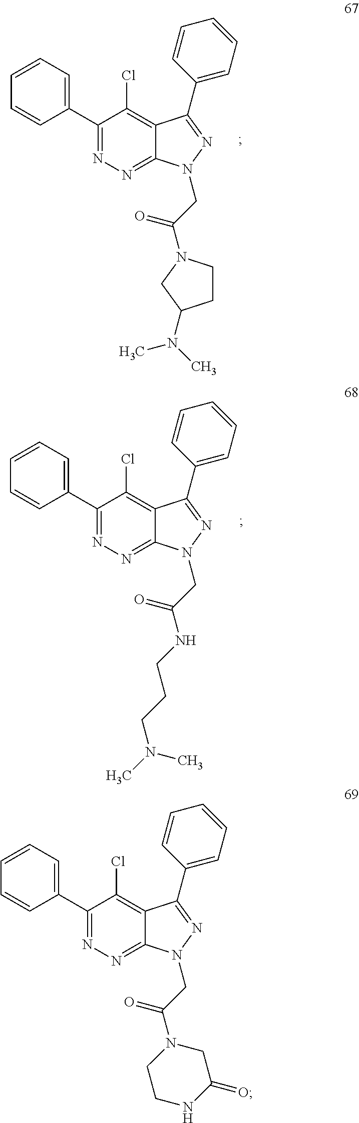

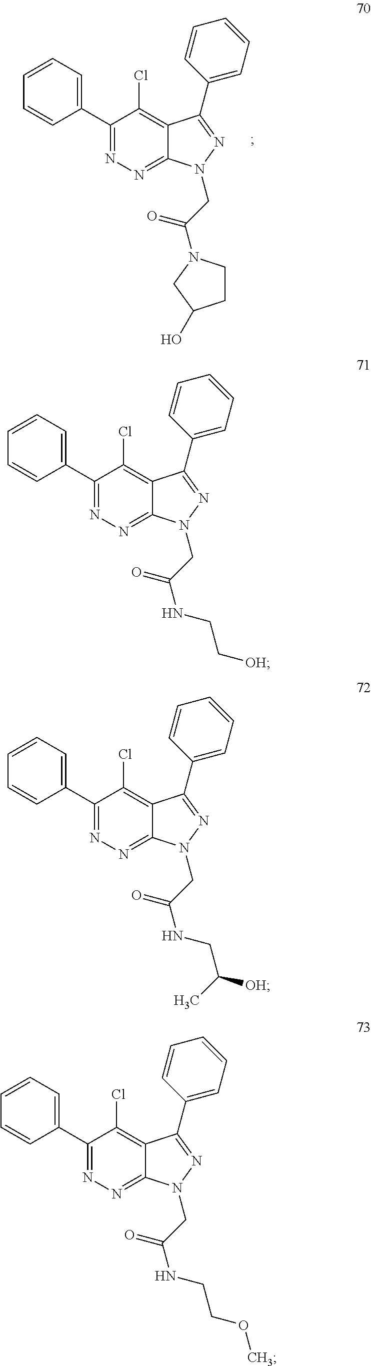

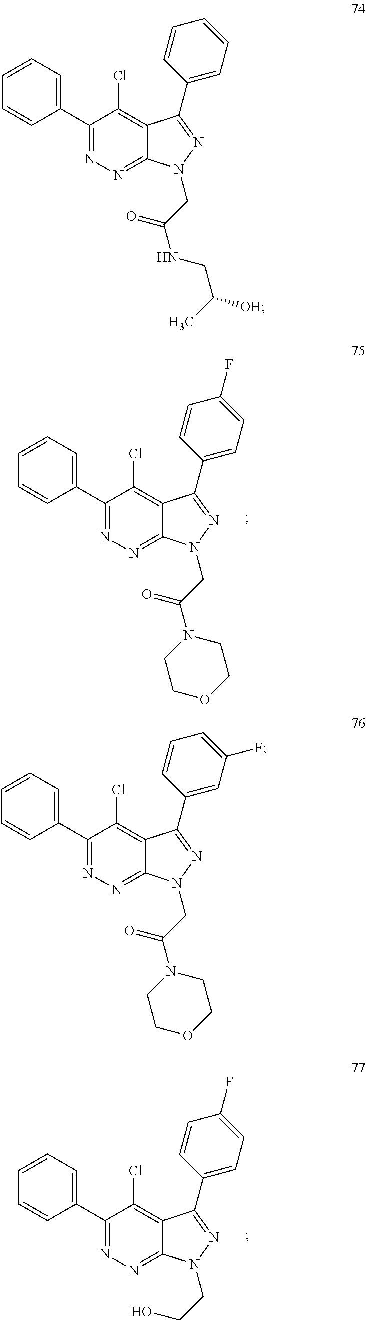

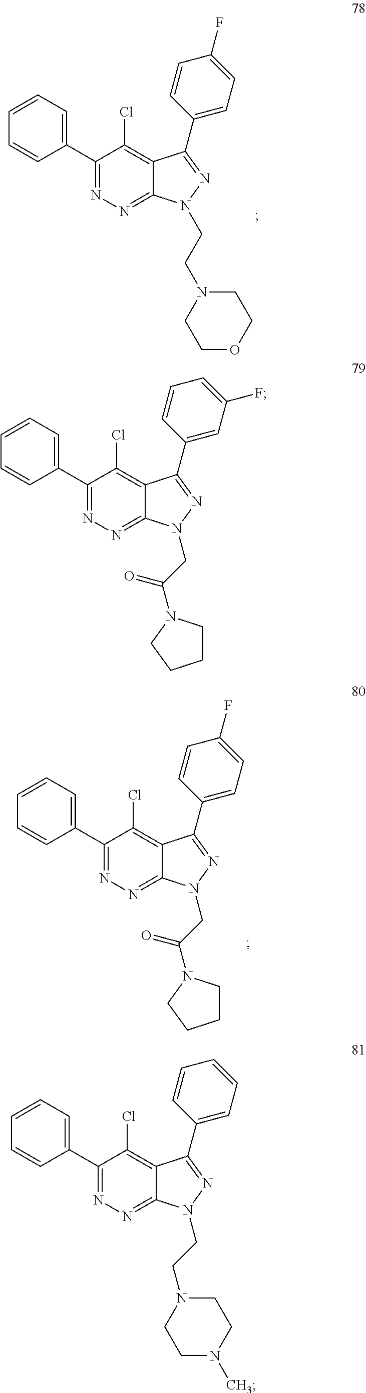

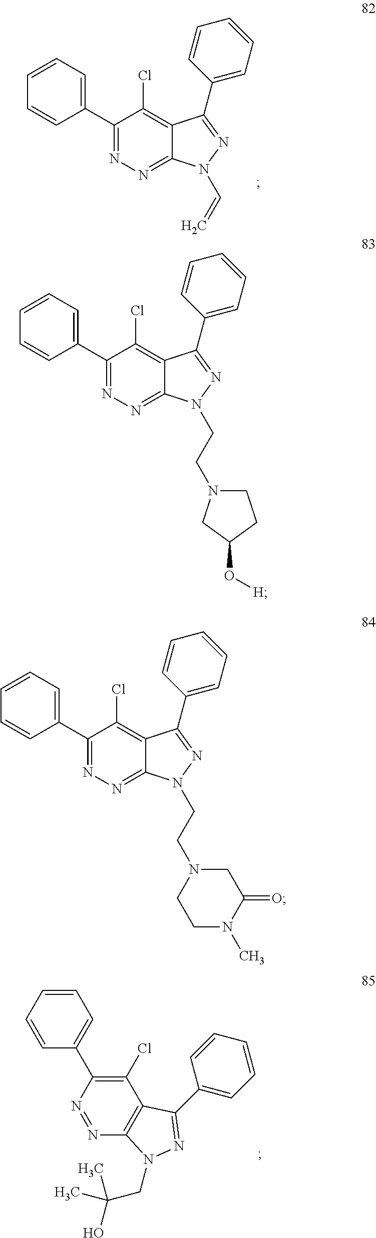

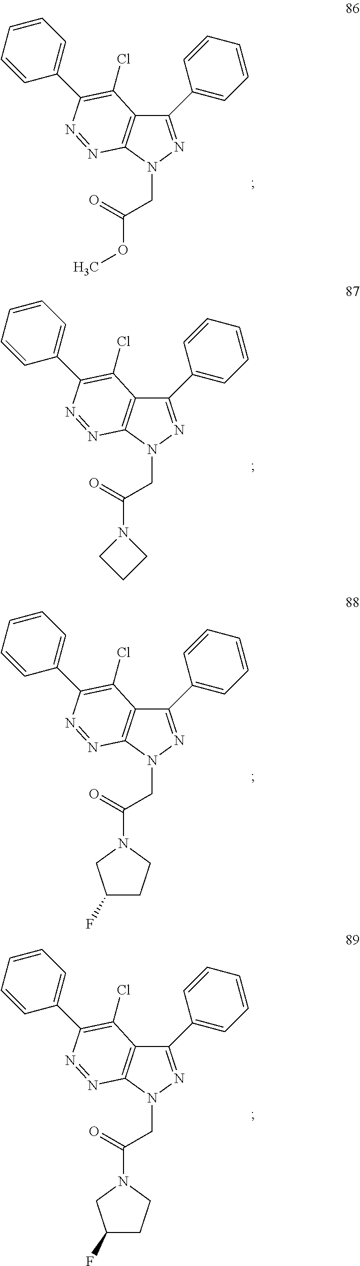 [SMILES: COC(=O)Cn1nc(-c2ccccc2)c2c(Cl)c(-c3ccccc3)nnc21 ; O=C(Cn1nc(-c2ccccc2)c2c(Cl)c(-c3ccccc3)nnc21)N1CCC1 ; O=C(Cn1nc(-c2ccccc2)c2c(Cl)c(-c3ccccc3)nnc21)N1CC[C@H](F)C1 ; O=C(Cn1nc(-c2ccccc2)c2c(Cl)c(-c3ccccc3)nnc21)N1CC[C@@H](F)C1]

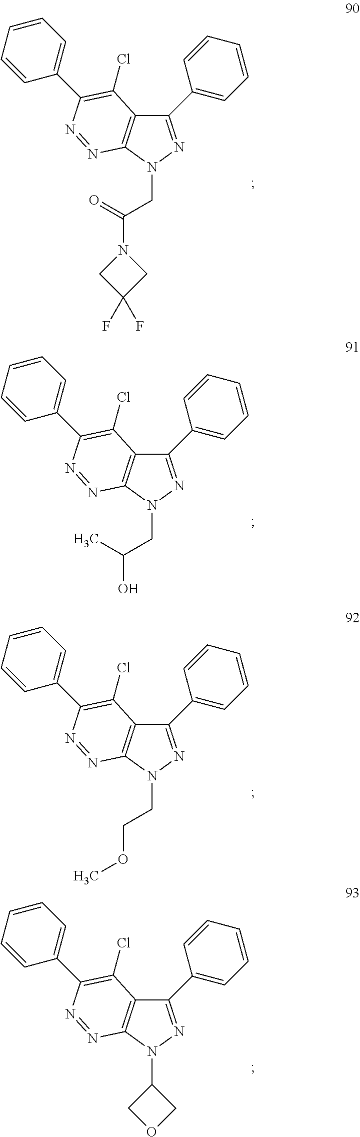 [SMILES: O=C(Cn1nc(-c2ccccc2)c2c(Cl)c(-c3ccccc3)nnc21)N1CC(F)(F)C1 ; CC(O)Cn1nc(-c2ccccc2)c2c(Cl)c(-c3ccccc3)nnc21 ; COCCn1nc(-c2ccccc2)c2c(Cl)c(-c3ccccc3)nnc21 ; Clc1c(-c2ccccc2)nnc2c1c(-c1ccccc1)nn2C1COC1]

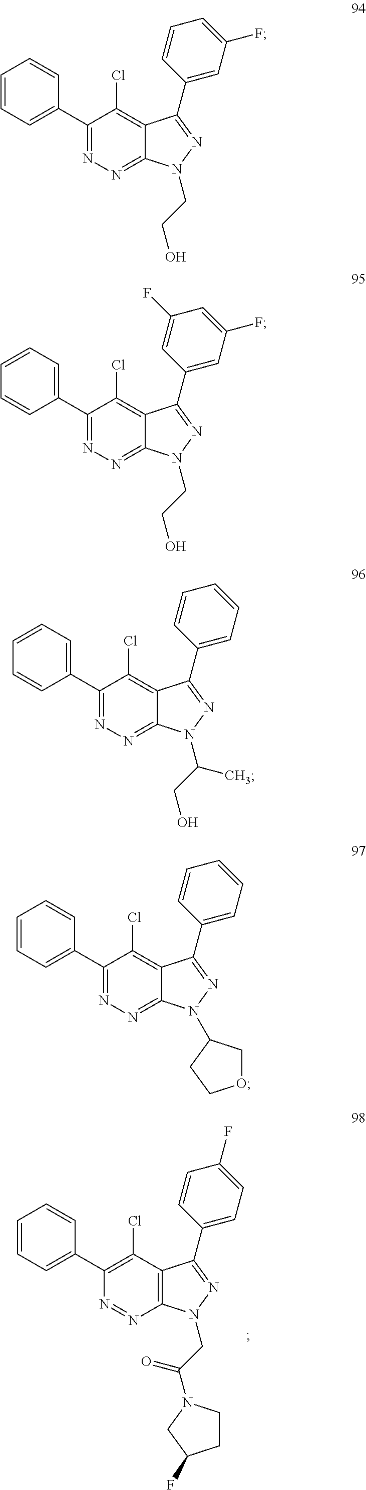

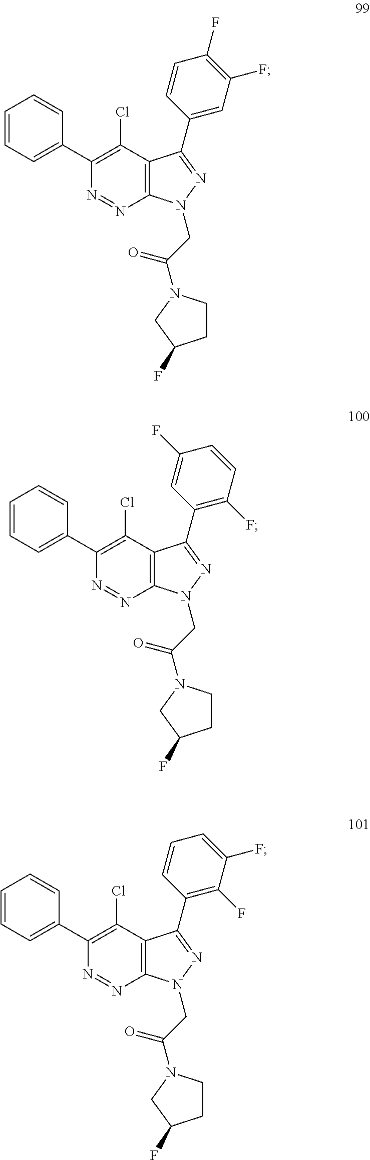 [SMILES: O=C(Cn1nc(-c2ccc(F)c(F)c2)c2c(Cl)c(-c3ccccc3)nnc21)N1CC[C@@H](F)C1 ; O=C(Cn1nc(-c2cc(F)ccc2F)c2c(Cl)c(-c3ccccc3)nnc21)N1CC[C@@H](F)C1 ; O=C(Cn1nc(-c2cccc(F)c2F)c2c(Cl)c(-c3ccccc3)nnc21)N1CC[C@@H](F)C1]

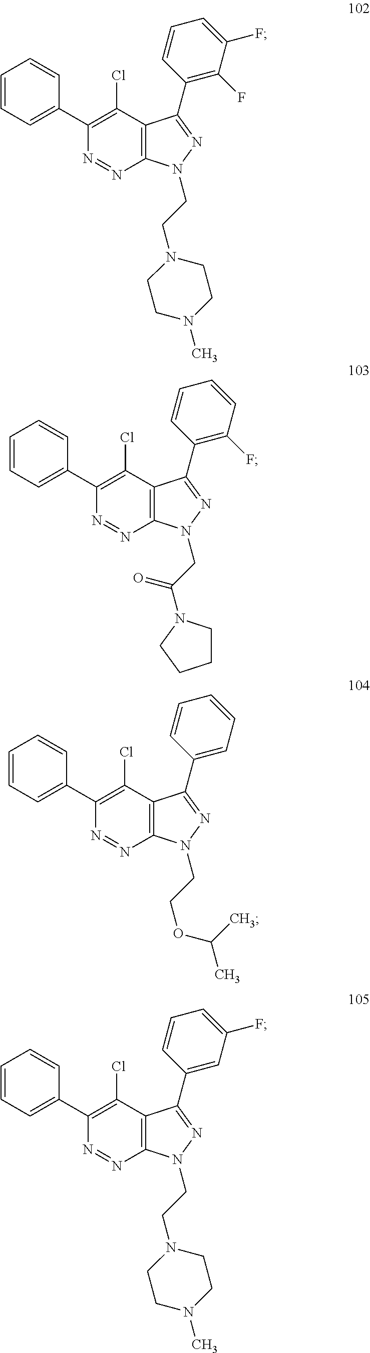

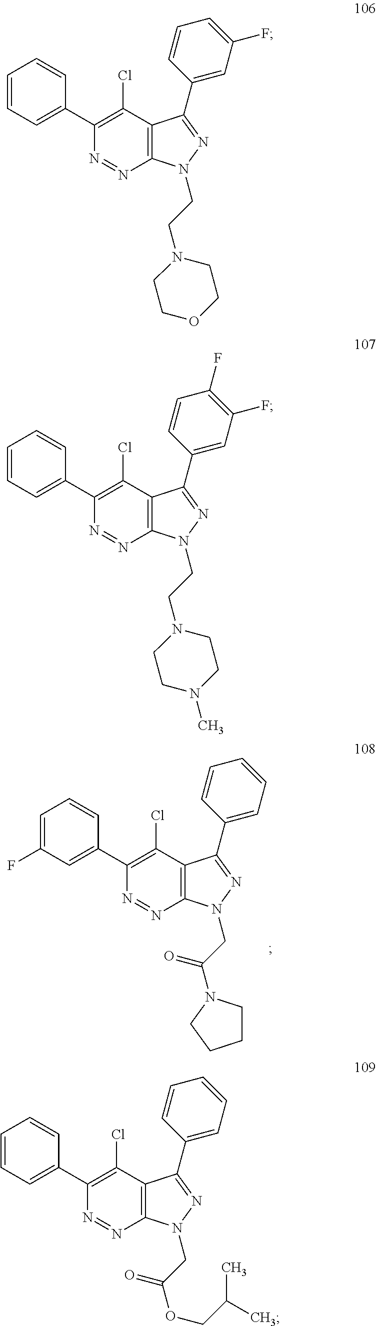

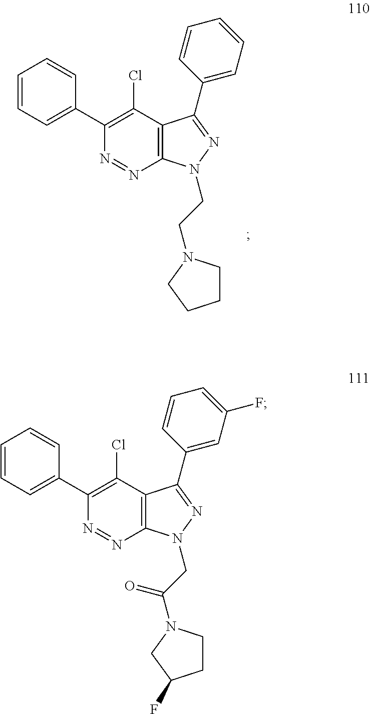 [SMILES: Clc1c(-c2ccccc2)nnc2c1c(-c1ccccc1)nn2CCN1CCCC1 ; O=C(Cn1nc(-c2cccc(F)c2)c2c(Cl)c(-c3ccccc3)nnc21)N1CC[C@@H](F)C1]

or a pharmaceutically acceptable salt thereof.

Pyrazolopyridazine Compounds of Formula III

The invention additionally provides methods for treating or preventing a proteopathy, comprising administering to a subject in need thereof an effective amount of a compound of Formula III:

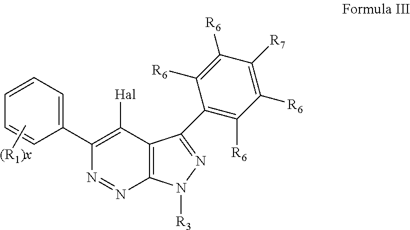

or a pharmaceutically acceptable salt thereof,
wherein Hal is —Cl, —F, —I, or —Br;
x is an integer ranging from 0 to 5;
each R₁ is independently —Cl, —F, —I, —Br, —C₁-C₃ alkyl, —O—C₁-C₃ alkyl, —CN, —CF₃, —C(O)NH(CH₃), or —C≡CCH₂OH;
R₃ is —H, —C₁-C₆ alkyl, —(C₁-C₆ alkylene)-OH, —(C₁-C₆ alkylene)-phenyl, —(C₁-C₆ alkylene)-O—(C₁-C₆ alkyl), —C₂-C₆ alkenyl, —(C₁-C₆ alkylene)-C(O)R₄, —(C₁-C₆ alkylene)-R₅,

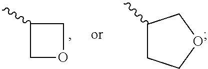

R₄ is —OH, —O—(C₁-C₆ alkyl), —NH₂, —NH(C₁-C₆ alkyl), —NH((C₁-C₆ alkylene)-OH), —NH((C₁-C₆ alkylene)N(C₁-C₆ alkyl)₂), —N(C₁-C₆ alkyl)((C₁-C₆ alkylene)-CN), —N(C₁-C₆ alkyl)((C₁-C₆ alkylene)N(C₁-C₆ alkyl)₂), —NH(C₁-C₆ alkylene)-O—(C₁-C₆ alkyl),

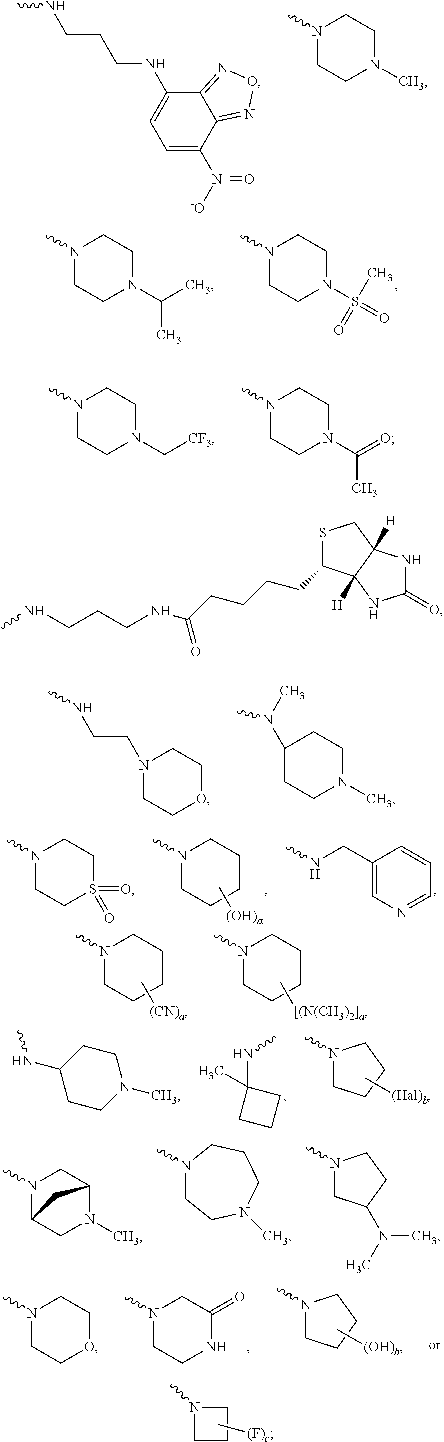

a is an integer ranging from 0 to 10;
b is an integer ranging from 0 to 8;
c is an integer ranging from 0 to 6;
R₅ is

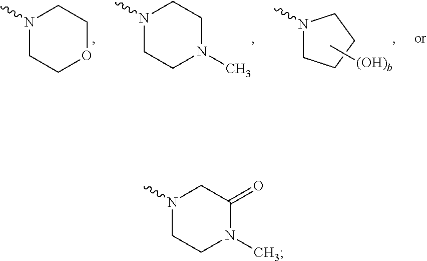

wherein each R₆ and R₇ is independently —H or —I, wherein at least one of R₆ and R₇ is —I,
and wherein when R₃ is —C₁-C₃ alkyl, R₇ is —H.

In certain embodiments, one R₆ in the ortho position relative to the pyrazolopyridazino ring system is iodo and the remaining R₆ and R₇ groups are hydrogen. In other embodiments, one R₆ in the para position relative to the pyrazolopyridazino ring system is iodo and the remaining R₆ and R₇ groups are hydrogen. In further embodiments, one R₆ in the ortho position relative to the pyrazolopyridazino ring system and one R₆ in the para position relative to the pyrazolopyridazino ring system are iodo and the remaining R₆ and R₇ groups are hydrogen. In further embodiments, the two R₆ groups in the ortho positions relative to the pyrazolopyridazino ring system and one R₆ in the para position relative to the pyrazolopyridazino ring system are iodo and the remaining R₆ and R₇ groups are hydrogen. In further embodiments, the two R₆ groups in the para positions relative to the pyrazolopyridazino ring system and one R₆ in the ortho position relative to the pyrazolopyridazino ring system are iodo and the remaining R₆ and R₇ are hydrogen. In certain embodiments, all R₆ groups are iodo and R₇ is hydrogen. In yet further embodiments, R₇ is is iodo and the R₆ groups are hydrogen.

In a particular embodiment, one R₆ in the para position relative to the pyrazolopyridazino ring system is iodo and R₃ is —CH₃.

In certain embodiments, Hal is —Cl. In yet another embodiment, x is 0. In another embodiment, x is 1. In a certain embodiments, x is 2.

In particular embodiments, x is 1 and R₁ is in the ortho position relative to the pyrazolopyridazino ring system. In certain embodiments, x is 1 and R₁ is in the para position relative pyrazolopyridazino ring system. In further embodiments, x is 1 and R₁ is in the meta position relative pyrazolopyridazino ring system.

In particular embodiments, x is 2 and R₁ is in the ortho and meta position relative pyrazolopyridazino ring system. In certain embodiments, x is 2 and R₁ is in the ortho and para position relative pyrazolopyridazino ring system. In further embodiments, x is 2 and R₁ is in the para and meta position relative pyrazolopyridazino ring system.

In yet other embodiments, R₁ is —Cl. In certain embodiments, R₁ is —F. In certain embodiments, R₁ is —I. In further embodiments, R₁ is —OCH₃. In other embodiments, R₁ is —CH₃. In yet other embodiments, R₁ is —C(O)N(H)CH₃. In certain embodiments, R₁ is —CF₃. In further embodiments, R₁ is —CN. In additional embodiments, R₁ is —C≡CCH₂OH.

In yet other embodiments, x is 1 or 2, and R₁ is —Cl, —F, —Br, —I, —OCH₃, —CH₃, —C(O)N(H)CH₃, —CF₃, —CN or —C≡CCH₂OH.

In yet other embodiments, Hal is —Cl, x is 1 or 2, and R₁ is —Cl, —F, —Br, —I, —OCH₃, —CH₃, —C(O)N(H)CH₃, —CF₃, —CN or —C≡CCH₂OH.

In particular embodiments, R₃ is —H. In certain embodiments, R₃ is —CH₃. In further embodiments, R₃ is —CH₂CH₃. In still further embodiments, R₃ is —CHCH₂. In other embodiments, R₃ is —CH₂CH₂OH. In particular embodiments, R₃ is —(CH₂)₂C₆H₅. In other embodiments, R₃ is —CH₂C(O)OH. In yet other embodiments, R₃ is —CH₂C(O)N(H)CH₃. In certain embodiments, R₃ is —CH₂C(O)N(H)((CH₂)₂N(CH₃)₂). In yet other embodiments, R₃ is —CH₂C(O)N(H)((CH₂)₃N(CH₃)₂). In other embodiments, R₃ is—CH₂C(O)N(CH₃)CH₂CN. In particular embodiments, R₃ is —CH₂C(O)NH₂. In certain embodiments, R₃ is —CH₂C(O)N(H)((CH₂)₂OH). In other embodiments, R₃ is —CH₂C(O)N(H)((CH₂)₂OCH₃). In still further embodiments, R₃ is —CH₂C(CH₃)₂OH. In yet other embodiments, R₃ is —CH₂C(O)OCH₃. In further embodiments, R₃ is —CH₂CH(OH)CH₃. In still further embodiments, R₃ is —CH₂CH₂OH. In particular embodiments, R₃ is —CH(CH₃)CH₂OH.

In further embodiments, R₃ is —CH₂C(O)R₄ and R₄ is

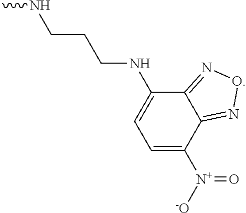

In other embodiments, R₃ is —CH₂C(O)R₄ and R₄ is

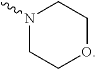

In particular embodiments, R₃ is —CH₂C(O)R₄ and R₄ is

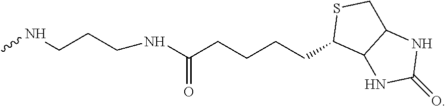

In yet other embodiments, R₃ is —CH₂C(O)R₄ and R₄ is

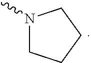

In certain embodiments, R₃ is —CH₂C(O)R₄ and R₄ is

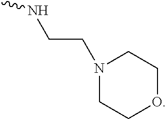

In other embodiments, R₃ is —CH₂C(O)R₄ and R₄ is

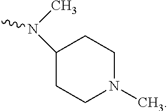

In particular embodiments, R₃ is —CH₂C(O)R₄ and R₄ is

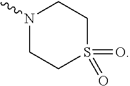

In yet other embodiments, R₃ is —CH₂C(O)R₄ and R₄ is

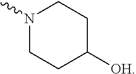

In certain embodiments, R₃ is —CH₂C(O)R₄ and R₄ is

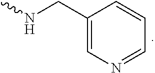

In other embodiments, R₃ is —CH₂C(O)R₄ and R₄ is

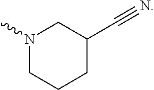

In particular embodiments, R₃ is —CH₂C(O)R₄ and R₄ is

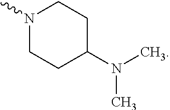

In yet other embodiments, R₃ is —CH₂C(O)R₄ and R₄ is

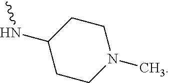

In certain embodiments, R₃ is —CH₂C(O)R₄ and R₄ is

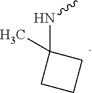

In other embodiments, R₃ is —CH₂C(O)R₄ and R₄ is

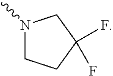

In particular embodiments, R₃ is —CH₂C(O)R₄ and R₄ is

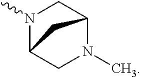

In yet other embodiments, R₃ is —CH₂C(O)R₄ and R₄ is

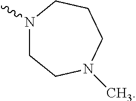

In certain embodiments, R₃ is —CH₂C(O)R₄ and R₄ is

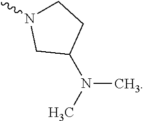

In other embodiments, R₃ is —CH₂C(O)R₄ and R₄ is

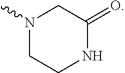

In particular embodiments, R₃ is —CH₂C(O)R₄ and R₄ is

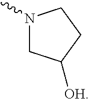

In yet other embodiments, R₃ is —CH₂C(O)R₄ and R₄ is

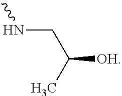

In certain embodiments, R₃ is —CH₂C(O)R₄ and R₄ is

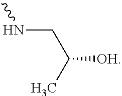

In other embodiments, R₃ is —CH₂C(O)R₄ and R₄ is

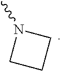

In particular embodiments, R₃ is —CH₂C(O)R₄ and R₄ is

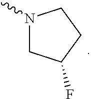

In yet other embodiments, R₃ is —CH₂C(O)R₄ and R₄ is

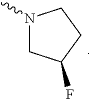

In certain embodiments, R₃ is —CH₂C(O)R₄ and R₄ is

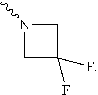

In other embodiments, R₃ is —CH₂C(O)R₄ and R₄ is

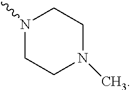

In further embodiments of the invention, R₃ is —CH₂C(O)R₄ and R₄ is

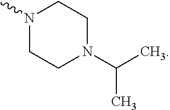

In certain embodiments of the invention, R₃ is —CH₂C(O)R₄ and R₄ is

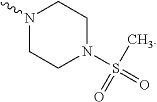

In other embodiments, R₃ is —CH₂C(O)R₄ and R₄ is

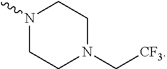

In further embodiments, R₃ is —CH₂C(O)R₄ and R₄ is

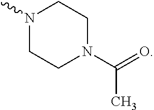

In further embodiments, R₃ is —CH₂C(O)R₄ and R₄ is

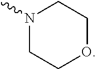

In particular embodiments, R₃ is —(CH ₂)₂R₅ and R₅ is

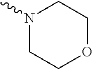

In yet other embodiments, R₃ is —(CH₂)₂R₅ and R₅ is

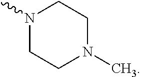

In certain embodiments, R₃ is —(CH₂)₂R₅ and R₅ is

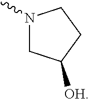

In other embodiments, invention, R₃ is —(CH₂)₂R₅ and R₅ is

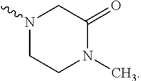

In some embodiments, a is an integer ranging from 0 to 5. In some embodiments, b is an integer ranging from 0 to 4. In some embodiments, c is an integer ranging from 0 to 6.

In certain embodiments, the compound of Formula III is Compound 3, which has the structure:

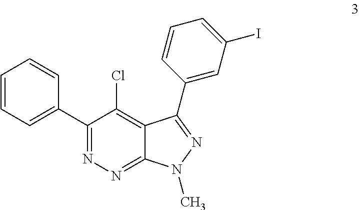 [SMILES: Cn1nc(-c2cccc(I)c2)c2c(Cl)c(-c3ccccc3)nnc21]

or a pharmaceutically acceptable salt thereof.

Pyrazolopyridazine Compounds of Formula IV

The invention additionally provides methods for treating or preventing a proteopathy, comprising administering to a subject in need thereof an effective amount of a compound of Formula IV:

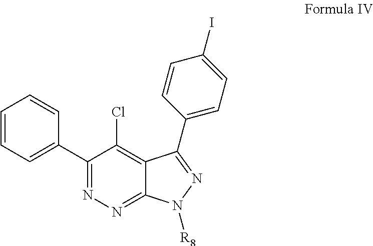

or a pharmaceutically acceptable salt thereof,
wherein R₈ is —C₁-C₃ alkyl.

In certain embodiments of the invention, R₈ is —CH₃, in yet further embodiments of the invention, R₈ is —CH₂CH₃. In other embodiments of the invention, R₈ is —CH₂CH₂CH₃. In other embodiments of the invention, R₈ is —CH(CH₃)₂.

In certain embodiments, the compound of Formula IV is Compound 43, which has the structure:

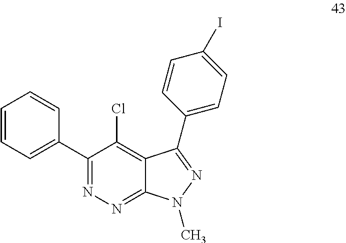 [SMILES: Cn1nc(-c2ccc(I)cc2)c2c(Cl)c(-c3ccccc3)nnc21]

or a pharmaceutically acceptable salt thereof.

Pyrazolopyridazine Compounds of Formula V

The invention further provides methods for treating or preventing a proteopathy, comprising administering to a subject in need thereof an effective amount of a compound of Formula V:

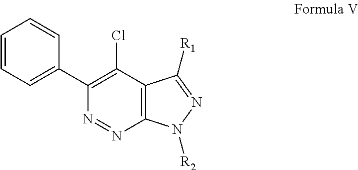

or a pharmaceutically acceptable salt thereof,
wherein R₁ is:

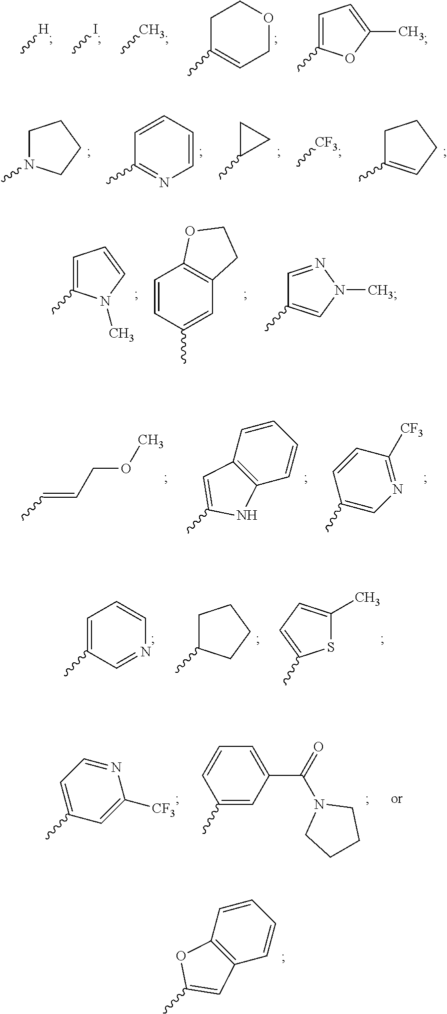

R₂ is:

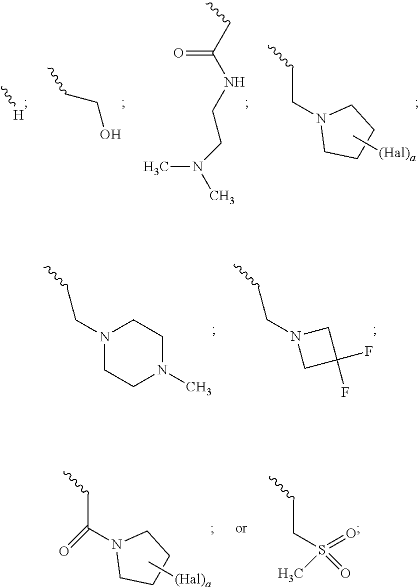

Hal is —Cl, —F, —I, or —Br; and
a is 0, 1, or 2.

In particular embodiments, R₁ is —I. In other embodiments, R₁ is —H. In yet other embodiments, R₁ is —CH₃. In certain embodiments, R₁ is —CF₃.

In yet other embodiments, R₁ is

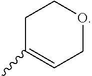

In certain embodiments, R₁ is

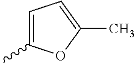

In still further embodiments, R₁ is

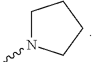

In particular embodiments, R₁ is

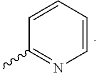

In other embodiments, R₁ is

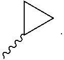

In yet other embodiments, R₁ is

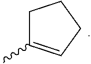

In certain embodiments, R₁ is

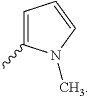

In particular embodiments, R₁ is

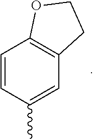

In certain embodiments, R₁ is

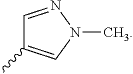

In still further embodiments, R₁ is

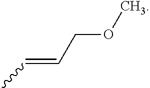

In other embodiments, R₁ is

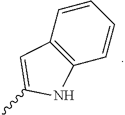

In yet other embodiments, R₁ is

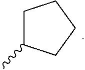

In certain embodiments, R₁ is

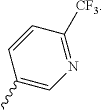

In still further embodiments, R₁ is

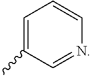

In other embodiments, R₁ is

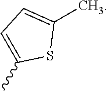

In particular embodiments, R₁ is

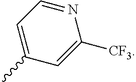

In further embodiments, R₁ is

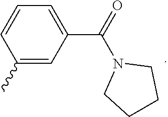

In still further embodiments, R₁ is

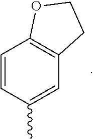

In certain embodiments, R₂ is —H. In yet other embodiments, R₂ is

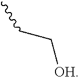

In particular embodiments, R₂ is

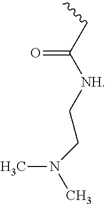

In yet other embodiments, R₂ is

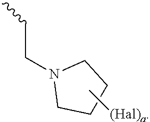

In further embodiments, R₂ is

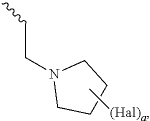

a=1, and Hal is —F.

In certain embodiments, R₂ is

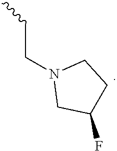

In still further embodiments, R₂ is

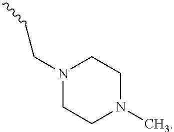

In particular embodiments, R₂ is

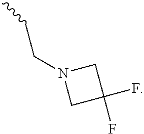

In other embodiments, R₂ is

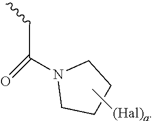

In yet other embodiments, R₂ is

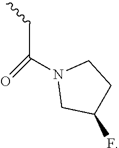

In certain embodiments, R₂ is

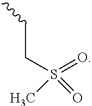

In further embodiments, when a is 2, each Hal is the same or different.

In certain embodiments the compound of Formula V has the structure:

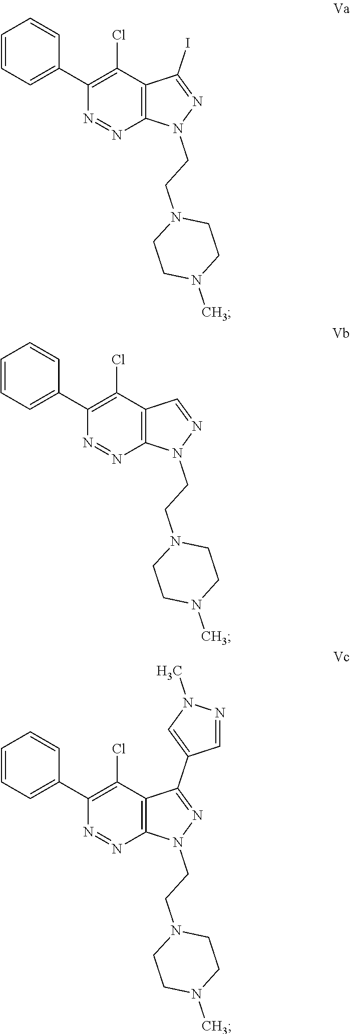

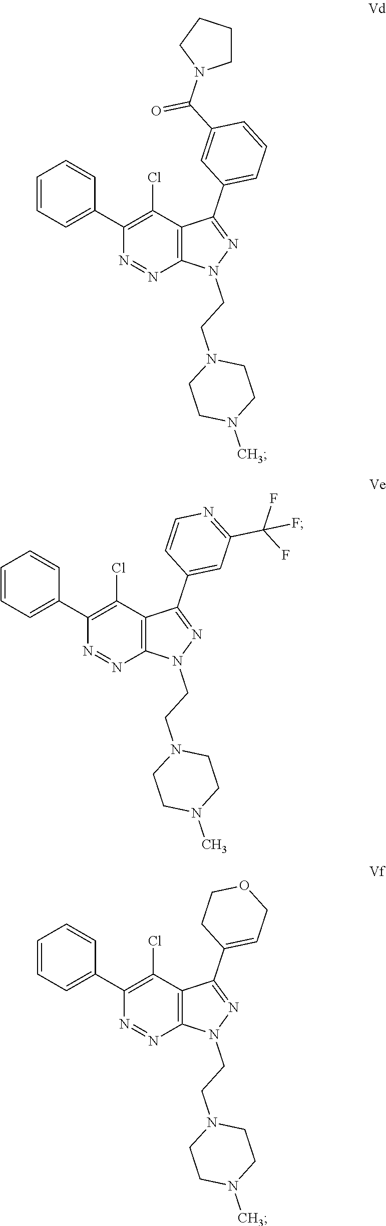 [SMILES: CN1CCN(CCn2nc(-c3cccc(C(=O)N4CCCC4)c3)c3c(Cl)c(-c4ccccc4)nnc32)CC1 ; CN1CCN(CCn2nc(-c3ccnc(C(F)(F)F)c3)c3c(Cl)c(-c4ccccc4)nnc32)CC1 ; CN1CCN(CCn2nc(C3=CCOCC3)c3c(Cl)c(-c4ccccc4)nnc32)CC1]

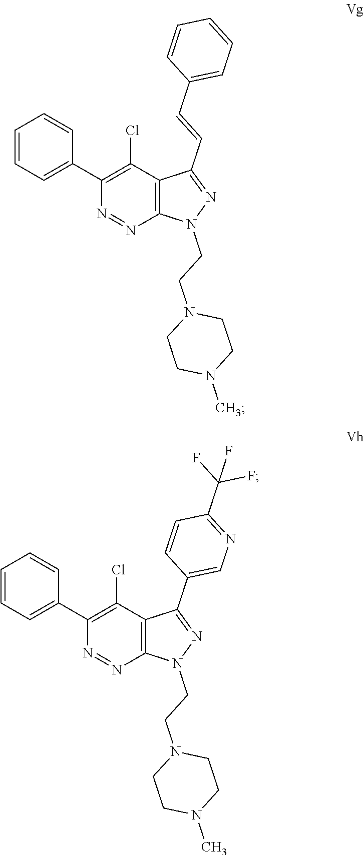

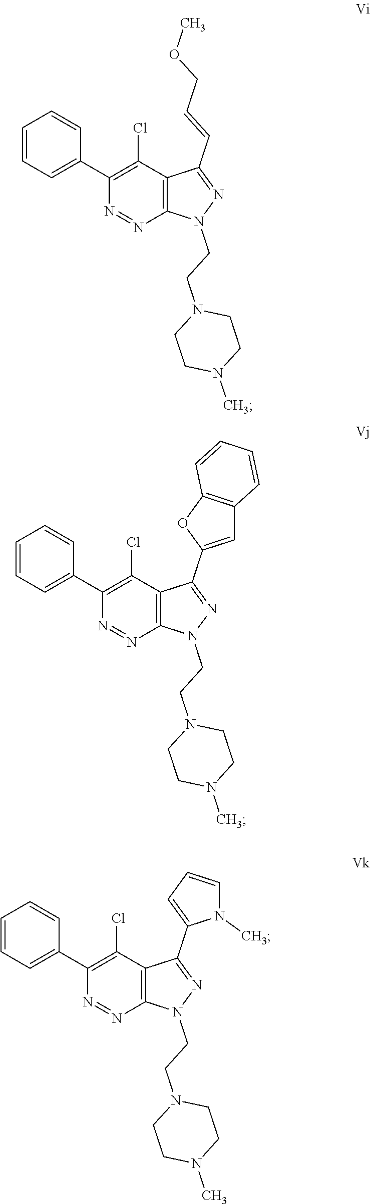

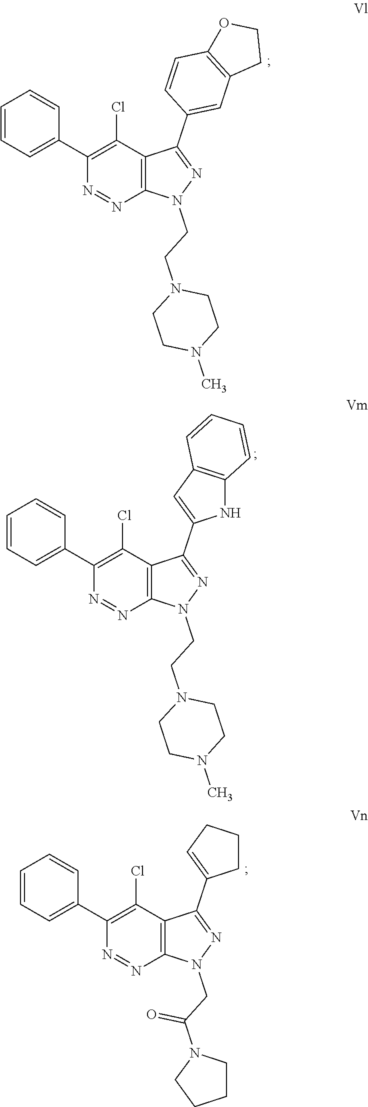

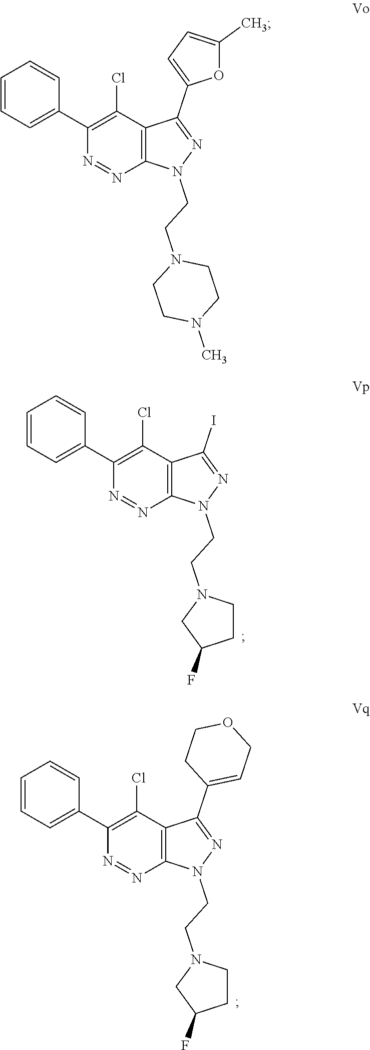 [SMILES: Cc1ccc(-c2nn(CCN3CCN(C)CC3)c3nnc(-c4ccccc4)c(Cl)c23)o1 ; F[C@@H]1CCN(CCn2nc(I)c3c(Cl)c(-c4ccccc4)nnc32)C1 ; F[C@@H]1CCN(CCn2nc(C3=CCOCC3)c3c(Cl)c(-c4ccccc4)nnc32)C1]

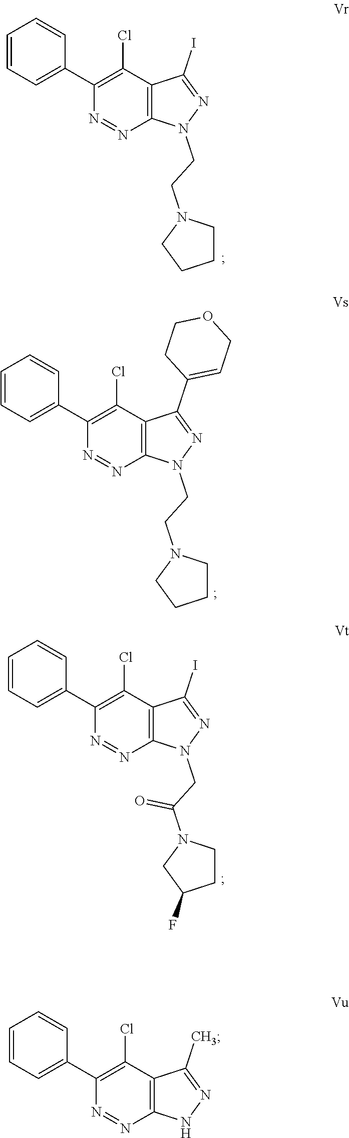 [SMILES: Clc1c(-c2ccccc2)nnc2c1c(I)nn2CCN1CCCC1 ; Clc1c(-c2ccccc2)nnc2c1c(C1=CCOCC1)nn2CCN1CCCC1 ; O=C(Cn1nc(I)c2c(Cl)c(-c3ccccc3)nnc21)N1CC[C@@H](F)C1 ; Cc1n[nH]c2nnc(-c3ccccc3)c(Cl)c12]

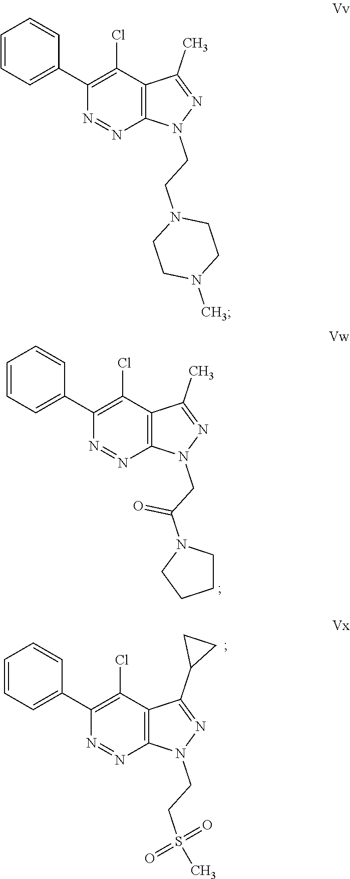

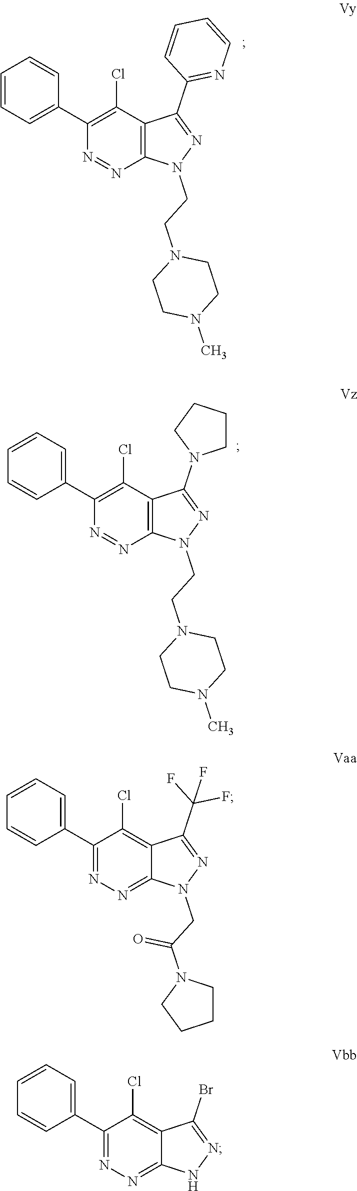

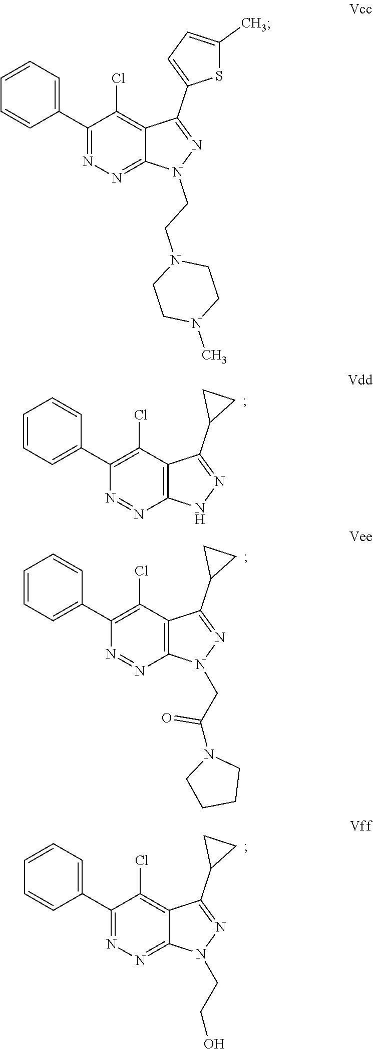

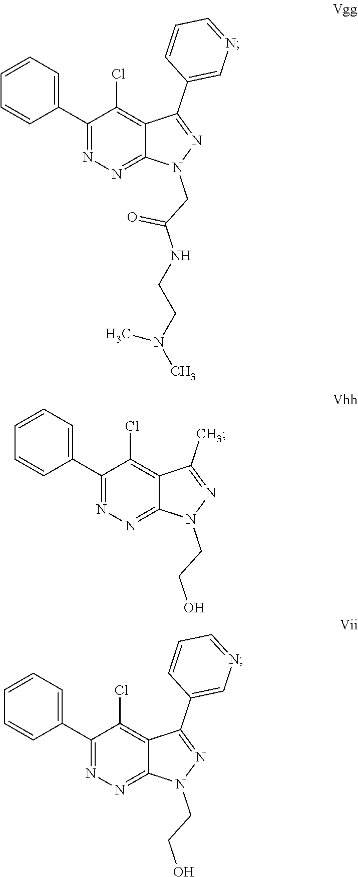

or a pharmaceutically acceptable salt thereof.

Pyrazolopyridazine Compounds of Formula VI

The invention also provides methods for treating or preventing a proteopathy, comprising administering to a subject in need thereof an effective amount of a compound of Formula VI:

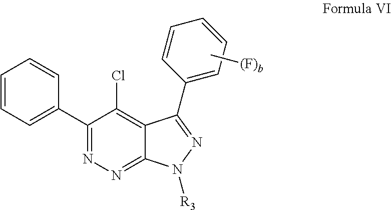

or a pharmaceutically acceptable salt thereof,
wherein R₃ is:

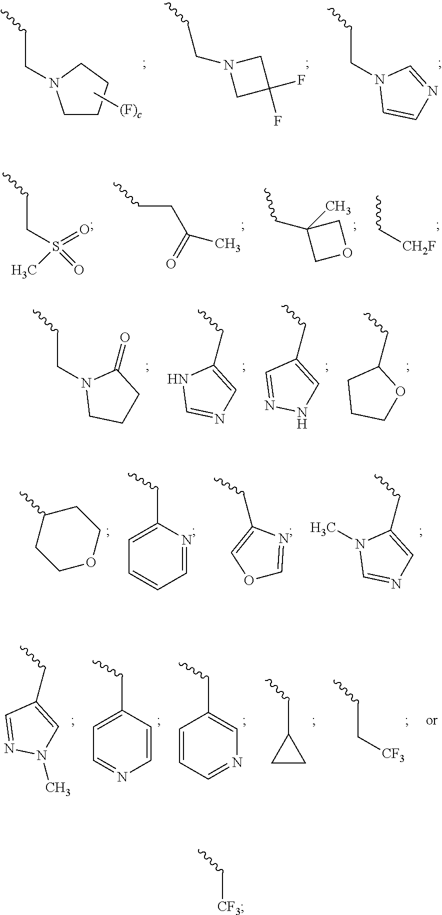

b is 0 or 1; and
c is 1 or 2.

In particular embodiments, b is 0. In other embodiments b is 1 and the —F is in the meta position relative to the pyrazolopyridazino ring system. In yet other embodiments b is 1 and the —F is in the para position relative to the pyrazolopyridazino ring system.

In particular embodiments R₃ is —CF₃. In certain embodiments R₃ is

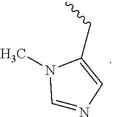

In other embodiments R₃ is

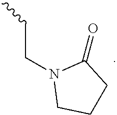

In yet other embodiments R₃ is

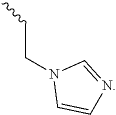

In further embodiments R₃ is

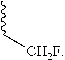

In still further embodiments R₃ is

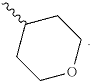

In particular embodiments R₃ is

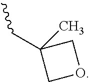

In other embodiments R₃ is

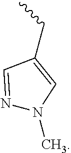

In yet other embodiments R₃ is

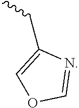

In certain embodiments R₃ is

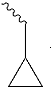

In further embodiments R₃ is

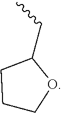

In further embodiments R₃ is

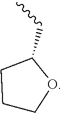

In certain embodiments R₃ is

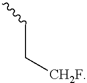

In other embodiments R₃ is

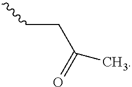

In yet other embodiments R₃ is

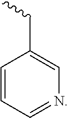

In further embodiments R₃ is

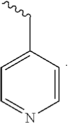

In still further embodiments R₃ is

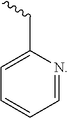

In particular embodiments R₃ is

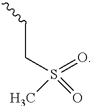

In certain embodiments R₃ is

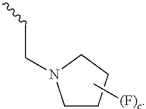

In further embodiments R₃ is

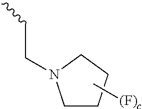

and c=1. In still further embodiments R₃ is

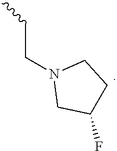

In particular embodiments R₃ is

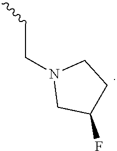

In other embodiments R₃ is

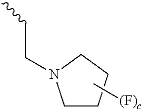

and c=2. In yet other embodiments R₃ is

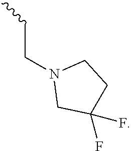

In certain embodiments R₃ is

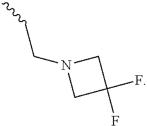

In other embodiments R₃ is

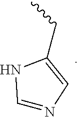

In yet other embodiments R₃ is

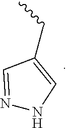

In certain embodiments the compound of Formula VI has the structure:

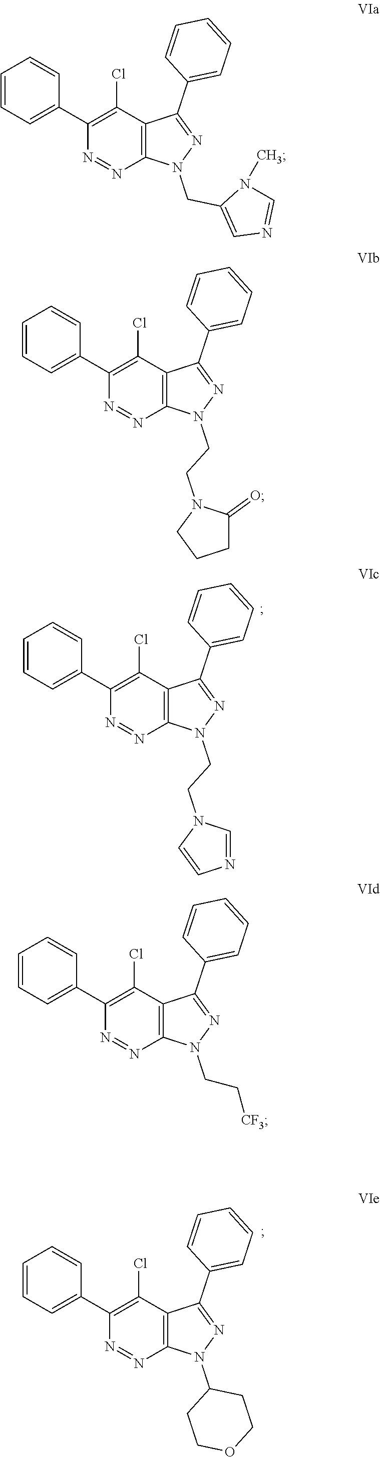

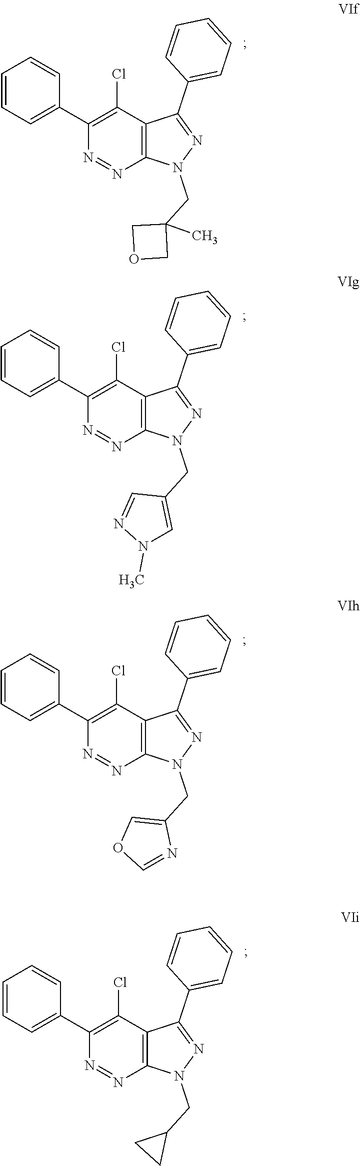

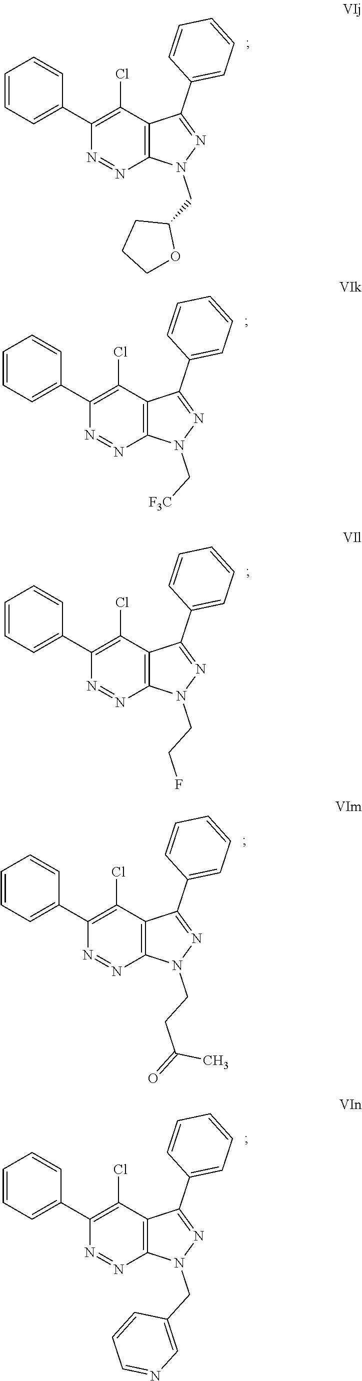

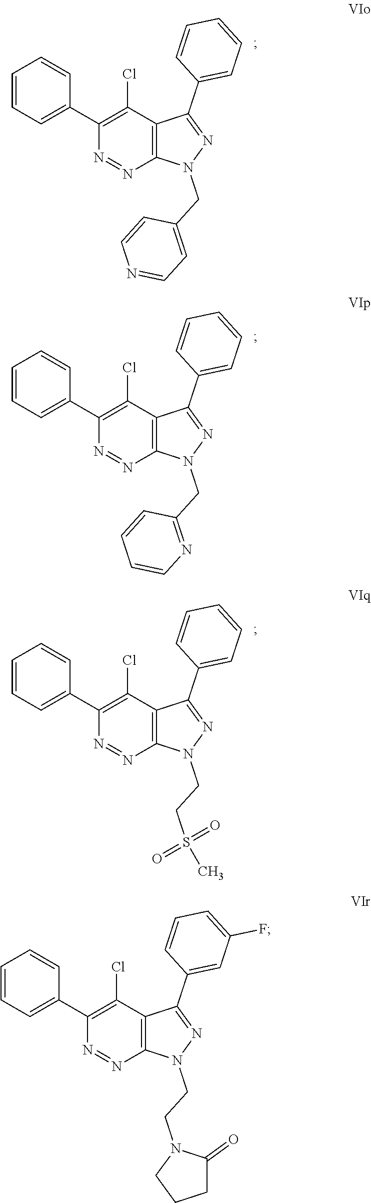

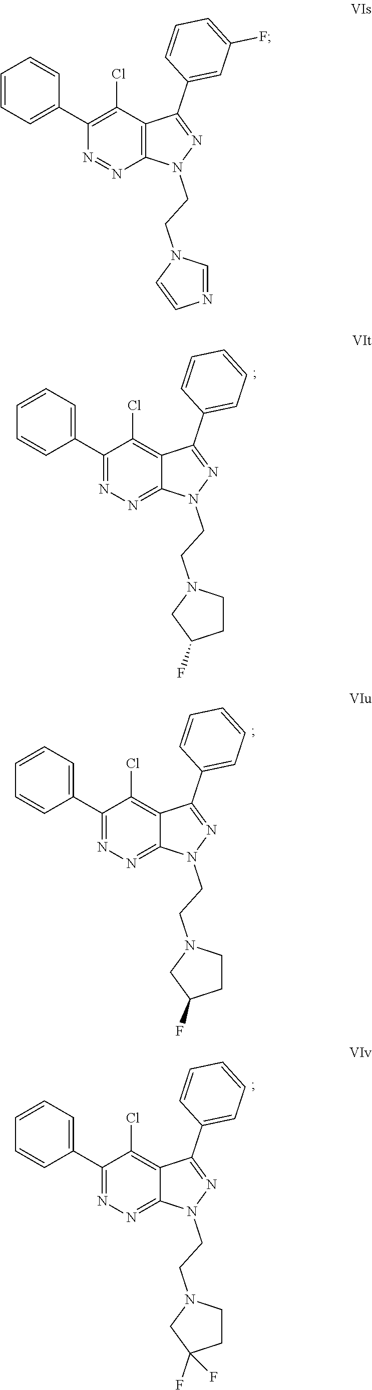

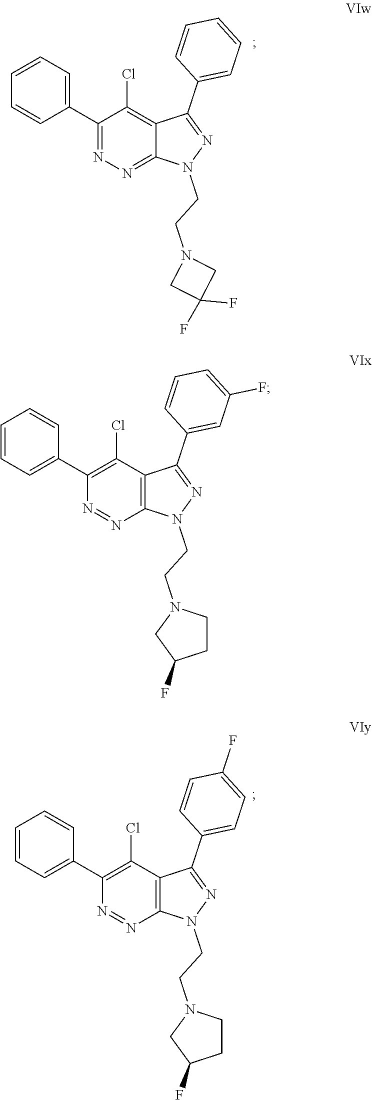 [SMILES: FC1(F)CN(CCn2nc(-c3ccccc3)c3c(Cl)c(-c4ccccc4)nnc32)C1 ; Fc1cccc(-c2nn(CCN3CC[C@@H](F)C3)c3nnc(-c4ccccc4)c(Cl)c23)c1 ; Fc1ccc(-c2nn(CCN3CC[C@@H](F)C3)c3nnc(-c4ccccc4)c(Cl)c23)cc1]

or a pharmaceutically acceptable salt thereof.

Pyrazolopyridazine Compounds of Formula VII

The invention additionally provides methods for treating or preventing a proteopathy, comprising administering to a subject in need thereof an effective amount of a compound of Formula VII:

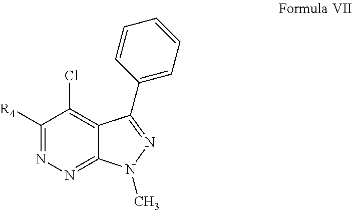

or a pharmaceutically acceptable salt thereof,
wherein R₄ is

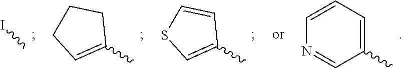

In certain embodiments R₄ is

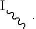

In particular embodiments R₄ is

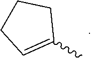

In other embodiments R₄ is

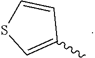

In yet other embodiments R₄ is

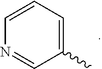

In certain embodiments the compound of Formula VII has the structure:

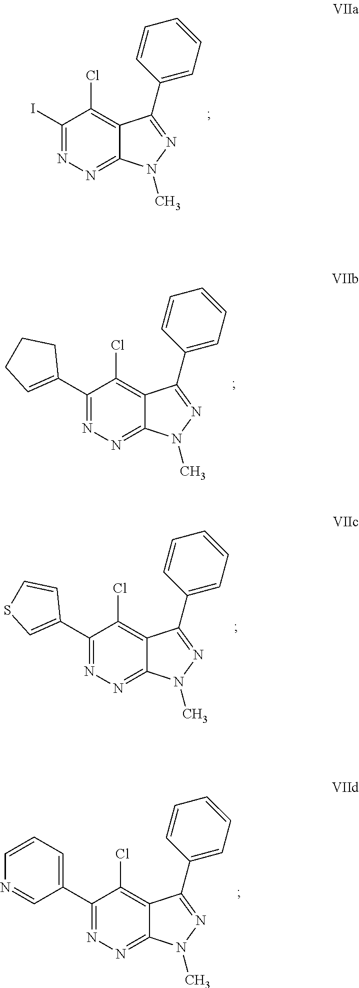 [SMILES: Cn1nc(-c2ccccc2)c2c(Cl)c(I)nnc21 ; Cn1nc(-c2ccccc2)c2c(Cl)c(C3=CCCC3)nnc21 ; Cn1nc(-c2ccccc2)c2c(Cl)c(-c3ccsc3)nnc21 ; Cn1nc(-c2ccccc2)c2c(Cl)c(-c3cccnc3)nnc21]

or a pharmaceutically acceptable salt thereof.

Pyrazolopyridazine Compounds of Formula XIII

The invention further provides methods for treating or preventing a proteopathy, comprising administering to a subject in need thereof an effective amount of a compound of Formula XIII:

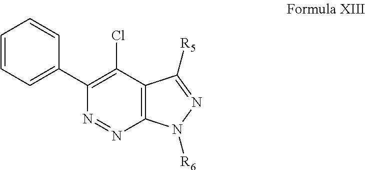

or a pharmaceutically acceptable salt thereof,
wherein R₅ is:

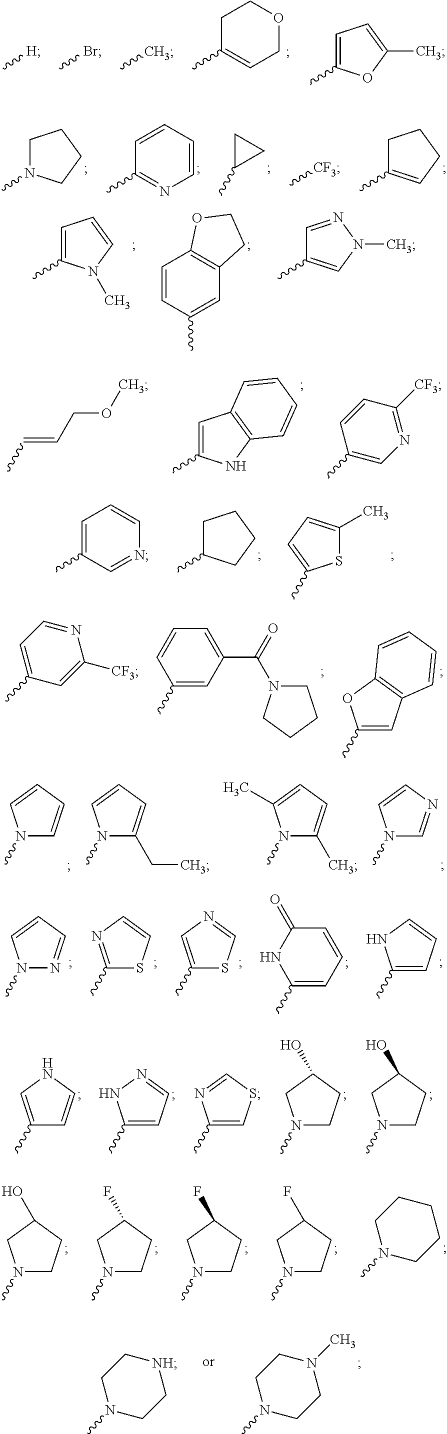

R₆ is:

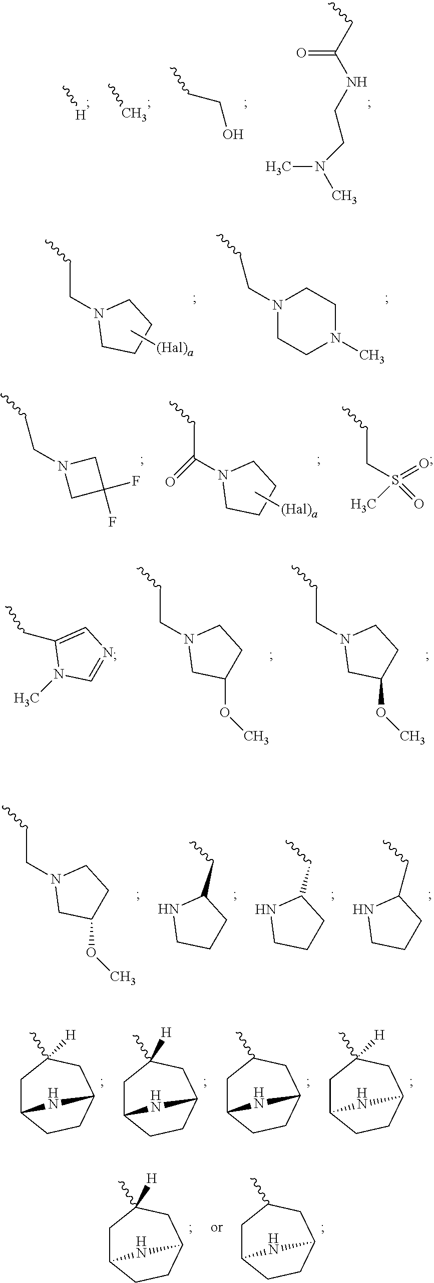

Hal is —Cl, —F, —I, or —Br; and
a is 0, 1, or 2.

In particular embodiments, R₅ is —I. In other embodiments, R₅ is —H. In yet other embodiments, R₅ is —CH₃. In certain embodiments, R₅ is —CF₃.

In yet other embodiments, R₅ is

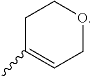

In certain embodiments, R₅ is

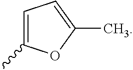

In still further embodiments, R₅ is

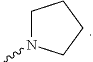

In particular embodiments, R₅ is

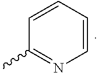

In other embodiments, R₅ is

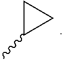

In yet other embodiments, R₅ is

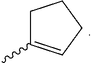

In certain embodiments, R₅ is

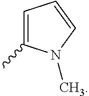

In particular embodiments, R₅ is

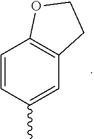

In certain embodiments, R₅ is

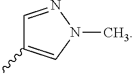

In still further embodiments, R₅ is

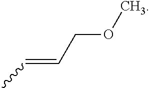

In other embodiments, R₅ is

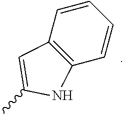

In yet other embodiments, R₅ is

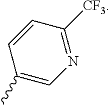

In certain embodiments, R₅ is

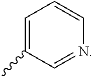

In still further embodiments, R₅ is

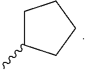

In other embodiments, R₅ is

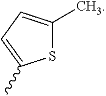

In particular embodiments, R₅ is

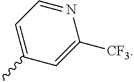

In further embodiments, R₅ is

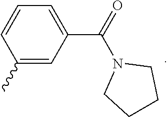

In still further embodiments, R₅ is

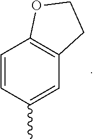

In certain embodiments, R₅ is

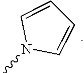

In further embodiments, R₅ is

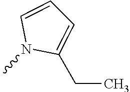

In further embodiments, R₅ is

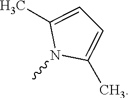

In other embodiments, R₅ is

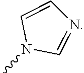

In yet other embodiments, R₅ is

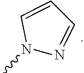

In particular embodiments, R₅ is

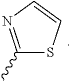

In further embodiments, R₅ is

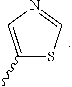

In still further embodiments, R₅ is

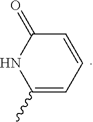

In certain embodiments, R₅ is

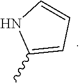

In other embodiments, R₅ is

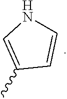

In yet other embodiments, R₅ is

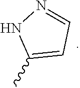

In particular embodiments, R₅ is

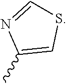

In further embodiments, R₅ is

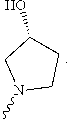

In still further embodiments, R₅ is

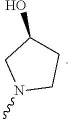

In certain embodiments, R₅ is

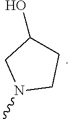

In other embodiments, R₅ is

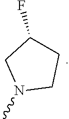

In yet other embodiments, R₅ is

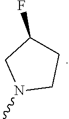

In particular embodiments, R₅ is

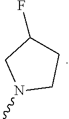

In further embodiments, R₅ is

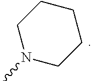

In still further embodiments, R₅ is

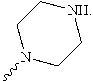

In certain embodiments, R₅ is

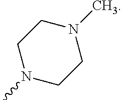

In certain embodiments, R₆ is

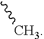

In further embodiments, R₆ is

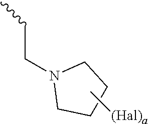

and a=0. In other embodiments, the Pyrazolopyridazine compound of Formula XIII is a pharmaceutically acceptable salt and R₆ is

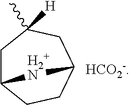

In yet other embodiments, the Pyrazolopyridazine compound of Formula XIII is a pharmaceutically acceptable salt and R₆ is

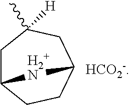

In particular embodiments, the Pyrazolopyridazine compound of Formula XIII is a pharmaceutically acceptable salt and R₆ is

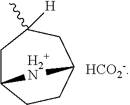

In certain embodiments, the Pyrazolopyridazine compound of Formula XIII is a pharmaceutically acceptable salt and R₆ is

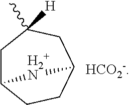

In further embodiments, the Pyrazolopyridazine compound of Formula XIII is a pharmaceutically acceptable salt and R₆ is

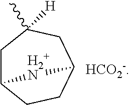

In other embodiments, the Pyrazolopyridazine compound of Formula XIII is a pharmaceutically acceptable salt and R₆ is

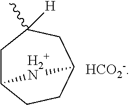

In certain embodiments, R₆ is

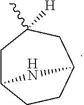

In further embodiments, R₆ is

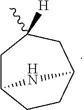

In particular embodiments, R₆ is

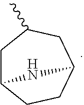

In further embodiments, R₆ is

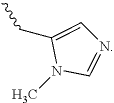

In still further embodiments, R₆ is

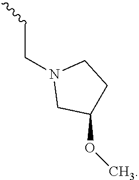

In other embodiments, R₆ is

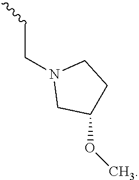

In certain embodiments, R₆ is

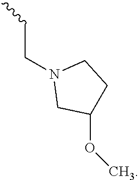

In yet other embodiments, R₆ is

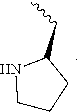

In particular embodiments, R₆ is

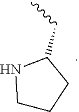

In further embodiments, R₆ is

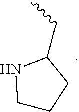

In still further embodiments, R₆ is

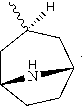

In certain embodiments, R₆ is

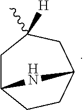

In other embodiments, R₆ is

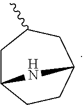

In further embodiments, when a is 2, each Hal is the same or different.

In certain embodiments the compound of Formula XIII has the structure:

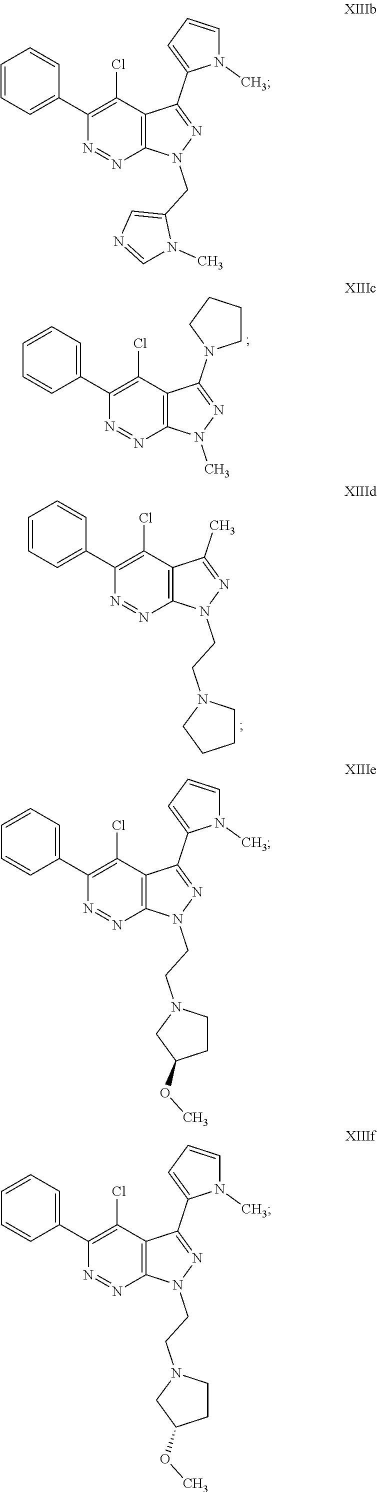

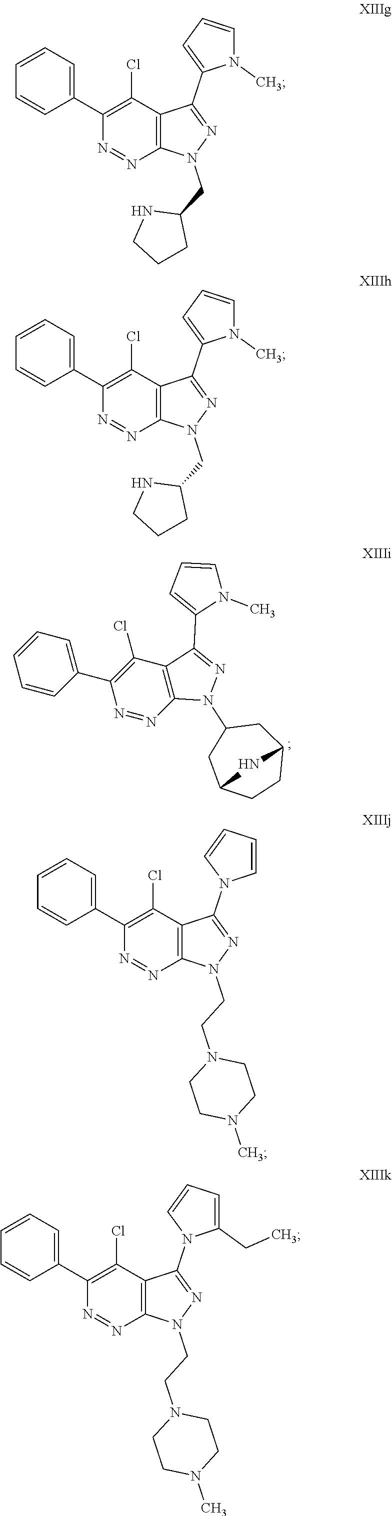

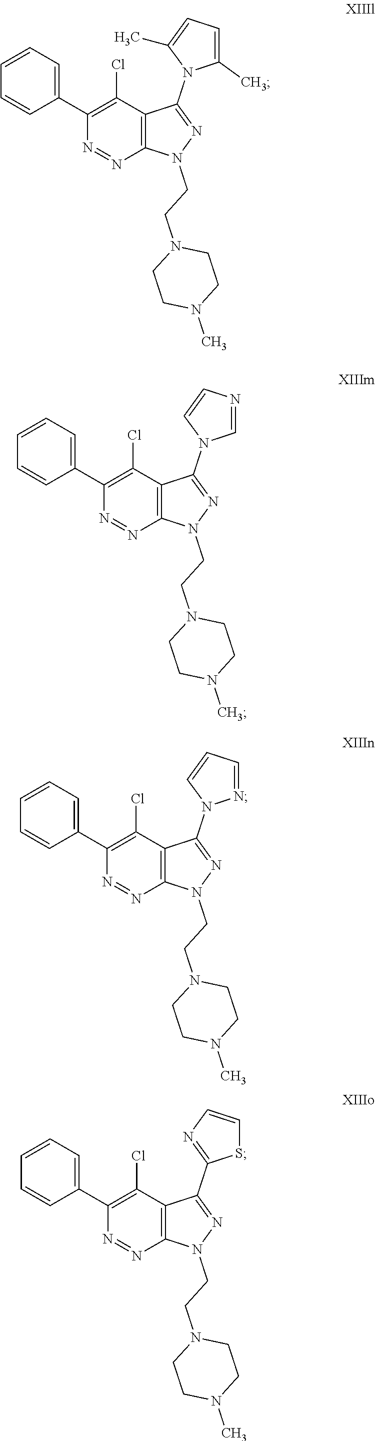

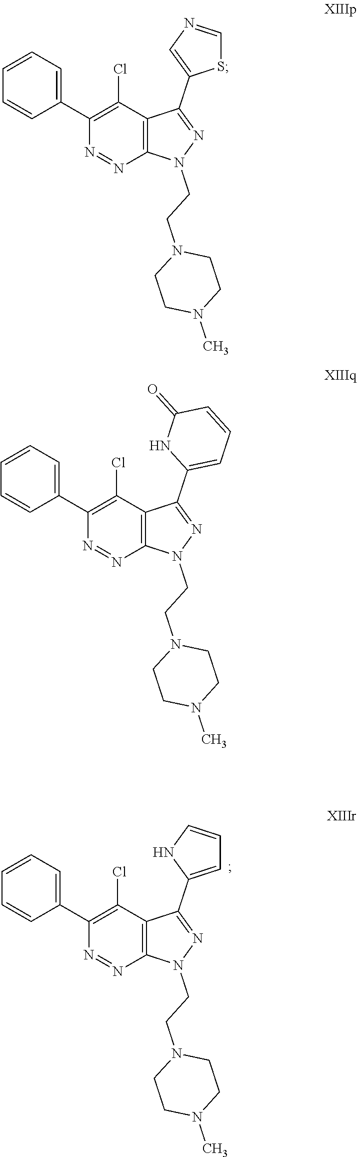 [SMILES: CN1CCN(CCn2nc(-c3cncs3)c3c(Cl)c(-c4ccccc4)nnc32)CC1 ; CN1CCN(CCn2nc(-c3cccc(=O)[nH]3)c3c(Cl)c(-c4ccccc4)nnc32)CC1 ; CN1CCN(CCn2nc(-c3ccc[nH]3)c3c(Cl)c(-c4ccccc4)nnc32)CC1]

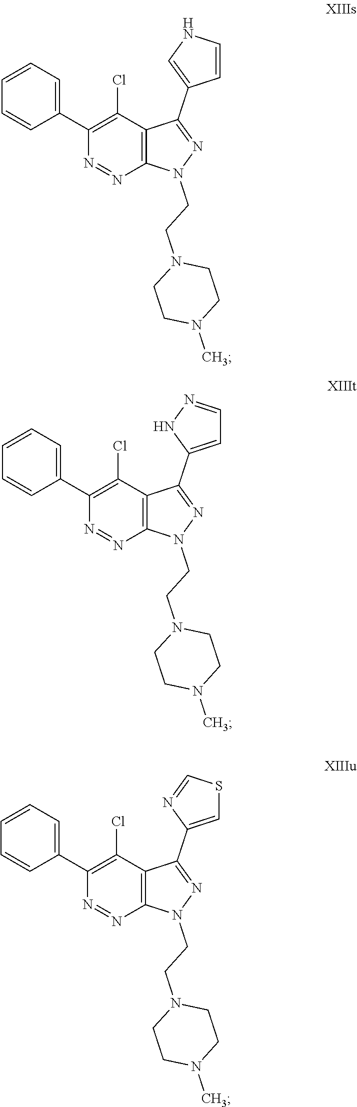

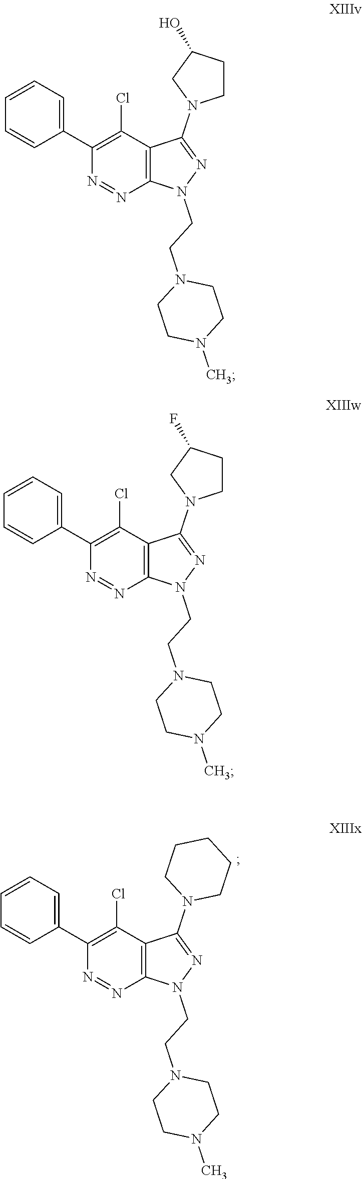 [SMILES: CN1CCN(CCn2nc(N3CC[C@@H](O)C3)c3c(Cl)c(-c4ccccc4)nnc32)CC1 ; CN1CCN(CCn2nc(N3CC[C@@H](F)C3)c3c(Cl)c(-c4ccccc4)nnc32)CC1 ; CN1CCN(CCn2nc(N3CCCCC3)c3c(Cl)c(-c4ccccc4)nnc32)CC1]

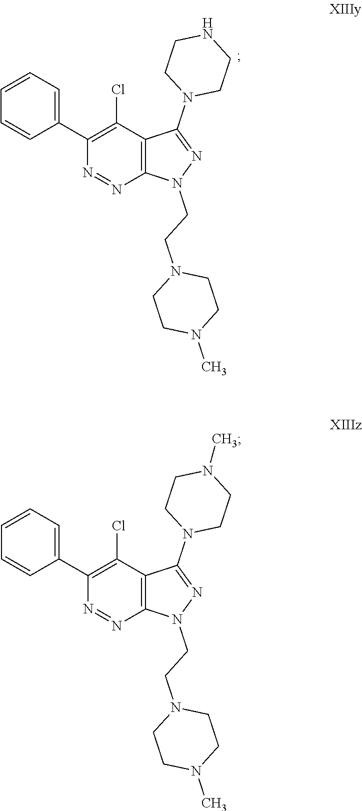 [SMILES: CN1CCN(CCn2nc(N3CCNCC3)c3c(Cl)c(-c4ccccc4)nnc32)CC1 ; CN1CCN(CCn2nc(N3CCN(C)CC3)c3c(Cl)c(-c4ccccc4)nnc32)CC1]

or a pharmaceutically acceptable salt thereof.

In other embodiments, the Pyrazolopyridazine compound of Formula XIII is a pharmaceutically acceptable salt and has the structure:

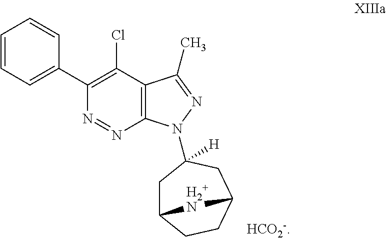

Pyrazolopyridazine Compounds of Formula XIV

The invention also provides methods for treating or preventing a proteopathy, comprising administering to a subject in need thereof an effective amount of a compound of Formula XIV:

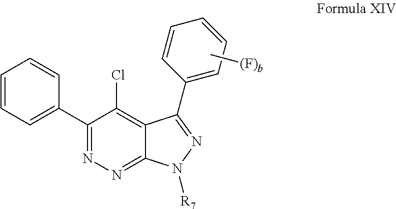

or a pharmaceutically acceptable salt thereof,
wherein R₇ is:

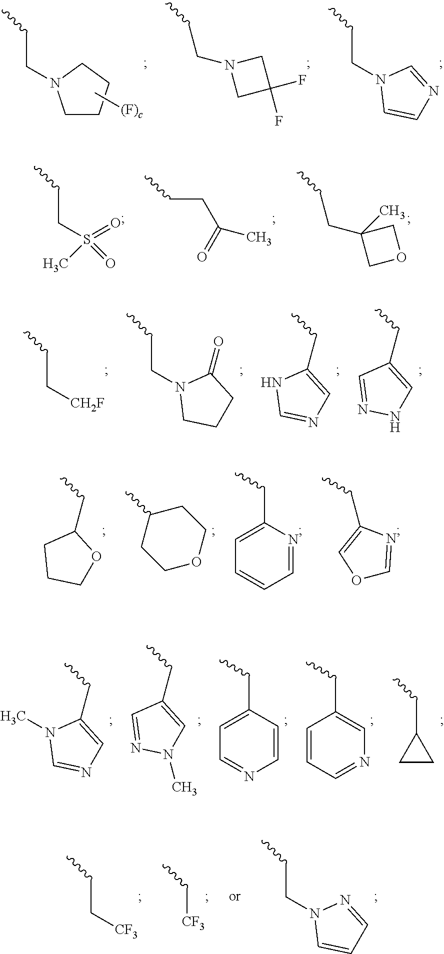

b is 0 or 1; and
c is 1 or 2.

In particular embodiments, b is 0. In other embodiments b is 1 and the —F is in the meta position relative to the pyrazolopyridazino ring system. In yet other embodiments b is 1 and the —F is in the para position relative to the pyrazolopyridazino ring system.

In particular embodiments R₇ is —CF₃. In certain embodiments R₇ is

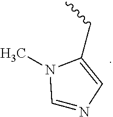

In other embodiments R₇ is

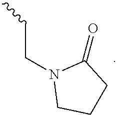

In yet other embodiments R₇ is

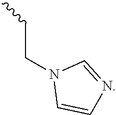

In further embodiments R₇ is

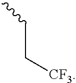

In still further embodiments R₇ is

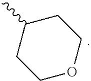

In particular embodiments R₇ is

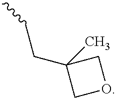

In other embodiments R₇ is

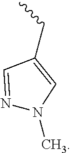

In yet other embodiments R₇ is

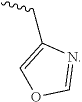

In certain embodiments R₇ is

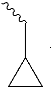

In further embodiments R₇ is

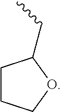

In further embodiments R₇ is

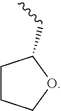

In certain embodiments R₇ is

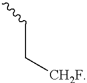

In other embodiments R₇ is

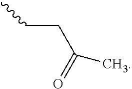

In yet other embodiments R₇ is

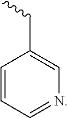

In further embodiments R₇ is

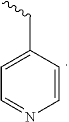

In still further embodiments R₇ is

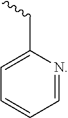

In particular embodiments R₇ is

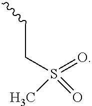

In certain embodiments R₇ is

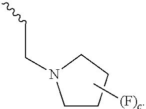

In further embodiments R₇ is

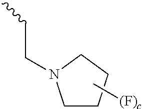

and c=1. In still further embodiments R₇ is

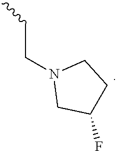

In particular embodiments R₇ is

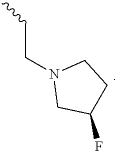

In other embodiments R₇ is

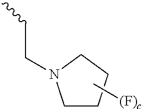

and c=2. In yet other embodiments R₇ is

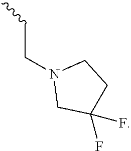

In certain embodiments R₇ is

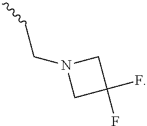

In other embodiments R₇ is

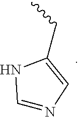

In yet other embodiments R₇ is

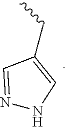

In yet other embodiments R₇ is

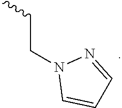

In certain embodiments the compound of Formula XIV has the structure:

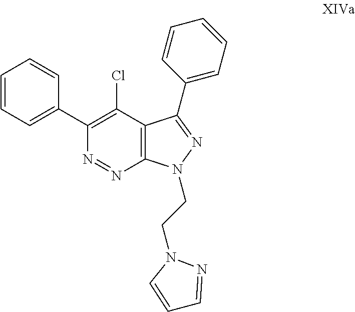 [SMILES: Clc1c(-c2ccccc2)nnc2c1c(-c1ccccc1)nn2CCn1cccn1]

or a pharmaceutically acceptable salt thereof.

Pyrazolopyridazine Compounds of Formula XV

The invention also provides methods for treating or preventing a proteopathy, comprising administering to a subject in need thereof an effective amount of a compound of Formula XV:

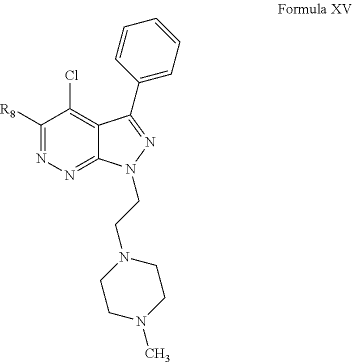

or a pharmaceutically acceptable salt thereof,
wherein R₈ is:

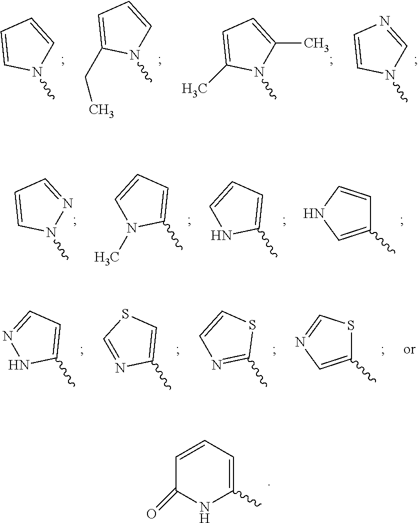

In particular embodiments R₈ is

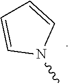

In certain embodiments R₈ is

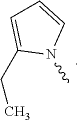

In other embodiments R₈ is

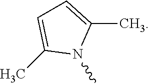

In yet other embodiments R₈ is

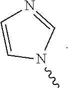

In further embodiments R₈ is

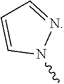

In still further embodiments R₈ is

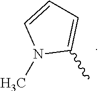

In particular embodiments R₈ is

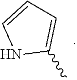

In particular embodiments R₈ is

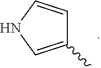

In certain embodiments R₈ is

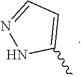

In other embodiments R₈ is

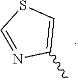

In yet other embodiments R₈ is

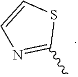

In further embodiments R₈ is

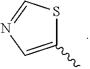

In still further embodiments R₈ is

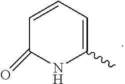

In certain embodiments the compound of Formula XV has the structure:

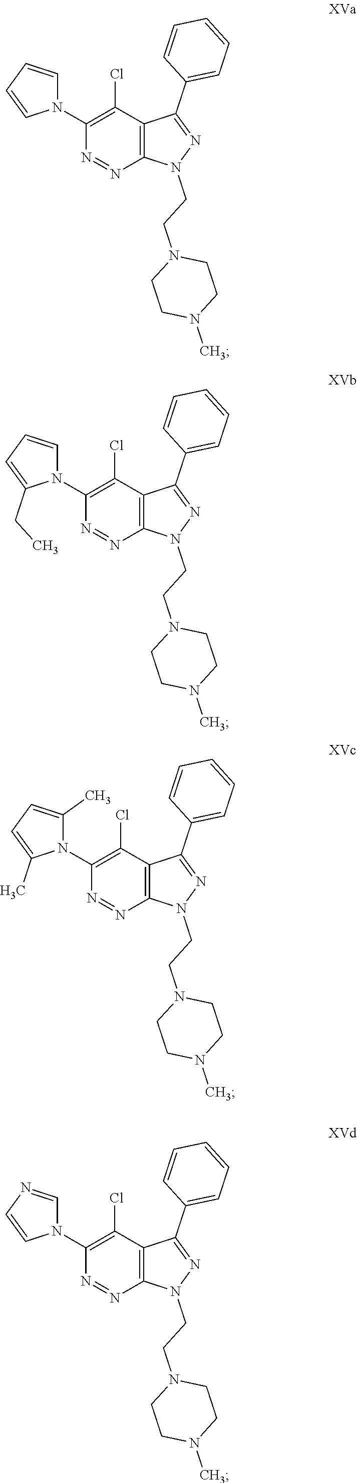

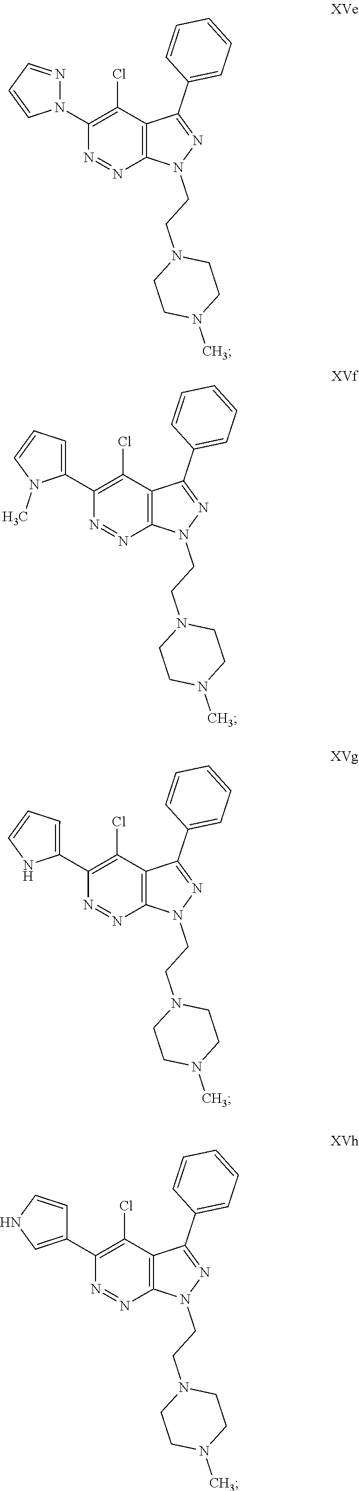

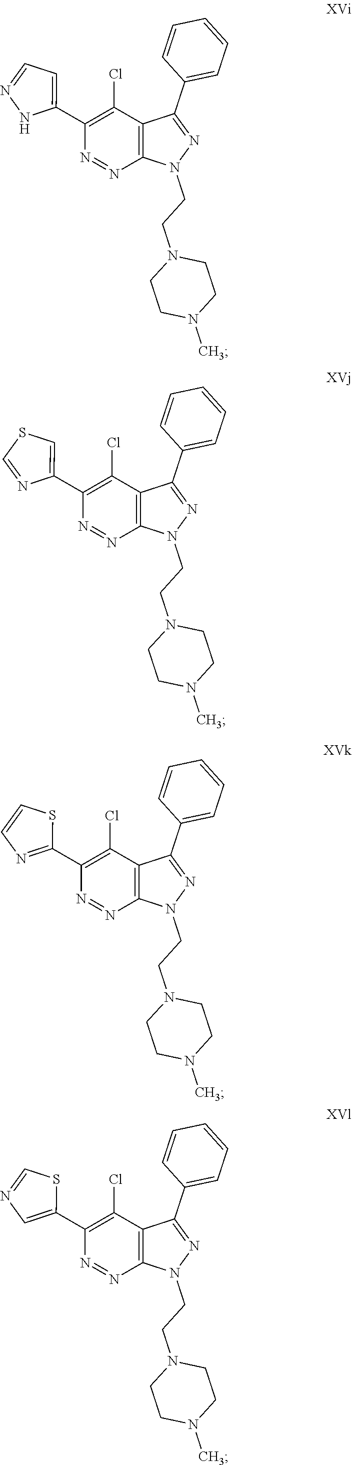

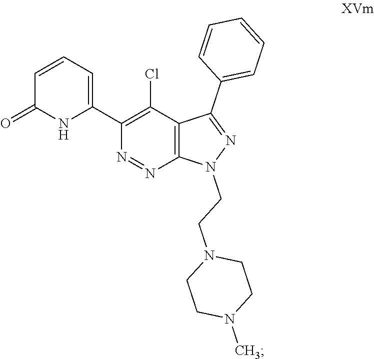 [SMILES: CN1CCN(CCn2nc(-c3ccccc3)c3c(Cl)c(-c4cccc(=O)[nH]4)nnc32)CC1]

or a pharmaceutically acceptable salt thereof.

Other Pyrazolopyridazine Compounds

The invention additionally provides methods for treating or preventing a proteopathy, comprising administering to a subject in need thereof an effective amount of a compound having the structure:

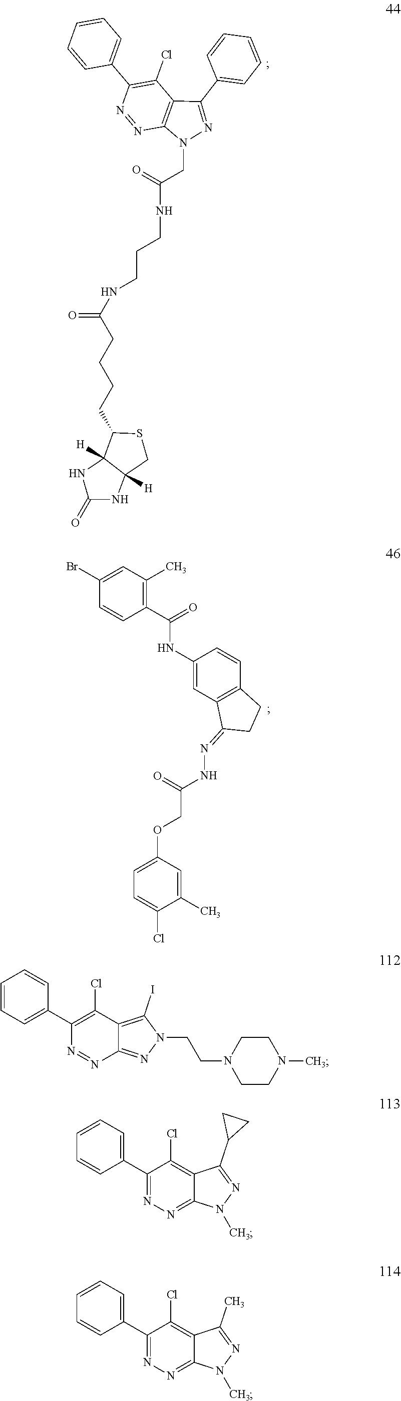

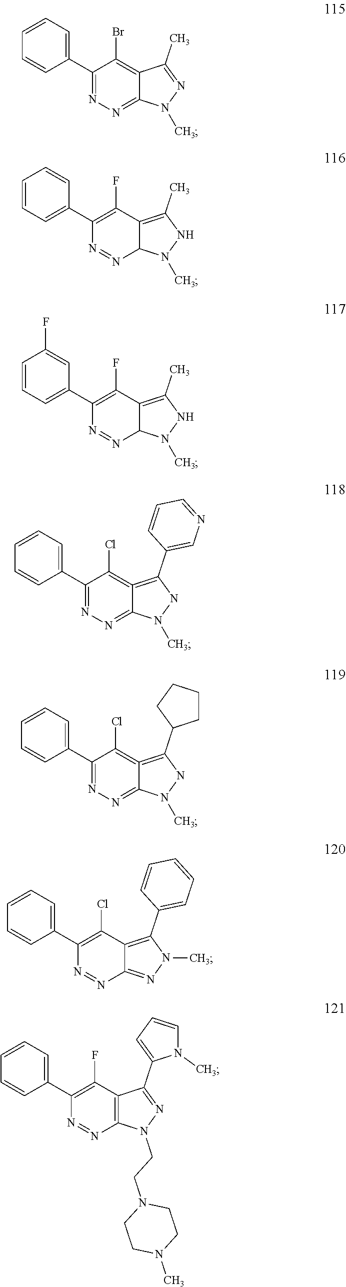

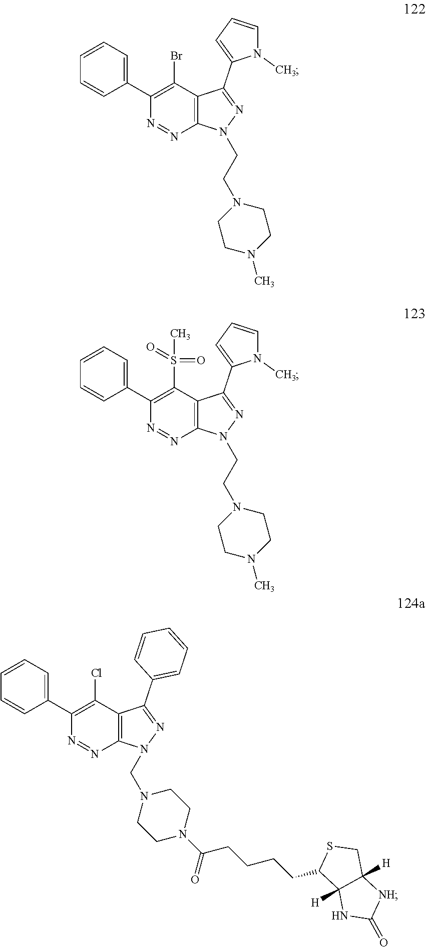

or a pharmaceutically acceptable salt thereof.

Non-Pyrazolopyridazine Compounds

A compound or pharmaceutically acceptable salt of the compound of Table 1 below is a non-Pyrazolopyridazine compound.

In one embodiment, the invention provides non-Pyrazolopyridazine compounds. In a further embodiment, the invention provides pharmaceutical compositions comprising an effective amount of a non-Pyrazolopyridazine compound and a pharmaceutically acceptable carrier or vehicle.

The invention additionally provides methods for treating or preventing a proteopathy, comprising administering to a subject in need thereof an effective amount of a non-Pyrazolopyridazine compound.

TABLE 1
Non-Pyrazolopyridazine compounds

| # | Structure |
|---|---|
| 125 | 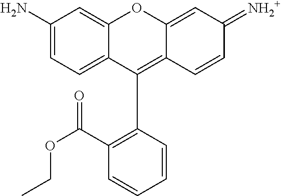 [SMILES: CCOC(=O)c1ccccc1-c1c2ccc(=[NH2+])cc-2oc2cc(N)ccc12] |
| 126 | 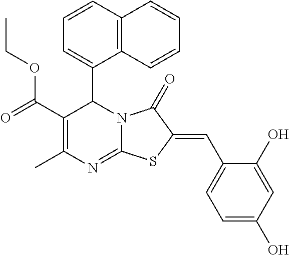 [SMILES: CCOC(=O)C1=C(C)N=c2s/c(=C\c3ccc(O)cc3O)c(=O)n2C1c1cccc2ccccc12] |
| 127 | 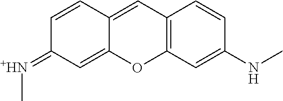 [SMILES: CNc1ccc2cc3cc/c(=[NH+]/C)cc-3oc2c1] |
| 128 | 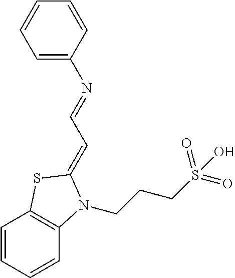 [SMILES: O=S(=O)(O)CCCN1/C(=C/C=N/c2ccccc2)Sc2ccccc21] |
| 129 | 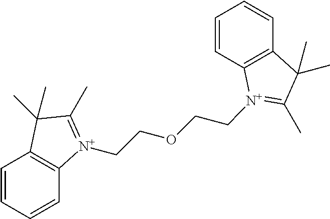 [SMILES: CC1=[N+](CCOCC[N+]2=C(C)C(C)(C)c3ccccc32)c2ccccc2C1(C)C] |
| 130 | 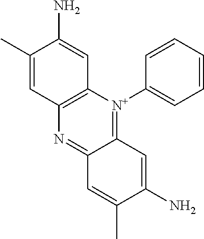 [SMILES: Cc1cc2nc3cc(C)c(N)cc3[n+](-c3ccccc3)c2cc1N] |
| 131 | 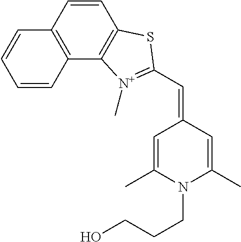 [SMILES: CC1=CC(=Cc2sc3ccc4ccccc4c3[n+]2C)C=C(C)N1CCCO] |
| 132 | 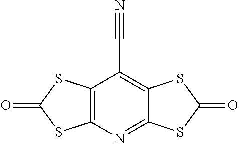 [SMILES: N#Cc1c2sc(=O)sc2nc2sc(=O)sc12] |
| 133 | 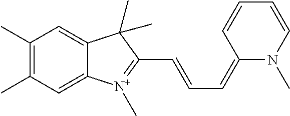 |
| 134 | 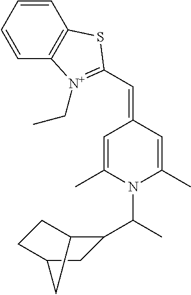 [SMILES: CC[n+]1c(C=C2C=C(C)N(C(C)C3CC4CCC3C4)C(C)=C2)sc2ccccc21] |
| 135 | 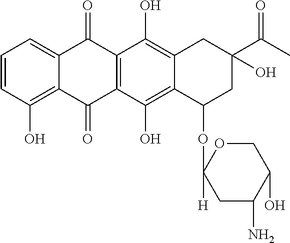 [SMILES: CC(=O)C1(O)Cc2c(O)c3c(c(O)c2C(OC2CC(N)C(O)CO2)C1)C(=O)c1c(O)cccc1C3=O] |
| 136 | 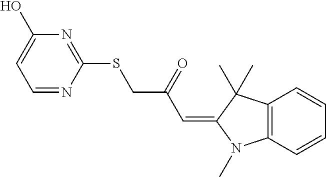 [SMILES: CN1/C(=C/C(=O)CSc2nccc(O)n2)C(C)(C)c2ccccc21] |
| 137 | 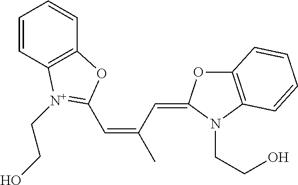 [SMILES: CC(=C/c1oc2ccccc2[n+]1CCO)/C=C1/Oc2ccccc2N1CCO] |
| 138 | 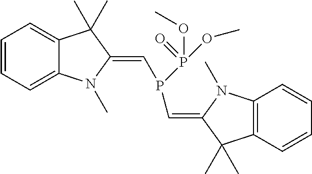 [SMILES: COP(=O)(OC)P(/C=C1\N(C)c2ccccc2C1(C)C)/C=C1\N(C)c2ccccc2C1(C)C] |
| 139 | 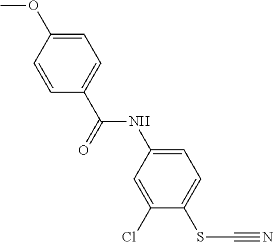 [SMILES: COc1ccc(C(=O)Nc2ccc(SC#N)c(Cl)c2)cc1] |
| 140 | 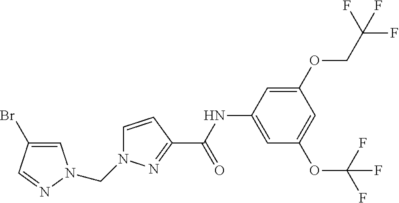 [SMILES: O=C(Nc1cc(OCC(F)(F)F)cc(OC(F)(F)F)c1)c1ccn(Cn2cc(Br)cn2)n1] |
| 141 | 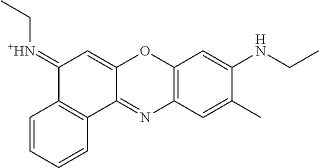 [SMILES: CCNc1cc2oc3c/c(=[NH+]\CC)c4ccccc4c-3nc2cc1C] |
| 142 | 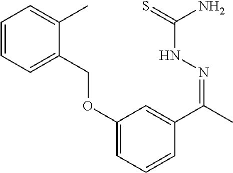 [SMILES: C/C(=N/NC(N)=S)c1cccc(OCc2ccccc2C)c1] |
| 143 | 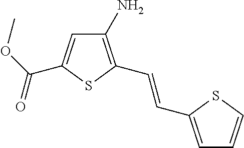 [SMILES: COC(=O)c1cc(N)c(/C=C/c2cccs2)s1] |
| 144 | 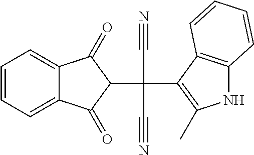 [SMILES: Cc1[nH]c2ccccc2c1C(C#N)(C#N)C1C(=O)c2ccccc2C1=O] |
| 145 | 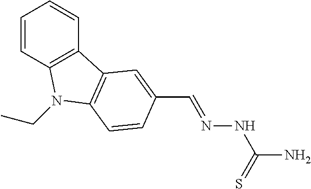 [SMILES: CCn1c2ccccc2c2cc(/C=N/NC(N)=S)ccc21] |
| 146 | 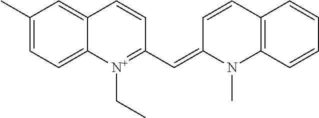 [SMILES: CC[n+]1c(/C=C2\C=Cc3ccccc3N2C)ccc2cc(C)ccc21] |
| 147 | 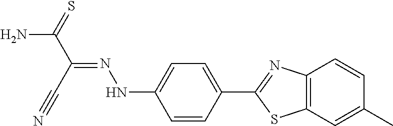 [SMILES: Cc1ccc2nc(-c3ccc(N/N=C(\C#N)C(N)=S)cc3)sc2c1] |
| 148 | 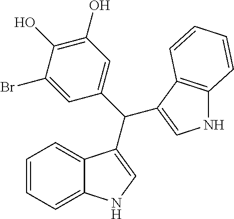 [SMILES: Oc1cc(C(c2c[nH]c3ccccc23)c2c[nH]c3ccccc23)cc(Br)c1O] |
| 149 | 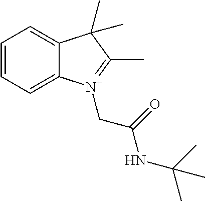 [SMILES: CC1=[N+](CC(=O)NC(C)(C)C)c2ccccc2C1(C)C] |
| 150 | 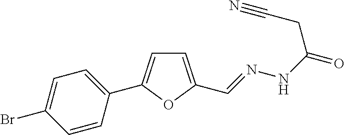 [SMILES: N#CCC(=O)N/N=C/c1ccc(-c2ccc(Br)cc2)o1] |
| 151 | 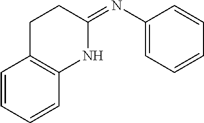 [SMILES: c1ccc(/N=C2/CCc3ccccc3N2)cc1] |
| 152 | 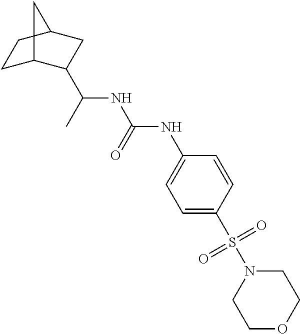 [SMILES: CC(NC(=O)Nc1ccc(S(=O)(=O)N2CCOCC2)cc1)C1CC2CCC1C2] |
| 153 | 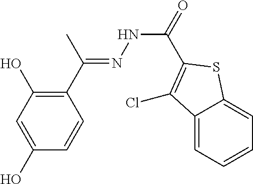 [SMILES: C/C(=N\NC(=O)c1sc2ccccc2c1Cl)c1ccc(O)cc1O] |
| 154 | 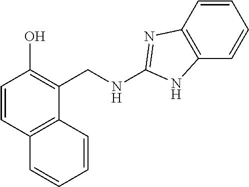 [SMILES: Oc1ccc2ccccc2c1CNc1nc2ccccc2[nH]1] |
| 155 | 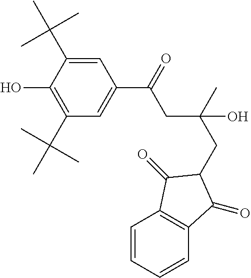 [SMILES: CC(O)(CC(=O)c1cc(C(C)(C)C)c(O)c(C(C)(C)C)c1)CC1C(=O)c2ccccc2C1=O] |
| 156 | 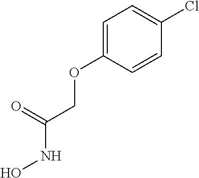 [SMILES: O=C(COc1ccc(Cl)cc1)NO] |
| 157 | 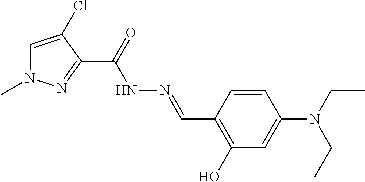 [SMILES: CCN(CC)c1ccc(/C=N/NC(=O)c2nn(C)cc2Cl)c(O)c1] |
| 158 | 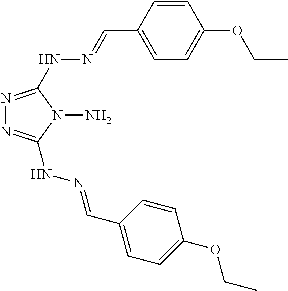 [SMILES: CCOc1ccc(/C=N/Nc2nnc(N/N=C/c3ccc(OCC)cc3)n2N)cc1] |
| 159 | 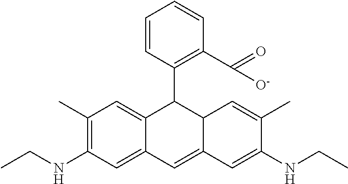 [SMILES: CCNC1=CC2=Cc3cc(NCC)c(C)cc3C(c3ccccc3C(=O)[O-])C2C=C1C] |
| 160 | 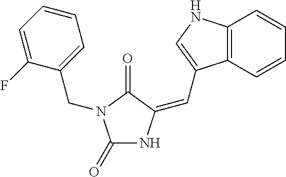 [SMILES: O=C1N/C(=C/c2c[nH]c3ccccc23)C(=O)N1Cc1ccccc1F] |
| 161 | 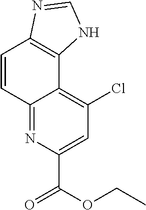 [SMILES: CCOC(=O)c1cc(Cl)c2c(ccc3nc[nH]c32)n1] |
| 162 | 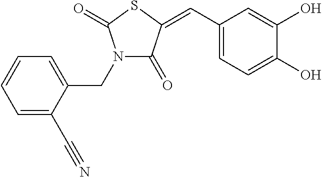 [SMILES: N#Cc1ccccc1CN1C(=O)S/C(=C/c2ccc(O)c(O)c2)C1=O] |
| 163 | 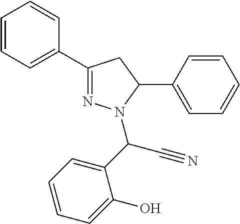 [SMILES: N#CC(c1ccccc1O)N1N=C(c2ccccc2)CC1c1ccccc1] |
| 164 | 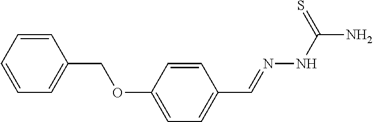 [SMILES: NC(=S)N/N=C/c1ccc(OCc2ccccc2)cc1] |
| 165 | 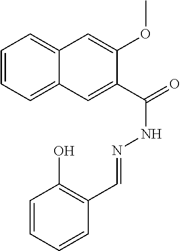 [SMILES: COc1cc2ccccc2cc1C(=O)N/N=C/c1ccccc1O] |
| 166 | 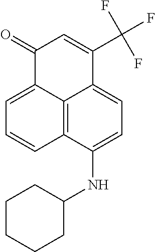 [SMILES: O=C1C=C(C(F)(F)F)c2ccc(NC3CCCCC3)c3cccc1c23] |
| 167 | 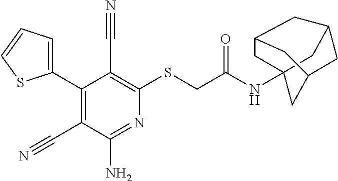 [SMILES: N#Cc1c(N)nc(SCC(=O)NC23CC4CC(CC(C4)C2)C3)c(C#N)c1-c1cccs1] |
| 168 | 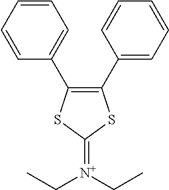 [SMILES: CC[N+](CC)=c1sc(-c2ccccc2)c(-c2ccccc2)s1] |
| 169 | 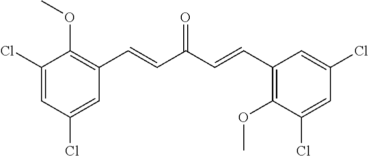 [SMILES: COc1c(Cl)cc(Cl)cc1/C=C/C(=O)/C=C/c1cc(Cl)cc(Cl)c1OC] |
| 170 | 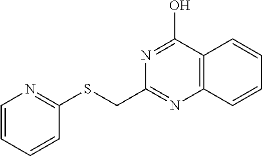 [SMILES: Oc1nc(CSc2ccccn2)nc2ccccc12] |
| 171 | 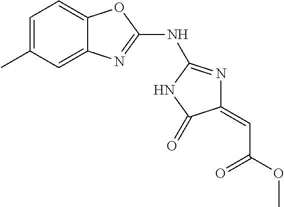 |
| 172 | 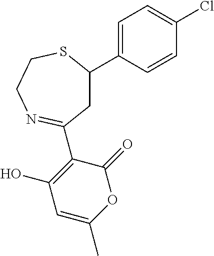 [SMILES: Cc1cc(O)c(C2=NCCSC(c3ccc(Cl)cc3)C2)c(=O)o1] |
| 173 | 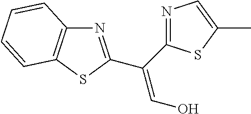 [SMILES: Cc1cnc(/C(=C\O)c2nc3ccccc3s2)s1] |
| 174 | 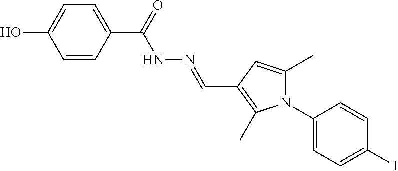 [SMILES: Cc1cc(/C=N/NC(=O)c2ccc(O)cc2)c(C)n1-c1ccc(I)cc1] |
| 175 | 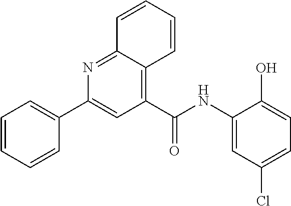 [SMILES: O=C(Nc1cc(Cl)ccc1O)c1cc(-c2ccccc2)nc2ccccc12] |
| 176 | 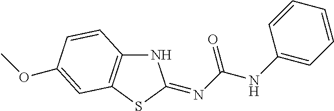 [SMILES: COc1ccc2[nH]/c(=N\C(=O)Nc3ccccc3)sc2c1] |
| 177 | 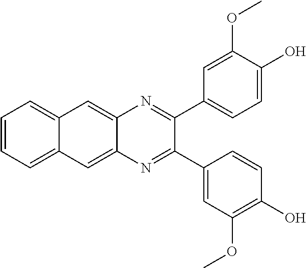 [SMILES: COc1cc(-c2nc3cc4ccccc4cc3nc2-c2ccc(O)c(OC)c2)ccc1O] |
| 178 | 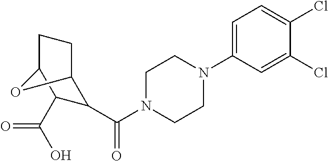 [SMILES: O=C(O)C1C2CCC(O2)C1C(=O)N1CCN(c2ccc(Cl)c(Cl)c2)CC1] |
| 179 | 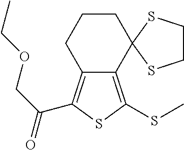 [SMILES: CCOCC(=O)c1sc(SC)c2c1CCCC21SCCS1] |
| 180 | 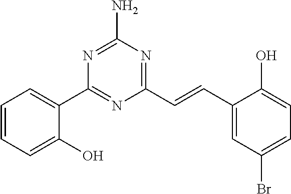 [SMILES: Nc1nc(/C=C/c2cc(Br)ccc2O)nc(-c2ccccc2O)n1] |
| 181 | 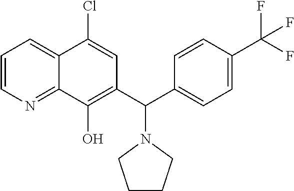 [SMILES: Oc1c(C(c2ccc(C(F)(F)F)cc2)N2CCCC2)cc(Cl)c2cccnc12] |
| 182 | 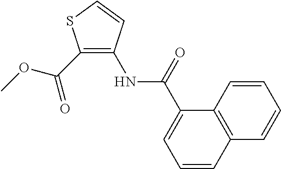 [SMILES: COC(=O)c1sccc1NC(=O)c1cccc2ccccc12] |
| 183 | 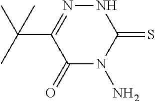 |
| 184 | 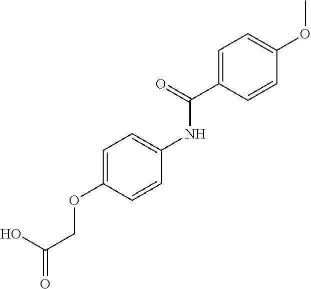 [SMILES: COc1ccc(C(=O)Nc2ccc(OCC(=O)O)cc2)cc1] |
| 185 | 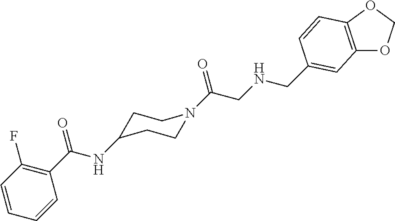 [SMILES: O=C(NC1CCN(C(=O)CNCc2ccc3c(c2)OCO3)CC1)c1ccccc1F] |
| 186 | 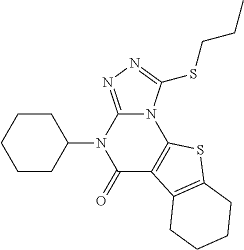 [SMILES: CCCSc1nnc2n(C3CCCCC3)c(=O)c3c4c(sc3n12)CCCC4] |
| 187 | 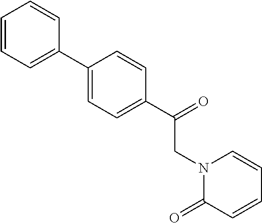 [SMILES: O=C(Cn1ccccc1=O)c1ccc(-c2ccccc2)cc1] |
| 188 | 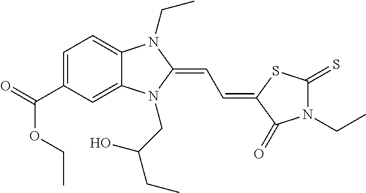 [SMILES: CCOC(=O)c1ccc2c(c1)N(CC(O)CC)/C(=C\C=C1/SC(=S)N(CC)C1=O)N2CC] |
| 189 | 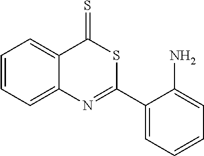 [SMILES: Nc1ccccc1-c1nc2ccccc2c(=S)s1] |
| 190 | 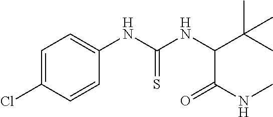 [SMILES: CNC(=O)C(NC(=S)Nc1ccc(Cl)cc1)C(C)(C)C] |
| 191 | 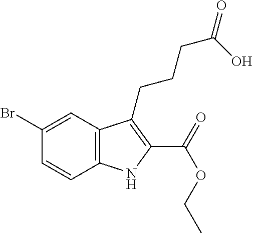 [SMILES: CCOC(=O)c1[nH]c2ccc(Br)cc2c1CCCC(=O)O] |
| 192 | 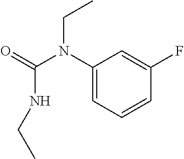 |
| 193 | 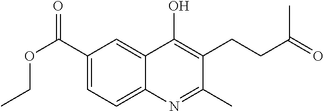 [SMILES: CCOC(=O)c1ccc2nc(C)c(CCC(C)=O)c(O)c2c1] |
| 194 | 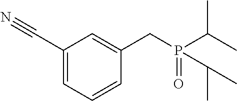 [SMILES: CC(C)P(=O)(Cc1cccc(C#N)c1)C(C)C] |
| 195 | 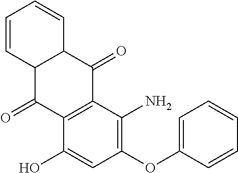 [SMILES: Nc1c(Oc2ccccc2)cc(O)c2c1C(=O)C1C=CC=CC1C2=O] |
| 196 | 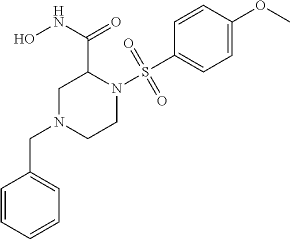 [SMILES: COc1ccc(S(=O)(=O)N2CCN(Cc3ccccc3)CC2C(=O)NO)cc1] |
| 197 | 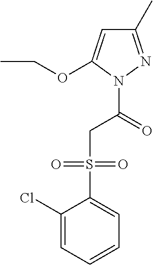 [SMILES: CCOc1cc(C)nn1C(=O)CS(=O)(=O)c1ccccc1Cl] |
| 198 | 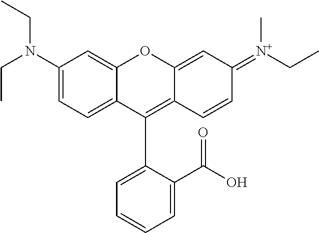 [SMILES: CCN(CC)c1ccc2c(-c3ccccc3C(=O)O)c3cc/c(=[N+](/C)CC)cc-3oc2c1] |
| 199 | 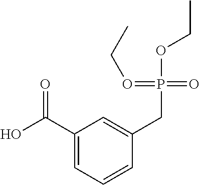 |
| 200 | 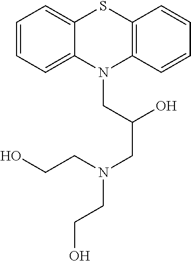 [SMILES: OCCN(CCO)CC(O)CN1c2ccccc2Sc2ccccc21] |
| 201 | 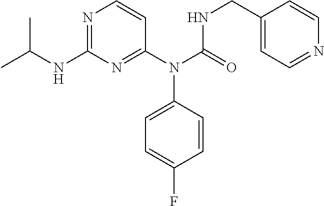 [SMILES: CC(C)Nc1nccc(N(C(=O)NCc2ccncc2)c2ccc(F)cc2)n1] |
| 202 | 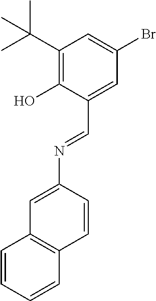 [SMILES: CC(C)(C)c1cc(Br)cc(/C=N/c2ccc3ccccc3c2)c1O] |
| 203 | 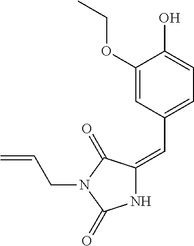 [SMILES: C=CCN1C(=O)N/C(=C/c2ccc(O)c(OCC)c2)C1=O] |
| 204 | 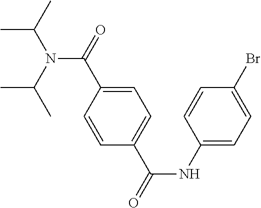 [SMILES: CC(C)N(C(=O)c1ccc(C(=O)Nc2ccc(Br)cc2)cc1)C(C)C] |
| 205 | 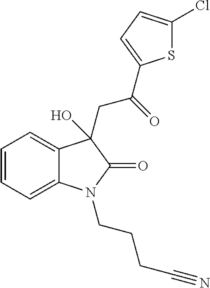 [SMILES: N#CCCCN1C(=O)C(O)(CC(=O)c2ccc(Cl)s2)c2ccccc21] |
| 206 | 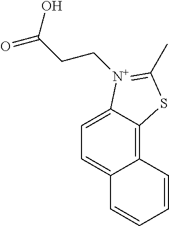 [SMILES: Cc1sc2c3ccccc3ccc2[n+]1CCC(=O)O] |
| 207 | 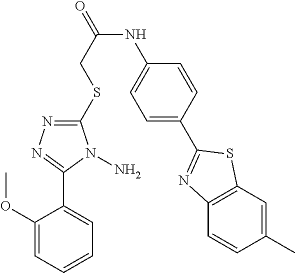 [SMILES: COc1ccccc1-c1nnc(SCC(=O)Nc2ccc(-c3nc4ccc(C)cc4s3)cc2)n1N] |
| 208 | 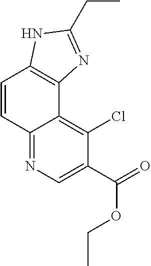 [SMILES: CCOC(=O)c1cnc2ccc3[nH]c(CC)nc3c2c1Cl] |
| 209 | 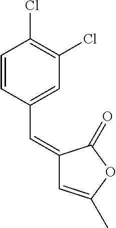 [SMILES: CC1=C/C(=C/c2ccc(Cl)c(Cl)c2)C(=O)O1] |
| 210 | 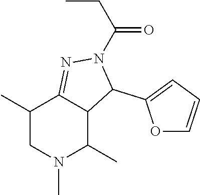 [SMILES: CCC(=O)N1N=C2C(C)CN(C)C(C)C2C1c1ccco1] |
| 211 | 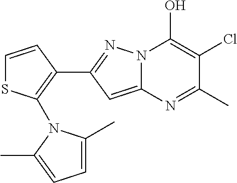 |
| 212 | 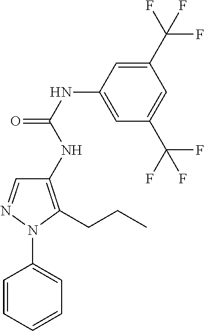 [SMILES: CCCc1c(NC(=O)Nc2cc(C(F)(F)F)cc(C(F)(F)F)c2)cnn1-c1ccccc1] |
| 213 | 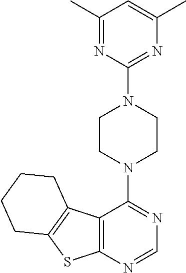 [SMILES: Cc1cc(C)nc(N2CCN(c3ncnc4sc5c(c34)CCCC5)CC2)n1] |

Some of the compounds disclosed herein, for example, Compounds 44, 63, 72, 74, 83, 88, 89, 98-101, 111, 124a, 124b, Vp, Vq, Vt, VIj, VIt, VIu, VIx, VIy, XIIIa, XIIIe, XIIIf, XIIIg, XIIIh, XIIIi, XIIIv, and XIIIw are depicted having a bold or hatched wedge, indicating absolute stereochemistry.

The Pyrazolopyridazine compounds as well as the non-Pyrazolopyridazine compounds can be in the form of a salt. In some embodiments, the salt is a pharmaceutically acceptable salt. Pharmaceutically acceptable salts include, for example, acid-addition salts and base-addition salts. The acid that forms an acid-addition salt can be an organic acid or an inorganic acid. A base that forms a base-addition salt can be an organic base or an inorganic base. In some embodiments, a pharmaceutically acceptable salt is a metal salt. In some embodiments, a pharmaceutically acceptable salt is an ammonium salt.

Acid-addition salts can arise from the addition of an acid to the free-base form of a Pyrazolopyridazine compound or non-Pyrazolopyridazine compound. In some embodiments, the acid is organic. In some embodiments, the acid is inorganic. Non-limiting examples of suitable acids include hydrochloric acid, hydrobromic acid, hydroiodic acid, nitric acid, nitrous acid, sulfuric acid, sulfurous acid, a phosphoric acid, nicotinic acid, isonicotinic acid, lactic acid, salicylic acid, 4-aminosalicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, citric acid, oxalic acid, maleic acid, hydroxymaleic acid, methylmaleic acid, glycolic acid, malic acid, cinnamic acid, mandelic acid, 2-phenoxybenzoic acid, 2-acetoxybenzoic acid, embonic acid, phenylacetic acid, N-cyclohexylsulfamic acid, methanesulfonic acid, ethanesulfonic acid, benzenesulfonic acid, p-toluenesulfonic acid, 2-hydroxyethanesulfonic acid, ethane-1,2-disulfonic acid, 4-methylbenzenesulfonic acid, naphthalene-2-sulfonic acid, naphthalene-1,5-disulfonic acid, 2-phosphoglyceric acid, 3-phosphoglyceric acid, glucose-6-phosphoric acid, and an amino acid.

Non-limiting examples of suitable acid-addition salts include a hydrochloride salt, a hydrobromide salt, a hydroiodide salt, a nitrate salt, a nitrite salt, a sulfate salt, a sulfite salt, a phosphate salt, a hydrogen phosphate salt, a dihydrogen phosphate salt, a carbonate salt, a bicarbonate salt, a nicotinate salt, an isonicotinate salt, a lactate salt, a salicylate salt, a 4-aminosalicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a citrate salt, an oxalate salt, a maleate salt, a hydroxymaleate salt, a methylmaleate salt, a glycolate salt, a malate salt, a cinnamate salt, a mandelate salt, a 2-phenoxybenzoate salt, a 2-acetoxybenzoate salt, an embonate salt, a phenylacetate salt, an N-cyclohexylsulfamate salt, a methanesulfonate salt, an ethanesulfonate salt, a benzenesulfonate salt, a p-toluenesulfonate salt, a 2-hydroxyethanesulfonate salt, an ethane-1,2-disulfonate salt, a 4-methylbenzenesulfonate salt, a naphthalene-2-sulfonate salt, a naphthalene-1,5-disulfonate salt, a 2-phosphoglycerate salt, a 3-phosphoglycerate salt, a glucose-6-phosphate salt, and an amino acid salt.

Metal salts can arise from the addition of an inorganic base to a Pyrazolopyridazine compound or non-Pyrazolopyridazine compound having a carboxyl group. The inorganic base consists of a metal cation paired with a basic couterion, such as, for example, hydroxide, carbonate, bicarbonate, or phosphate. The metal can be an alkali metal, alkaline earth metal, transition metal, or main group metal. Non-limiting examples of suitable metals include lithium, sodium, potassium, cesium, cerium, magnesium, manganese, iron, calcium, strontium, cobalt, titanium, aluminum, copper, cadmium, and zinc.

Non-limiting examples of suitable metal salts include a lithium salt, a sodium salt, a potassium salt, a cesium salt, a cerium salt, a magnesium salt, a manganese salt, an iron salt, a calcium salt, a strontium salt, a cobalt salt, a titanium salt, a aluminum salt, a copper salt, a cadmium salt, and a zinc salt.

Ammonium salts can arise from the addition of ammonia or an organic amine to a Pyrazolopyridazine compound or non-Pyrazolopyridazine compound having a carboxyl group. Non-limiting examples of suitable organic amines include triethyl amine, diisopropyl amine, ethanol amine, diethanol amine, triethanol amine, morpholine, N-methylmorpholine, piperidine, N-methylpiperidine, N-ethylpiperidine, dibenzyl amine, piperazine, pyridine, pyrrazole, imidazole, pyrazine, pipyrazine, ethylenediamine, N,N′-dibenzylethylene diamine, procaine, chloroprocaine, choline, dicyclohexyl amine, and N-methylglucamine.

Non-limiting examples of suitable ammonium salts include is a triethylammonium salt, a diisopropylammonium salt, an ethanolammonium salt, a diethanolammonium salt, a triethanolammonium salt, a morpholinium salt, an N-methylmorpholinium salt, a piperidinium salt, an N-methylpiperidinium salt, an N-ethylpiperidinium salt, a dibenzylammonium salt, a piperazinium salt, a pyridinium salt, a pyrrazolium salt, an imidazolium salt, a pyrazinium salt, an ethylenediammonium salt, an N,N′-dibenzylethylenediammonium salt, a procaine salt, a chloroprocaine salt, a choline salt, a dicyclohexylammonium salt, and a N-methylglucamine salt.

### Methods for Making the Pyrazolopyridazine Compounds

Methods for making the Pyrazolopyridazine compounds are disclosed in U.S. Pat. No. 8,765,762, US 2013/0252936 and US 2014/0121197, each of which is incorporated by reference herein in its entirety.

Compound 114 can be synthesized according to Vasilevsky, S. F. & Tretyakov, E. V. Cinnolines and pyrazolopyridazines.—Novel synthetic and mechanistic aspects of the Richter reaction. Liebigs Annalen 1995, 775-779 (1995).

Non-limiting examples of synthetic schema that are useful for synthesizing the Pyrazolopyridazine compounds include the following.

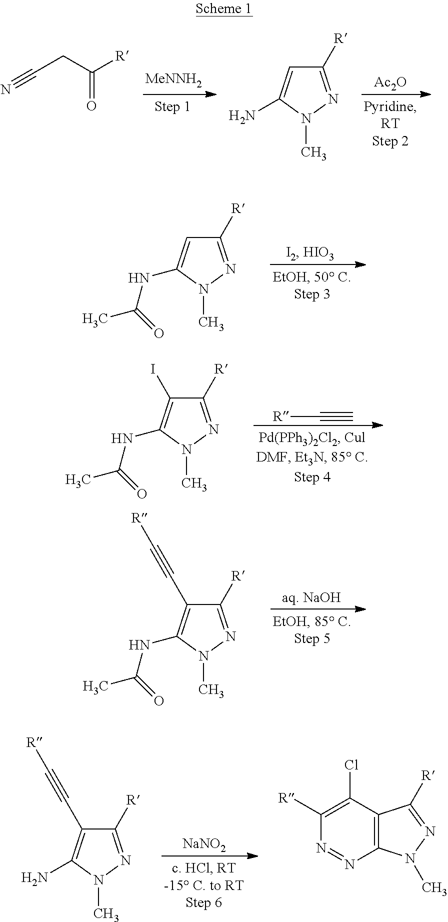

Scheme 1 generally describes the preparation of Pyrazolopyridazine compounds having a 1-N-methyl group and where R′ and R″ are independently an unsubstituted or a substituted phenyl group. For example, a 2-cyanocarbonyl compound in which R′ is unsubstituted or substituted phenyl is condensed with N-methylhydrazine to provide a 3-substituted-1-methyl-1H-pyrazol-5-amine. The 5-amino group is acylated, for example, with acetic anhydride in the presence of a base, such as pyridine, to provide a 5-amido compound. The 5-amido compound is iodinated, for example, with a mixture of iodine and iodic acid in a solvent such as ethanol (EtOH) to provide an N-(3-substituted-4-iodo-1-methyl-1H-pyrazol-5-yl)acetamide. A palladium-mediated cross-coupling, such as a Sonagashira cross-coupling, of the acetamide with an R″-substituted terminal alkyne, catalyzed, for example, by a palladium complex such as palladium (II) bistriphenylphosphine dichloride in the presence of copper (I) iodide in a solvent such as dimethylformamide (DMF) with a base such as triethylamine provides a disubstituted alkyne in which R″ is unsubstituted or substituted phenyl. Saponification of the alkyne acetamide with a base such as sodium hydroxide in a solvent such as ethanol provides the primary amine. Diazotization of the primary amine with sodium nitrite in concentrated hydrochloric acid provides a diazo intermediate, which cyclizes to provide a Pyrazolopyridazine compound having a 1-N-methyl group and where R′ and R″ are independently an unsubstituted or a substituted phenyl group.

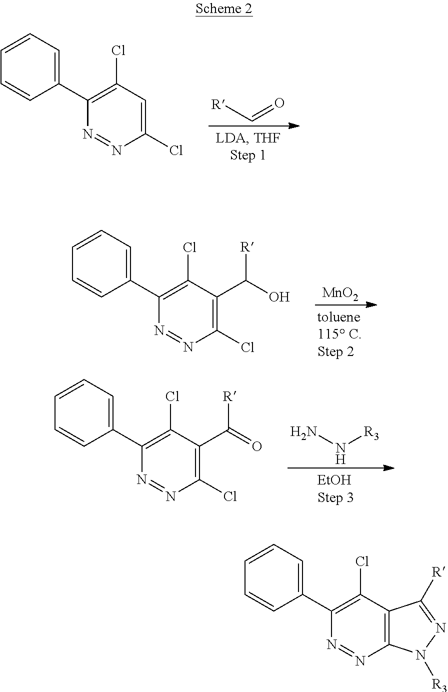

Scheme 2 generally describes the preparation of Pyrazolopyridazine compounds having an R₃ group and in which R′ is an unsubstituted or a substituted phenyl group. R′ and R₃ can be the same or different. For example, 4,6-dichloro-3-phenylpyridazine is deprotonated with a base such as lithium diisopropyl amide (LDA) in a solvent such as tetrahydrofuran (THF), and the resultant 5-lithio species is condensed with an unsubstituted or a substituted benzaldehyde to provide a secondary alcohol. The alcohol is oxidized to a ketone with an oxidizing agent such as manganese dioxide in a solvent such as toluene. The ketone is condensed with an R₃-substituted hydrazine in a solvent such as ethanol to provide an intermediate hydrazone, which cyclizes to provide a Pyrazolopyridazine compound having a 1-N—R₃ group, in which R₃ is defined as in Formulas II and III and in which R′ is an unsubstituted or a substituted phenyl group.

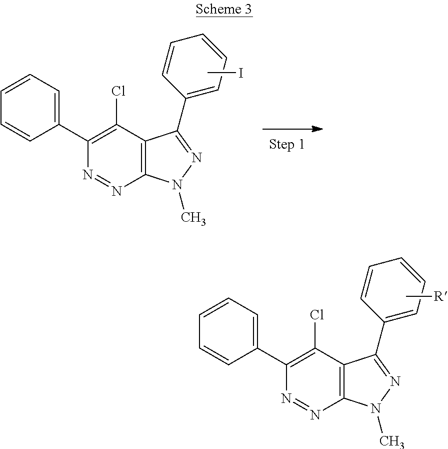

Scheme 3 generally describes the preparation of Pyrazolopyridazine compounds having a 1-N-methyl group and where R′ is a cyano group, an alkyne, an alkene or an aryl group. For example, 1-methyl-3-iodophenyl-4-chloro-5-phenyl-1H-pyrazolo[3,4-c]pyridazine is coupled with a suitable coupling partner, such as a cyanide salt, a terminal alkyne, an alkenyl halide, or an aryl halide, optionally in the presence of a suitable catalyst such as a palladium complex, optionally in the presence of a non-palladium transition metal salt such as a zinc or copper salt, optionally in the presence of an additive such as triphenylphosphine or an organic amine base, to provide a Pyrazolopyridazine compound having a 1-N-methyl group and where R′ is a cyano group, an alkyne, an alkene or an aryl group. The position of R′, i.e., ortho, meta or para, in the product is the same as the position of the iodo group in the starting material.

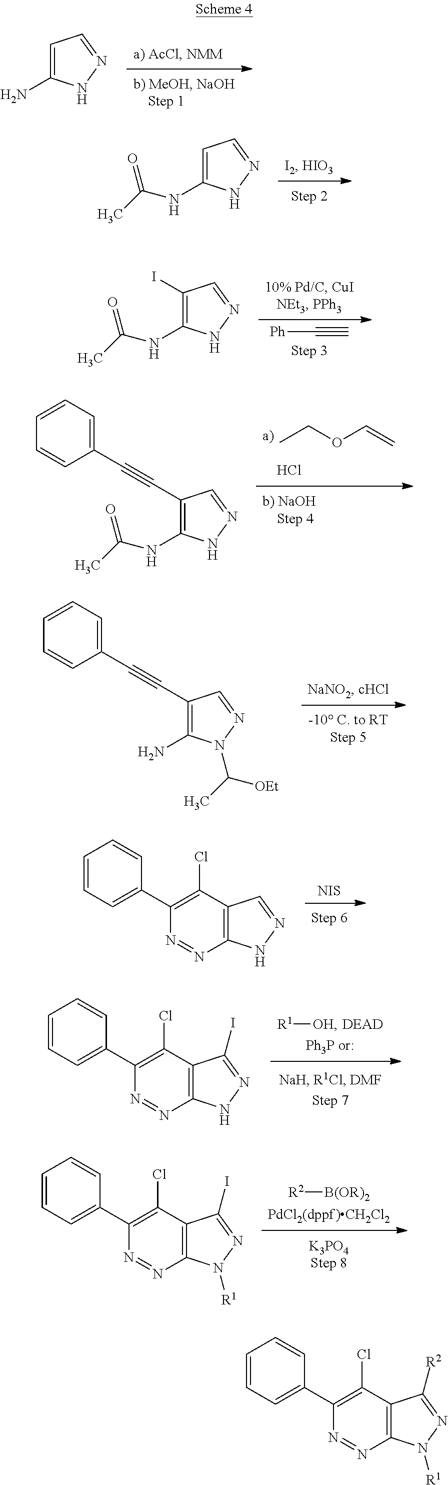

Scheme 4 generally describes the preparation of Pyrazolopyridazine compounds.

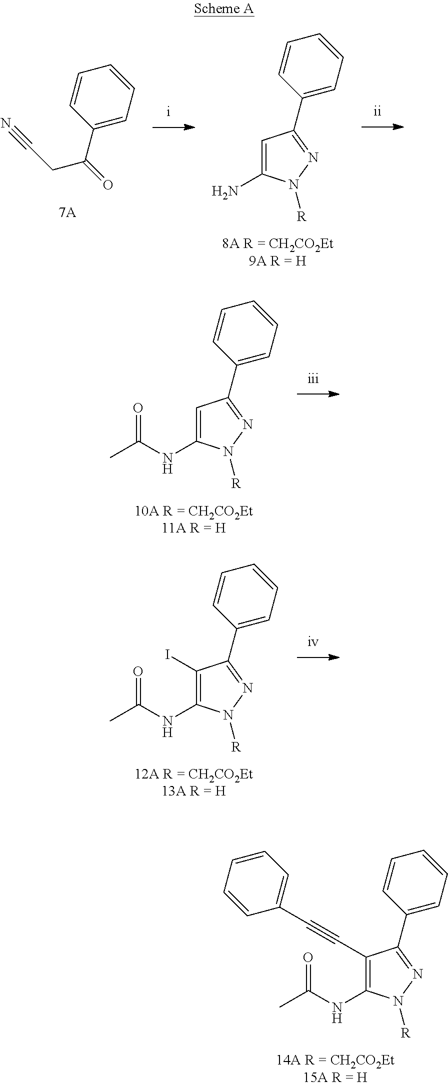

(i) ethyl hydrazinoacetate hydrochloride, EtOH, reflux, 2 h; (ii) Ac₂O, pyridine, 25° C., 16 h, or AcCl, N-methylmorpholine, CH₂Cl₂, 25° C., 3 h; (iii) I₂, HIO₃, EtOH, 50° C.; (iv) Phenyl acetylene, Pd(PPh₃)₂Cl₂, CuI, Et₃N, DMF, 90° C.

Scheme A generally describes the preparation of Ethyl 2-[5-acetamido-3-phenyl-4-(2-phenylethynyl)-1H-pyrazol-1-yl]acetate and N-[3-Phenyl-4-(2-phenylethynyl)-1H-pyrazol-5-yl]acetamide from benzoylacetonitrile.

### Compound 8A of Scheme A: Ethyl 2-(5-amino-3-(phenyl)-1H-pyrazol-1-yl)acetate

A mixture of benzoylacetonitrile (7A, 44 g, 304 mmol) and ethyl hydrazinoacetate hydrochloride (47 g, 304 mmol) in ethanol (400 mL) is heated to reflux for 2 h. The reaction mixture is concentrated in vacuo. The crude reaction mixture is partitioned between CH₂Cl₂ (400 mL) and saturated NaHCO₃ (aq). The aqueous phase is extracted with CH₂Cl₂ and the organic phases are combined, dried over MgSO₄, filtered and evaporated to give compound 8A as a solid (70 g, 95% yield). ¹H NMR (400 MHz, CDCl₃) δ (ppm) 7.73 (m, 2H), 7.38 (m, 2H), 7.26 (m, 1H), 5.96 (s, 1H), 4.86 (s, 2H), 4.25 (m, 2H), 3.7 (s, 2H), 1.28 (s, 3H).

### Compound 10A of Scheme A: Ethyl 2-(5-acetamido-3-phenyl-1H-pyrazol-1-yl)acetate

To a solution of ethyl 2-(5-amino-3-(phenyl)-1H-pyrazol-1-yl)acetate (8A, 41.7 g, 0.17 mol) in pyridine (200 mL) is added acetic anhydride (17.4 g, 0.17 mol) dropwise at 0° C. under an atmosphere of nitrogen. The reaction mixture is stirred at room temperature (RT) for 16 h. The reaction mixture is concentrated in vacuo. The residue is diluted with CH₂Cl₂ and water. The layers are separated and the organic layer is washed with water and brine, dried (MgSO₄) and concentrated in vacuo. CH₂Cl₂ is added to the residue and the solid is collected by filtration, yielding compound 10A as a solid (22 g, 45% yield). The mother liquors are concentrated in vacuo and washed with cold CH₂Cl₂ to give a second batch of compound 10A (15 g, 31% yield). ¹H NMR (400 MHz, DMSO-d₆) δ (ppm) 10.13 (s, 1H), 7.81 (d, J=7.6 Hz, 2H), 7.45 (t, J=7.6 Hz, 2H), 7.40-7.32 (m, 1H), 6.80 (s, 1H), 5.07 (s, 2H), 4.22 (q, J=7.1 Hz, 2H), 2.14 (s, 3H), 1.28 (t, J=7.1 Hz, 3H).

### Compound 11A of Scheme A: N-(3-Phenyl-1H-pyrazol-5-yl)acetamide

To a solution of 3-phenyl-1H-pyrazol-5-amine (9, 18.6 g, 0.117 mol) and N-methylmorpholine (30.8 mL, 0.281 mol) in CH₂Cl₂ (250 mL) is added acetyl chloride (20 mL, 0.281 mol) dropwise at 0° C. under an atmosphere of nitrogen. The reaction mixture is stirred at RT for 3 h. The reaction mixture is diluted with CH₂Cl₂ and water. The layers are separated and the organic layer is washed with water and brine, dried (phase separator cartridge) and concentrated in vacuo. Diethyl ether is added to the residue and the solid is collected by filtration, yielding compound 11A as a solid (25.1 g, 88% yield). ¹H NMR (400 MHz, DMSO-d₆) δ (ppm) 12.79 (s, 1H), 10.40 (s, 1H), 7.71 (d, J=7.5 Hz, 2H), 7.44 (dd, J=7.6, 7.6 Hz, 2H), 7.34 (dd, J=7.2, 7.2 Hz, 1H), 6.88 (s, 1H), 2.02 (s, 3H).

### Compound 12A of Scheme A: Ethyl 2-(5-acetamido-4-iodo-3-phenyl-1H-pyrazol-1-yl)acetate

A suspension of compound 10A (37 g, 129 mmol), iodic acid (5.6 g, 32 mmol) and iodine (19.7 g, 77 mmol) in ethanol (400 mL) is heated at 50° C. for 2 h and cooled to RT. The reaction mixture is concentrated in vacuo and the residue is eluted through a pad of silica gel with CH₂Cl₂/diethyl ether (1:0 to 97:3). The residue is partitioned between CH₂Cl₂ and 2 M Na₂S₂O₃ solution (aq). The layers are separated and the organic washed is dried (MgSO₄), and concentrated in vacuo to give a residue that is partially purified by chromatography (silica gel, CH₂Cl₂/isohexane 1:1 to 1:0, then CH₂Cl₂/diethyl ether 9:1 to 8:2), then triturated with diethyl ether yielding compound 12A as an off-white solid (43 g, 81% yield). ¹H NMR (400 MHz, CDCl₃) as a 3:1 mixture of rotamers δ (ppm) 7.81 (d, J=7.6 Hz, 2H), 7.45-7.35 (m, 3H), 7.15 (br s, 0.75H), 6.85 (br s, 0.25H), 4.97 (s, 2H), 4.25 (q, J=7.1 Hz, 2H), 2.24 (s, 2.25H), 2.04 (s, 0.75H), 1.30 (t, J=7.1 Hz, 3H).

### Compound 13A of Scheme A: N-(4-Iodo-3-phenyl-1H-pyrazol-5-yl)acetamide

A suspension of compound 11A (25.1 g, 0.103 mol), iodic acid (4.5 g, 0.026 mol) and iodine (15.7 g, 0.062 mol) in ethanol (250 mL) is heated at 50° C. for 3 h and cooled to RT. The reaction mixture i concentrated in vacuo and partitioned between CH₂Cl₂ and 2 M Na₂S₂O₃ solution (aq). The layers are separated and the organic washed with brine, dried (phase separator cartridge), and concentrated in vacuo to give a mixture of compound 13A and starting material 11A (2.2:1, 30.3 g). The mixture is put in reaction again using iodic acid (1.6 g, 9.6 mmol) and iodine (9.7 g, 38 mmol) in ethanol (250 mL) under the same conditions, to give compound 13A as a solid (31.9 g, 84% yield). ¹H NMR (400 MHz, CDCl₃) δ (ppm) 11.74-11.74 (m, 1H), 7.81 (d, J=7.2 Hz, 2H), 7.59 (s, 1H), 7.49-7.38 (m, 3H), 2.31 (s, 3H).

### Compound 14A of Scheme A: Ethyl 2-[5-acetamido-3-phenyl-4-(2-phenylethynyl)-1H-pyrazol-1-yl]acetate

Nitrogen is bubbled through a mixture of compound 12A (18.6 g, 45 mmol), phenyl acetylene (9.2 g, 90 mmol), copper iodide (860 mg, 4.5 mmol), triethylamine (200 mL) and DMF (75 mL) for 15 min. Bis(triphenylphosphine)palladium(II) dichloride (1.6 g, 2.25 mmol) is added and the reaction mixture is stirred at 90° C. under nitrogen for 4.5 h. The reaction mixture is cooled to RT, diluted with ethyl acetate and water. The organic phase is washed with water and brine, dried (MgSO₄), filtered and concentrated in vacuo. The residue is partially purified by column chromatography (silica gel, CH₂Cl₂, then isohexane/ethyl acetate 1:1 followed by CH₂Cl₂/ethyl acetate 9:1 to 8:2), then triturated with diethyl ether yielding compound 14A as a solid (13 g, 75% yield). ¹H NMR (400 MHz, DMSO-d₆) δ (ppm) 10.38 (s, 1H), 8.13-8.09 (m, 2H), 7.60-7.44 (m, 8H), 5.03 (s, 2H), 4.23 (q, J=7.1 Hz, 2H), 2.17 (s, 3H), 1.28 (t, J=7.1 Hz, 3H).

### Compound 15A of Scheme A: N-[3-Phenyl-4-(2-phenylethynyl)-1H-pyrazol-5-yl]acetamide

By a similar procedure to that described for the synthesis of compound 14A, compound 15A (12.5 g, 48% yield) is obtained from compound 13A (31.87 g, 86 mmol). ¹H NMR (400 MHz, CDCl₃) δ (ppm) 11.57-11.57 (m, 1H), 8.11 (d, J=7.4 Hz, 2H), 7.91 (s, 1H), 7.55-7.49 (m, 2H), 7.44 (dd, J=7.5, 7.5 Hz, 2H), 7.37 (dd, J=1.9, 5.0 Hz, 4H), 2.32 (s, 3H).

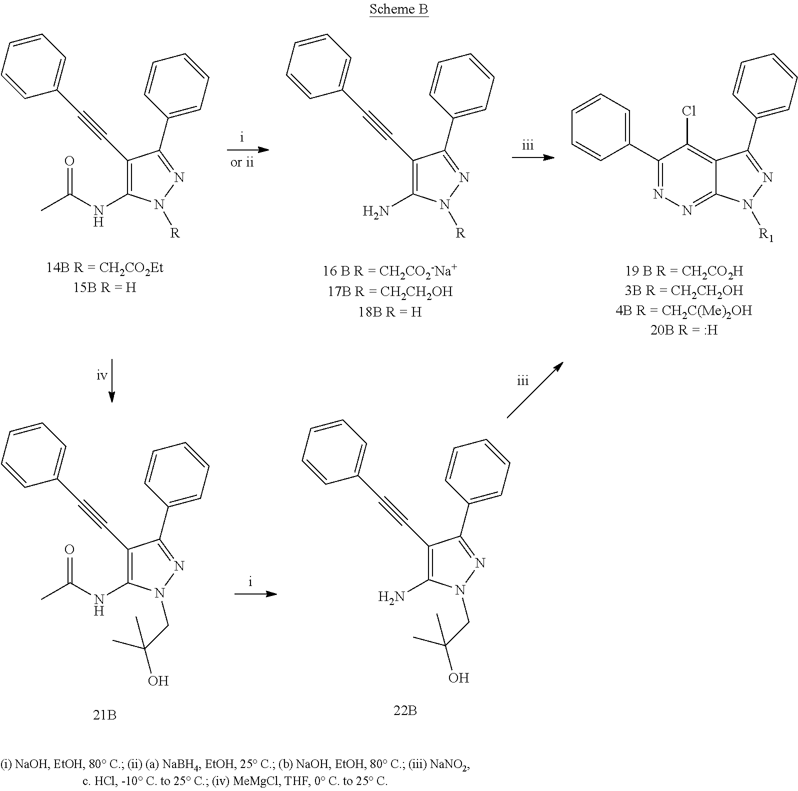

Scheme B generally describes the preparation of compound 3B and compound 4B.

### Compound 16B of Scheme B: Sodium 2-[5-amino-3-phenyl-4-(2-phenylethynyl)-1H-pyrazol-1-yl]acetate

A mixture of compound 14B (13 g, 34 mmol), ethanol (150 mL) and 25% NaOH solution (aq) (150 mL) is stirred and heated to 80° C. for 8 h and cooled to RT. Upon cooling, a precipitate is formed. The precipitate is filtered and washed with a cooled mixture of ethyl acetate/water (1:1). The solid is further triturated with diethyl ether, filtered and dried (MgSO₄), yielding compound 16B as a solid (9.8 g, 85% yield). ¹H NMR (400 MHz, DMSO-d₆) δ (ppm) 8.06 (d, J=7.8 Hz, 2H), 7.56 (d, J=7.6 Hz, 2H), 7.50-7.39 (m, 4H), 7.39-7.31 (m, 2H), 4.31 (s, 2H).

### Compound 17B of Scheme B: 2-[5-Amino-3-phenyl-4-(2-phenylethynyl-1H-)pyrazol-1-yl]ethan1-ol

To a suspension of compound 14B (22.4 g, 58 mmol) in ethanol (290 mL) is added sodium borohydride (11 g, 289 mmol) and the reaction mixture is stirred at RT for 16 h. The reaction mixture is partially concentrated to a final volume of 250 mL. A 25% NaOH solution (aq) (250 mL) is added and the reaction mixture is stirred at 80° C. for 4 h. The reaction mixture is cooled down to room temperature and phases are separated. The aqueous phase is extracted with ethyl acetate three times and the organic phases combined, dried (MgSO₄), filtered and concentrated in vacuo. The residue is triturated from diethyl ether (20 mL) and the product is filtered and dried in vacuo yielding compound 17B as an off-white solid (9.96 g, 57% yield). The mother liquor is concentrated in vacuo and purified by column chromatography (silica gel, gradient 0 to 100% ethyl acetate/isohexane) yielding a further crop (1.79 g, 10% yield). ¹H NMR (400 MHz, CDCl₃) δ (ppm) 7.78-7.74 (m, 4H), 7.55-7.48 (m, 6H), 4.98 (t, J=4.8 Hz, 2H), 4.29 (m, 2H), 3.03 (t, J=6.4 Hz, 1H).

### Compound 18B of Scheme B: 3-Phenyl-4-(2-phenylethynyl)-1H-pyrazol-5-amine

By a similar procedure to that described for the synthesis of compound 16B, compound 18B (5.4 g, 50% yield) is obtained from compound 15B (12.5 g, 41 mmol). ¹H NMR (400 MHz, CDCl₃) δ (ppm) 7.87 (d, J=7.2 Hz, 2H), 7.51-7.43 (m, 4H), 7.42-7.32 (m, 4H), 4.09 (s, 2H).

### Compound 19B of Scheme B: 2-(4-Chloro-3,5-diphenyl-1H-pyrazolo[3,4-c]pyridazin-1-yl)acetic acid

Sodium nitrite (1.86 g, 26.9 mmol) is added portionwise to cHCl (30 mL) at 0° C. and stirred for 15 min and then compound 16B (3 g, 8.85 mmol) is added as a solid to the reaction mixture, portionwise. The suspension is then stirred at RT for 16 h. The reaction mixture is diluted with CH₂Cl₂ and washed with water and brine. The organic layer is dried (MgSO₄) and concentrated in vacuo. The residue is purified by column chromatography (silica gel, diethyl ether/CH₂Cl₂ 1:9) yielding compound 19B as a solid (1.7 g, 53% yield). ¹H NMR (400 MHz, CDCl₃) δ (ppm) 7.80-7.72 (m, 4H), 7.56-7.47 (m, 6H), 5.64 (s, 2H), 2.10 (s, 1H).

### Compound 3B of Scheme B: 2-(4-Chloro-3,5-diphenyl-1H-pyrazolo[3,4-c]pyridazin-1-yl)ethan-1-ol

Sodium nitrite (4.58 g, 66.3 mmol) is added portionwise to cHCl (220 mL) at −10° C. and stirred for 10 min. Compound 17B (6.7 g, 22.1 mmol) is added as a solid. The reaction mixture is allowed to warm up, is sonicated for 5 min then stirred at RT for 2 h. The reaction mixture is diluted with CH₂Cl₂ and water and the aqueous phase is extracted with CH₂Cl₂. The organic phases are combined, dried (MgSO₄), filtered and concentrated in vacuo. The residue is partially purified by column chromatography (silica gel, gradient 0 to 100% ethyl acetate/isohexane). The resulting residue is triturated from diethyl ether then from ethyl acetate, yielding compound 3B as a solid (900 mg, 12% yield). ¹H NMR (400 MHz, CDCl₃) δ (ppm) 7.79-7.75 (m, 4H), 7.55-7.46 (m, 6H), 4.98 (m, 2H), 4.32-4.25 (m, 2H), 3.04 (t, J=6.4 Hz, 1H). ¹³C NMR (100 MHz, CDCl₃) δ (ppm) 153.09, 151.98, 143.65, 134.48, 130.11, 129.35, 129.33, 129.06, 128.29, 128.17, 127.39, 127.33, 113.70, 60.64, 50.63. LC-MS (analytical method 1: HPLC (Phenomenex Luna 5 μm C18, 100×4.6 mm) with gradient of 5-95% acetonitrile in water (with 0.1% formic acid in each mobile phase)) Rₜ 4.14 min; m/z 351 [M+H] 99.04% purity.

### Compound 21B of Scheme B: N-(1-(2-Hydroxy-2-methylpropyl)-3-phenyl-4-(phenylethynyl)-1H-pyrazol-5-yl)acetamide

To a solution of compound 14B (1.0 g, 2.58 mmol) in THF (26 mL) is added methyl magnesium chloride (3 M solution in THF, 3 mL, 9 mmol) at 0° C. The solution obtained is stirred at RT for 3.5 h then successively diluted with ethyl acetate and quenched by addition of 1 M HCl (aq). The aqueous phase is extracted with ethyl acetate, and the combined organic layers are dried (MgSO₄) and concentrated in vacuo. The resultant residue is purified using chromatography (silica gel, gradient 0 to 75% ethyl acetate/isohexane) yielding compound 21B as a solid (529 mg, 55% yield). ¹H NMR (400 MHz, CDCl₃) as a 1.5:1 mixture of compound 21B δ (ppm) 8.11 (dd, J=7.5, 12.3 Hz, 2H), 7.51-7.40 (m, 4H), 7.37-7.31 (m, 4H), 4.15-4.07 (m, 2H), 2.24-2.23 (m, 3H), 1.28 (s, 6H) and N-[2-acetonyl-5-phenyl-4-(2-phenylethynyl)pyrazol-3-yl]acetamide δ (ppm) 8.11 (dd, J=7.5, 12.3 Hz, 2H), 7.51-7.40 (m, 4H), 7.37-7.31 (m, 4H), 4.95 (s, 2H), 2.24-2.23 (m, 3H), 1.28 (s, 6H).

### Compound 22B of Scheme B: 1-(5-Amino-3-phenyl-4-(phenylethynyl)-1H-pyrazol-1-yl)-2-methylpropan-2-ol

Compound 22B (310 mg, yield 59%) is synthesized from compound 21B (597 mg, 1.6 mmol) following similar procedures outlined in the synthesis of compound 16B. ¹H NMR (400 MHz, CDCl₃) δ (ppm) 8.11-8.08 (m, 2H), 7.50-7.47 (m, 2H), 7.41 (dd, J=7.5, 7.5 Hz, 2H), 7.37-7.29 (m, 4H), 4.48 (s, 2H), 4.01 (s, 2H), 2.70 (s, 1H), 1.32-1.31 (m, 6H).

### Compound 4B of Scheme B: 1-(4-Chloro-3,5-diphenyl-1H-pyrazolo[3,4-c]pyridazin-1-yl)-2-methylpropan-2-ol

To cooled (cooling bath −15° C.) cHCl (9 mL) is added sodium nitrite in one portion (121 mg, 1.75 mmol) and the suspension is left to stir for 10 min after which compound 22B (290 mg, 0.88 mmol) is added. After 5 min, the cooling bath is removed and the reaction mixture is stirred at RT for 3 h. The reaction is cooled again (0° C.) and CH₂Cl₂ is added followed by water. The aqueous phase is extracted with CH₂Cl₂ and the organic phases are combined, dried (MgSO₄), filtered and concentrated in vacuo. Crude material is purified by column chromatography (silica gel, gradient 0 to 50% ethyl acetate/isohexane) yielding compound 4B as orange oil (56 mg). The material obtained is further purified by preparative HPLC, yielding compound 4B as a solid (34 mg, 10% yield). ¹H NMR (400 MHz, CDCl₃) δ (ppm) 7.81-7.75 (m, 4H), 7.55-7.50 (m, 6H), 4.85 (s, 2H), 3.50 (s, 1H), 1.36 (s, 6H). ¹³C NMR (100 MHz, CDCl₃) δ (ppm) 154.36, 152.91, 144.60, 135.42, 130.96, 130.29, 130.28, 129.93, 129.23, 129.07, 128.29, 128.24, 114.16, 71.52, 58.60, 27.26. LCMS (analytical method 1: HPLC (Phenomenex Luna 5 μm C18, 100×4.6 mm) with gradient of 5-95% acetonitrile in water (with 0.1% formic acid in each mobile phase)) Rₜ 4.49 min; m/z 379 [M+H] 99.71% purity.

### Compound 20B of Scheme B: 4-Chloro-3,5-diphenyl-1H-pyrazolo[3,4-c]pyridazine

Sodium nitrite (2.88 g, 42 mmol) is added portionwise to cHCl (314 mL) at −15° C. and stirred for 15 min. Compound 18B (5.4 g, 21 mmol) is added as a solid, followed by the addition of CH₂Cl₂ (10 mL). The reaction mixture is allowed to warm up and stirred at RT for 1 h. The reaction mixture is diluted with CH₂Cl₂ (44 mL) and NaCl (2.7 g) is added. The reaction mixture is heated to 50° C. for 1 d. The layers are separated and the organic layer is washed with water, dried (phase separator cartridge) and concentrated in vacuo. The residue is purified by column chromatography (silica gel, isohexane/ethyl acetate 4:1, then CH₂Cl₂/ethyl acetate 1:0 to 4:1) yielding compound 20B as a solid (3.0 g, 47% yield). ¹H NMR (400 MHz, DMSO-d₆) δ (ppm) 15.08 (s, 1H), 7.81-7.73 (m, 4H), 7.58-7.51 (m, 6H).

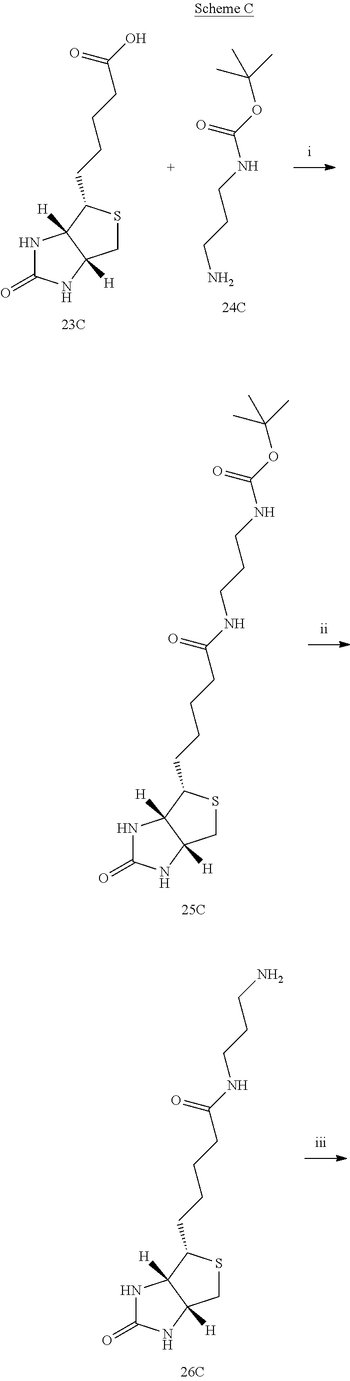 [SMILES: O=C(O)CCCC[C@@H]1SC[C@@H]2NC(=O)N[C@@H]21 ; CC(C)(C)OC(=O)NCCCN ; CC(C)(C)OC(=O)NCCCNC(=O)CCCC[C@@H]1SC[C@@H]2NC(=O)N[C@@H]21 ; NCCCNC(=O)CCCC[C@@H]1SC[C@@H]2NC(=O)N[C@@H]21]

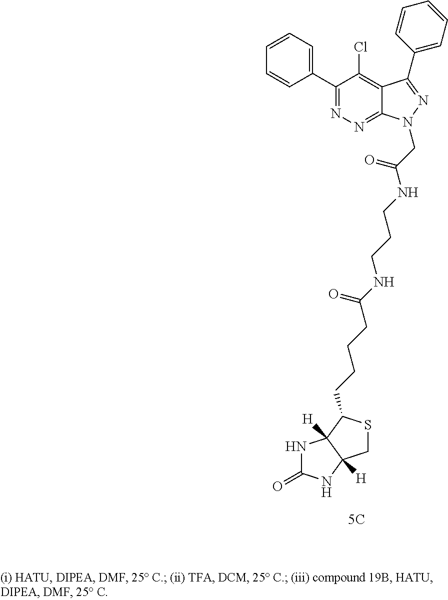 [SMILES: O=C(CCCC[C@@H]1SC[C@@H]2NC(=O)N[C@@H]21)NCCCNC(=O)Cn1nc(-c2ccccc2)c2c(Cl)c(-c3ccccc3)nnc21]

Scheme C generally describes the preparation of compound 5C.

### Compound 25C of Scheme C: tert-Butyl (3-(5-((3aS,4S,6aR)-2-oxohexahydro-1H-thieno[3,4-d]imidazol-4-yl)pentanamido)propyl)carbamate

To a solution of biotin (23C, 350 mg, 1.43 mmol) in DMF (7.2 mL) are added tert-butyl N-(3-aminopropyl)carbamate (24C, 250 mg, 1.43 mmol), DIPEA (0.375 mL, 2.15 mmol) and HATU (816 mg, 2.15 mmol). The reaction mixture is stirred at RT for 20 h, and then diluted with ethyl acetate and 4% LiCl aqueous solution. The aqueous phase is extracted with ethyl acetate twice, and the combined organic layers are dried (MgSO₄) and concentrated in vacuo. The resultant residue is purified using chromatography (silica gel, gradient 0 to 12% 7 M NH₃ in MeOH/CH₂Cl₂) yielding compound 25C as a solid (180 mg, 31% yield). ¹H NMR (400 MHz, DMSO-d₆) δ (ppm) 7.75 (t, J=5.2 Hz, 1H), 6.77 (s, 1H), 6.43 (s, 1H), 6.37 (s, 1H), 4.38-4.34 (m, 1H), 4.21-4.16 (m, 1H), 3.23 (d, J=5.3 Hz, 1H), 3.16 (dq, J=6.2, 4.3 Hz, 1H), 3.07 (dd, J=6.8, 12.9 Hz, 2H), 2.96 (dd, J=6.6, 13.0 Hz, 2H), 2.88 (dd, J=5.2, 12.4 Hz, 1H), 2.11 (t, J=7.5 Hz, 2H), 1.73-1.62 (m, 1H), 1.61-1.48 (m, 5H), 1.43 (s, 9H), 1.40-1.29 (m, 2H).

### Compound 26C of Scheme C: N-(3-Aminopropyl)-5-((3aS,4S,6aR)-2-oxohexahydro-1H-thieno[3,4-d]imidazol-4-yl)pentanamide

To a solution of compound 25C (166 mg, 0.415 mmol) in CH₂Cl₂ (1 mL) is added TFA (1 mL). The reaction mixture is stirred at RT for 2 h, then concentrated in vacuo. The resultant residue is dissolved in CH₂Cl₂ (2 mL) and Biotage MP-carbonate resin (550 mg, 1.66 mmol) is added. The reaction mixture is stirred at RT for 30 min. Beads are filtered off and washed with CH₂Cl₂/MeOH (1:1, 2 mL) and the filtrate is concentrated in vacuo to afford compound 26C as a colorless oil (124 mg, 100% yield), which is used as such in the next step.

### Compound 5C of Scheme C: N-(3-(2-(4-Chloro-3,5-diphenyl-1H-pyrazolo[3,4-c]pyridazin-1-yl)acetamido)propyl)-5-((3aS,4S,6aR)-2-oxohexahydro-1H-thieno[3,4-d]imidazol-4-yl)pentanamide

To a solution of compound 26C (124 mg, 0.415 mmol) in DMF (2 mL) are added compound 19B (151 mg, 0.415 mmol), DIPEA (0.11 mL, 0.62 mmol) and HATU (236 mg, 0.62 mmol). The reaction mixture is stirred at RT for 1.5 h and then diluted with CH₂Cl₂ and 4% LiCl aqueous solution. The aqueous phase is extracted with CH₂Cl₂ twice, and the combined organic layers are dried (phase separation) and concentrated in vacuo. The resultant residue is first purified by prep HPLC yielding 70 mg, which is further purified by silica gel chromatography, yielding the desired compound 5C as a solid (44 mg, 16% yield). ¹H NMR (400 MHz, DMSO-d₆) δ (ppm) 8.39 (dd, J=5.7, 5.7 Hz, 1H), 7.87-7.78 (m, 5H), 7.64-7.57 (m, 6H), 6.45 (s, 1H), 6.39 (s, 1H), 5.51 (s, 2H), 4.33 (dd, J=5.3, 7.6 Hz, 1H), 4.18-4.13 (m, 1H), 3.22-3.08 (m, 5H), 2.85 (dd, J=5.2, 12.5 Hz, 1H), 2.61 (d, J=12.4 Hz, 1H), 2.10 (dd, J=7.5, 7.5 Hz, 2H), 1.66-1.48 (m, 6H), 1.39-1.28 (m, 2H). ¹³C NMR (100 MHz, CDCl₃) δ (ppm) 172.51, 166.25, 163.16, 154.31, 152.75, 144.06, 135.95, 131.35, 130.58, 130.52, 129.58, 129.44, 129.42, 128.75, 128.70, 114.18, 61.47, 59.64, 55.85, 50.91, 37.21, 36.66, 35.66, 29.64, 28.66, 28.48, 25.75. LCMS (analytical method 1: HPLC (Phenomenex Luna 5 μm C18, 100×4.6 mm) with gradient of 5-95% acetonitrile in water (with 0.1% formic acid in each mobile phase)) Rₜ 3.52 min; m/z 647 [M+H] 98.38% purity.

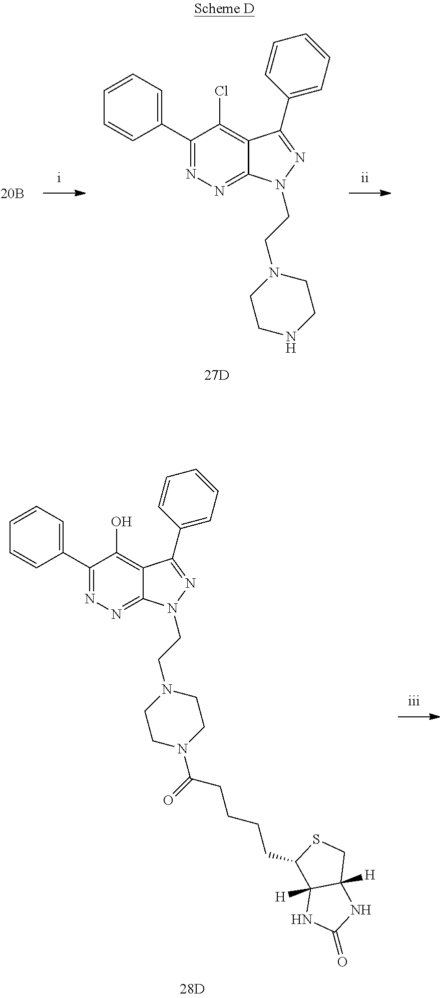 [SMILES: Clc1c(-c2ccccc2)nnc2c1c(-c1ccccc1)nn2CCN1CCNCC1 ; O=C1N[C@H]2[C@H](CS[C@H]2CCCCC(=O)N2CCN(CCn3nc(-c4ccccc4)c4c(O)c(-c5ccccc5)nnc43)CC2)N1]

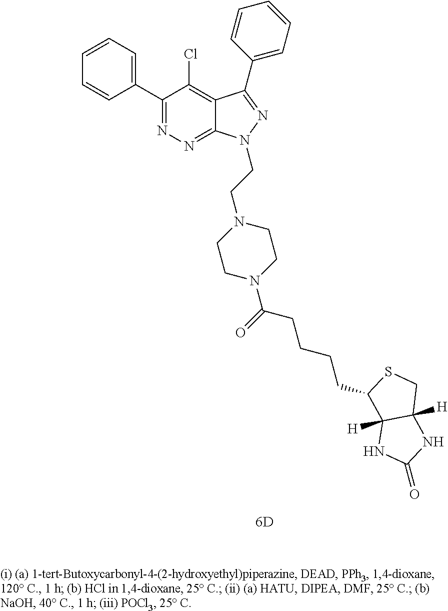 [SMILES: O=C1N[C@H]2[C@H](CS[C@H]2CCCCC(=O)N2CCN(CCn3nc(-c4ccccc4)c4c(Cl)c(-c5ccccc5)nnc43)CC2)N1]

Scheme D generally describes the preparation of compound 6D.

### Compound 27D of Scheme D: 4-Chloro-3,5-diphenyl-1-(2-(piperazin-1-yl)ethyl)-1H-pyrazolo[3,4-c]pyridazine

To a mixture of compound 20B (345 mg, 1.13 mmol), 1-tert-butoxycarbonyl-4-(2-hydroxyethyl)piperazine (520 mg, 2.26 mmol) and triphenylphosphine (888 mg, 2.26 mmol) in 1,4-dioxane (8.4 mL) is slowly added to diethyl azodicarboxylate (0.355 mL, 2.26 mmol) at RT. The reaction mixture is then heated using microwave irradiation to 120° C. for 1 h. The reaction mixture is cooled down to RT and 4 M HCl in 1,4-dioxane (4 mL) is added. The reaction mixture is stirred at RT for 4 h, diluted with CH₂Cl₂ (10 mL) and the solution is loaded onto a Biotage SCX-2 cartridge (20 g), eluted with methanol, then 7 M NH₃ in methanol. Fractions were concentrated in vacuo to give compound 27D in a 1:1 ratio with 2-hydroxyethyl)piperazine (950 mg, 100% yield) and is used as such in the next step.

### Compound 28D of Scheme D: (3aS,4S,6aR)-4-(5-(4-(2-(4-Hydroxy-3,5-diphenyl-1H-pyrazolo[3,4-c]pyridazin-1-yl)ethyl)piperazin-1-yl)-5-oxopentyl)tetrahydro-1H-thieno[3,4-d]imidazol-2(3H)-one

To a solution of compound 27D (950 mg crude, 1.13 mmol) in DMF (5.7 mL), DIPEA (0.59 mL, 3.39 mmol), biotin (678 mg, 2.78 mmol) and HATU (1.29 g, 3.39 mmol) are added. The reaction mixture is stirred at RT for 24 h and then diluted with DMSO (5 mL). NaOH (2 M, 5 mL) is added and the reaction mixture is heated to 40° C. for 1 h, then stirred at RT for 2 days. The reaction mixture is purified by preparative HPLC to yield compound 28D (100 mg, 14% yield). ¹H NMR

### Compound 6D of Scheme D: (3aS,4S,6aR)-4-(5-(4-(2-(4-Chloro-3,5-diphenyl-1H-pyrazolo[3,4-c]pyridazin-1-yl)ethyl)piperazin-1-yl)-5-oxopentyl)tetrahydro-1H-thieno[3,4-d]imidazol-2(3H)-one

A solution of compound 28D (97 mg, 0.155 mmol) in phosphorous oxychloride (6 mL) is stirred at RT for 2 days. The reaction mixture is diluted with CH₂Cl₂ and 2 M Na₂CO₃ solution. The aqueous phase is extracted twice with CH₂Cl₂. Combined organic layers are dried (MgSO₄) and concentrated in vacuo. The resultant residue is purified using chromatography (silica gel, gradient 0 to 12% 7 M NH₃ in MeOH/CH₂Cl₂) yielding compound 6D as a solid (42 mg, 42% yield). ¹H NMR (400 MHz, DMSO-d₆) δ (ppm) 7.86-7.78 (m, 4H), 7.66-7.56 (m, 6H), 6.47 (s, 1H), 6.39 (s, 1H), 5.01-4.94 (m, 2H), 4.38-4.32 (m, 1H), 4.20-4.15 (m, 1H), 3.17-3.10 (m, 1H), 3.08-3.01 (m, 2H), 2.87 (dd, J=12.4, 5.1 Hz, 1H), 2.63 (d, J=12.4 Hz, 1H), 2.58 (m, 1H), 2.57-2.54 (m, 2H), 2.51 (m, 2H), 2.36-2.28 (m, 2H), 1.71-1.61 (m, 1H), 1.56-1.45 (m, 3H), 1.38 (m, 2H), 1.27-1.17 (m, 1H). ¹³C NMR (100 MHz, CDCl₃) δ (ppm) 170.51, 162.70, 153.69, 151.95, 143.18, 135.52, 131.06, 130.15, 130.11, 129.03, 128.94, 128.84, 128.26, 128.20, 113.42, 61.04, 59.76, 59.19, 56.16, 55.48, 52.71, 52.28, 45.27, 32.05, 28.29, 28.11, 24.85. LCMS (analytical method 2: HPLC (Hichrom ACE 3 C18-AR mixed mode column 100×4.6 mm) with gradient of 2-100% acetonitrile in water (with 0.1% formic acid in each mobile phase)) Rₜ 9.77 min; m/z 645 [M+H] 93.27% purity.

Molecular Chaperones

Though there are numerous configurations for a particular protein to adopt as it folds, a protein that is ultimately biologically functional folds to a stable state, referred to as its “native state”, in which the protein's tendency to aggregate is at a minimum. The folding process of proteins is thermodynamically driven by the so-called “hydrophobic effect”, which is the tendency of a protein's hydrophobic amino acid residues to interact with one another and form a hydrophobic core, while the protein's hydrophilic amino acid residues remain at the protein's surface. Nascent or partially folded proteins are “sticky” because their hydrophobic amino acids are not completely buried in the protein's core. As a result, sticky proteins can clump together into intractable aggregates, especially in a cellular environment that is crowded with other protein molecules.

A quality-control system exists in a cell's cytoplasm and nucleus to ensure that protein folding occurs efficiently. This system includes molecular chaperones and the ubiquitin proteasome system (UPS). The UPS allows for tagging of proteins with ubiquitin to target them for degradation in a proteasome, a complex of protein molecules that degrade ubiquitinated polypeptides and recycle the ubiquitin tags.

Molecular chaperones are proteins that help other proteins fold efficiently by shielding the sticky hydrophobic surfaces of unfolded or misfolded proteins, thereby minimizing the proteins' tendency to aggregate. Molecular chaperones can be found in almost all organisms and are present in many cellular compartments. Some molecular chaperones are expressed constitutively and not induced by stress, others are expressed constitutively and induced by stress, and some are induced by stress. Molecular chaperones, such as the heat shock proteins (Hsps), are classified according to their molecular weight and include the small Hsps, Hsp40, Hsp60, Hsp70, Hsp90 and Hsp100 families (Table 2).

TABLE 2
Illustrative Families of Heat Shock Proteins

| | |
|---|---|
| Approximate | |
| molecular weight (kD) | Heat shock protein |

| | | |
|---|---|---|
| 10 | kD | HSP10 |
| 20-30 | kD | HSPB group, includes HSP27 |
| 40 | kD | HSP40 |
| 60 | kD | HSP60 |
| 70 | kD | HSPA group, includes HSP71, HSP70, |
| | | HSP72, Grp78 (BiP), Hsx70 in primates |
| 70 | kD | Ribosome-associated complex (RAC) |
| 90 | kD | HSPC group, includes HSP90, Grp94 |
| 100 | kD | HSPH group, includes HSP104, HSP110 |

In some embodiments, a Pyrazolopyridazine compound, non-Pyrazolopyridazine compound or a metabolite thereof binds to a molecular chaperone. In some embodiments, a Pyrazolopyridazine compound, non-Pyrazolopyridazine compound or a metabolite thereof covalently binds to a molecular chaperone.

In some embodiments, the binding of a Pyrazolopyridazine compound, non-Pyrazolopyridazine compound or a metabolite thereof to a molecular chaperone results in the treatment or prevention of a proteopathy in a subject in need thereof.

In another embodiment, the molecular chaperone is a member of the Hsp10 family, Hsp40 family, Hsp60 family, Hsp70 family, Hsp90 family, or Hsp100 family.

Proteopathies

The Pyrazolopyridazine compounds as well as the non-Pyrazolopyridazine compounds are useful for treating or preventing a proteopathy.

In some embodiments, the proteopathy is a neurodegenerative disease. Illustrative neurodegenerative diseases include, but are not limited to, Alzheimer's disease, progressive supranuclear palsy, dementia pugilistica, frontotemporal dementia and parkinsonism linked to chromosome 17 (FTDP-17), Lytico-Bodig disease, tangle-predominant dementia, ganglioma, gangliocytoma, meningioangiomatosis, subacute sclerosing panencephalitis, lead encephalopathy, tuberous sclerosis, Hallervorden-Spatz disease, lipofuscinosis, Pick's disease, corticobasal degeneration, argyrophilic grain disease, Huntington's disease, Parkinson's disease, dementia accompanied by Lewy bodies, multiple system atrophy, neuroaxonal dystrophies, dentatorubralpallidoluysian atrophy (DRPLA), spinal-bulbar muscular atrophy (SBMA), spinocerebellar ataxia 1 (SCA 1), SCA 2, SCA 3, SCA 6, SCA 7, SCA 17, prion disease, amyotrophic lateral sclerosis, frontotemporal lobar degeneration (FTLD) and familial encephalopathy accompanied by neuroserpin inclusion bodies (FENIB).

In some embodiments, the proteopathy is an amyloidosis. Illustrative amyloidoses include, but are not limited to, familial British dementia (ABri), familial Danish dementia (ADan), hereditary cerebral haemorrhage with amyloidosis-Icelandic (HCHWA-I), familial amyloidotic neuropathy (ATTR), AL (light chain) primary systemic amyloidosis, AH (heavy chain) amyloidosis, AA secondary amyloidosis, Aβ amyloidosis, aortic medial amyloidosis, LECT2 amyloidosis, AIAPP amyloidosis, apolipoprotein AI amyloidosis (AApoAI), apolipoprotein All amyloidosis (AApoAII), apolipoprotein AIV amyloidosis (AApoAIV), familial amyloidosis of the Finnish type (FAF), fibrinogen amyloidosis (AFib), lysozyme amyloidosis (ALys), dialysis amyloidosis (Aβ₂M), medullary thyroid carcinoma (ACal), cardiac atrial amyloidosis (AANF), pituitary prolactinoma (APro), hereditary lattice corneal dystrophy, cutaneous lichen amyloidosis (AKer), Mallory bodies, primary cutaneous amyloidosis, corneal lactoferrin amyloidosis, odontogenic (Pindborg) tumor amyloid or seminal vesicle amyloid.

In some embodiments, the proteopathy is a lysosomal storage disease. Illustrative lysosomal storage diseases include, but are not limited to, activator deficiency/GM2 gangliosidosis, alpha-mannosidosis, aspartylglucosaminuria, cholesteryl ester storage disease,chronic Hexosaminidase A Deficiency, cystinosis, Danon disease, Fabry disease, Farber disease, fucosidosis, galactosialidosis, Gaucher Disease Type I, Gaucher Disease Type II, Gaucher Disease Type III, GM1 gangliosidosis infantile, GM1 gangliosidosis late infantile/juvenile, GM1 gangliosidosis adult/chronic, I-Cell disease/Mucolipidosis II, Infantile Free Sialic Acid Storage Disease/ISSD, Juvenile Hexosaminidase A Deficiency, Krabbe disease infantile onset, Krabbe disease late onset, lysosomal acid lipase deficiency early onset, lysosomal acid lipase deficiency Late onset, Metachromatic Leukodystrophy, Pseudo-Hurler polydystrophy/Mucolipidosis IIIA, MPS I Hurler Syndrome, MPS I Scheie Syndrome, MPS I Hurler-Scheie Syndrome, MPS II Hunter syndrome, Sanfilippo syndrome Type A/MPS III A, Sanfilippo syndrome Type B/MPS III B, Sanfilippo syndrome Type C/MPS III C, Sanfilippo syndrome Type D/MPS III D, Morquio Type A/MPS IVA, Morquio Type B/MPS IVB, MPS IX Hyaluronidase Deficiency, MPS VI Maroteaux-Lamy, MPS VII Sly Syndrome, Mucolipidosis I/Sialidosis, Mucolipidosis IIIC, Mucolipidosis type IV, Multiple sulfatase deficiency, Niemann-Pick Disease Type A, Niemann-Pick Disease Type B, Niemann-Pick Disease Type C, CLN6 disease-atypical late infantile, CLN6 disease-late onset variant, CLN6 disease-early juvenile, Batten-Spielmeyer-Vogt/Juvenile NCL/CLN3 disease, Finnish Variant Late Infantile CLN5, Jansky-Bielschowsky disease/Late infantile CLN2/TPP1 Disease, Kufs/Adult-onset NCL/CLN4 disease, Northern Epilepsy/variant late infantile CLN8, Santavuori-Haltia/Infantile CLN1/PPT disease, Beta-mannosidosis, Pompe disease/glycogen storage disease type II, Pycnodysostosis, Sandhoff disease/Adult Onset/GM2 Gangliosidosis, Sandhoff disease/GM2 gangliosidosis—Infantile, Sandhoff disease/GM2 gangliosidosis—Juvenile, Schindler disease, Salla disease/Sialic Acid Storage Disease, Tay-Sachs/GM2 gangliosidosis or Wolman disease.

In some embodiments, the lysosomal storage disease is a mucopolysaccharidosis disorder. Illustrative mucopolysaccharidosis disorders include, but are not limited to Pseudo-Hurler polydystrophy/Mucolipidosis IIIA, MPS I Hurler Syndrome, MPS I Scheie Syndrome, MPS I Hurler-Scheie Syndrome, MPS II Hunter syndrome, Sanfilippo syndrome Type A/MPS III A, Sanfilippo syndrome Type B/MPS III B, Sanfilippo syndrome Type C/MPS III C, Sanfilippo syndrome Type D/MPS III D, Morquio Type A/MPS IVA, Morquio Type B/MPS IVB, MPS IX Hyaluronidase Deficiency, MPS VI Maroteaux-Lamy, MPS VII Sly Syndrome, Mucolipidosis I/Sialidosis, Mucolipidosis IIIC and Mucolipidosis type IV.

In other embodiments, the lysosomal storage disease is Pompe disease/glycogen storage disease type II.

In some embodiments, the proteopathy is a retinal degenerative disease. Non-limiting examples of retinal degenerative diseases include: retinitis pigmentosa, Leber's congenital Amaurosis, Syndromic retinal degenerations, age-related macular degeneration including wet and dry age-related macular degeneration, and Usher Syndrome. In some embodiments, the Usher Syndrome is a subtype of Usher Syndrome. In some embodiments, the subtype is Usher I. In some embodiments, the subtype is Usher II. In some embodiments, the subtype is Usher III.

In a further embodiment of the invention, a compound of the invention can be administered to a subject in need thereof for the treatment of hearing loss associated with Usher Syndrome. In some embodiments, the Usher Syndrome is a subtype of Usher Syndrome. In some embodiments, the subtype is Usher I. In some embodiments, the subtype is Usher II. In some embodiments, the subtype is Usher III.

Additional illustrative proteopathies include, but are not limited to, those disclosed in Table 3.

TABLE 3
Illustrative Proteopathies
Proteopathy

Alzheimer's disease
progressive supranuclear palsy
dementia pugilistica
frontotemporal dementia and parkinsonism linked to chromosome
17 (FTDP-17)
Lytico-Bodig disease
tangle-predominant dementia
ganglioma
gangliocytoma
meningioangiomatosis
subacute sclerosing panencephalitis
lead encephalopathy
tuberous sclerosis
Hallervorden-Spatz disease
Lipofuscinosis
Pick's disease
corticobasal degeneration
argyrophilic grain disease
Parkinson's disease
dementia with Lewy bodies
multiple system atrophy
neuroaxonal dystrophies
Huntington's disease
dentatorubralpallidoluysian atrophy (DRPLA)
spinal-bulbar muscular atrophy (SBMA)
spinocerebellar ataxia 1 (SCA 1)
SCA 2
SCA 3/Machado-Joseph disease
SCA 6
SCA 7
SCA 17
Prion diseases
Amyotrophic lateral sclerosis (ALS)
Frontotemporal lobar degeneration (FTLD) (Ubi+, Tau−)
FTLD-FUS
α1-antitrypsin deficiency
thrombosis
emphysema
types 1 and 2 hereditary angioedema (HAE)
familial encephalopathy
Familial British dementia, ABri (cerebral amyloid angiopathy)
Familial Danish dementia, Adan (cerebral amyloid angiopathy)
Hereditary cerebral hemorrhage with amyloidosis (Icelandic) (HCHWA-I)
Familial amyloidotic neuropathy, Senile systemic amyloidosis (ATTR)
AL (light chain) amyloidosis (primary systemic amyloidosis)
AH (heavy chain) amyloidosis
AA (secondary) amyloidosis
Aβ amyloidosis
AIAPP amyloidosis
Aortic medial amyloidosis
ApoAI amyloidosis (AApoAI)
ApoAII amyloidosis (AApoAII)
ApoAIV amyloidosis (AApoAIV)
Familial amyloidosis of the Finnish type (FAF)
Lysozyme amyloidosis (ALys)
Fibrinogen amyloidosis (AFib)
Dialysis amyloidosis (Aβ₂M)
Medullary thyroid carcinoma (ACal)
Cardiac atrial amyloidosis (AANF)
Pituitary prolactinoma (APro)
LECT2 amyloidosis
Hereditary lattice corneal dystrophy
Cutaneous lichen amyloidosis (AKer)
Mallory bodies
Primary cutaneous amyloidosis
Corneal lactoferrin amyloidosis
Odontogenic (Pindborg) tumor amyloid
Seminal vesicle amyloid
nephrogenic diabetes insipidus (NDI)
Iatrogenic (insulin)
Inclusion body myositis/myopathy
Cancer
Cataracts
Retinitis pigmentosa
Pulmonary alveolar proteinosis
Cystic Fibrosis
Sickle cell disease
Critical illness myopathy (CIM)
Wilson disease
Marfan syndrome
Fragile X syndrome
Fragile XE syndrome
Myotonic dystrophy
Retinal ganglion cell degeneration in glaucoma
Cerebral β-amyloid angiopathy
CADASIL (Cerebral Autosomal Dominant Arteriopathy with Sub-
cortical Infarcts and Leukoencephalopathy)
Alexander disease
Seipinopathies
Activator Deficiency/GM2 Gangliosidosis
Alpha-mannosidosis
Aspartylglucosaminuria
Cholesteryl ester storage disease
Chronic Hexosaminidase A Deficiency
Cystinosis
Danon disease
Fabry disease
Farber disease
Fucosidosis
Galactosialidosis
Gaucher Disease Type I
Gaucher Disease Type II
Gaucher Disease Type III
GM1 gangliosidosis Infantile
GM1 gangliosidosis Late infantile/Juvenile
GM1 gangliosidosis Adult/Chronic
I-Cell disease/Mucolipidosis II
Infantile Free Sialic Acid Storage Disease/ISSD
Juvenile Hexosaminidase A Deficiency
Krabbe disease Infantile Onset
Krabbe disease Late Onset
Lysosomal acid lipase deficiency Early onset
Lysosomal acid lipase deficiency Late onset
Metachromatic Leukodystrophy
Pseudo-Hurler polydystrophy/Mucolipidosis IIIA
MPS I Hurler Syndrome
MPS I Scheie Syndrome
MPS I Hurler-Scheie Syndrome
MPS II Hunter syndrome
Sanfilippo syndrome Type A/MPS III A
Sanfilippo syndrome Type B/MPS III B
Sanfilippo syndrome Type C/MPS III C
Sanfilippo syndrome Type D/MPS III D
Morquio Type A/MPS IVA
Morquio Type B/MPS IVB
MPS IX Hyaluronidase Deficiency
MPS VI Maroteaux-Lamy
MPS VII Sly Syndrome
Mucolipidosis I/Sialidosis
Mucolipidosis IIIC
Mucolipidosis type IV
Multiple sulfatase deficiency
Niemann-Pick Disease Type A
Niemann-Pick Disease Type B
Niemann-Pick Disease Type C
CLN6 disease - Atypical Late Infantile
CLN6 disease - Late Onset variant
CLN6 disease - Early Juvenile
Batten-Spielmeyer-Vogt/Juvenile NCL/CLN3 disease
Finnish Variant Late Infantile CLN5
Jansky-Bielschowsky disease/Late infantile CLN2/TPP1 Disease
Kufs/Adult-onset NCL/CLN4 disease
Northern Epilepsy/variant late infantile CLN8
Santavuori-Haltia/Infantile CLN1/PPT disease
Beta-mannosidosis
Pompe disease/Glycogen storage disease type II
Pycnodysostosis
Sandhoff disease/Adult Onset/GM2 Gangliosidosis
Sandhoff disease/GM2 gangliosidosis - Infantile
Sandhoff disease/GM2 gangliosidosis - Juvenile
Schindler disease
Salla disease/Sialic Acid Storage Disease
Tay-Sachs/GM2 gangliosidosis
Wolman disease

### Cystic Fibrosis

Cystic Fibrosis (CF), also known as mucoviscidosis, is an autosomal recessive disease that affects mostly the lungs but can also impact other organs, such as the pancreas, liver, kidneys and intestines. The name “cystic fibrosis” refers to the characteristic fibrosis and cysts that form within the pancreas. The disease is characterized by difficulty in breathing and by coughing up sputum due to frequent lung infections.

The most common mutation found in about 70% of CF sufferers worldwide is a deletion of a phenylalanine residue at amino acid position 508 in the 1480 amino acid cystic fibrosis transmembrane conductance regulator (CFTR) protein. This mutation causes misfolding of the protein and its degradation by the cell. Other mutations of the CFTR protein can result in truncated versions of the CFTR protein because their production is ended prematurely. Still other mutations produce mutant CFTR proteins that do not use energy normally; that do not allow chloride, iodide or thiocyanate to cross the membrane normally; or that degrade at a faster rate than normal. CFTR protein mutations can also lead to fewer copies of the CFTR protein being produced. A list of CFTR protein mutation classes and illustrative mutations of each class are disclosed in Table 4.

TABLE 4
CF mutation classes and illustrative mutations

| CF mutation classes | Illustrative mutations |
|---|---|
| I | W1282X, R553X, G542X |
| II | ΔF508, N1303K |
| III | G551D, G551S, G1349D |
| IV | R117H, R334W, R347P |
| V | 2789 + 5G > A, A455E |
| VI | 120Δ23, N287Y, 4326ΔITC, 4279insA |

Other CFTR protein mutations include G178R, S549N, S549R, G1244E, S1251N and S1255P.

Accordingly, the present invention further provides methods for treating or preventing cystic fibrosis, comprising administering to a subject in need thereof an effective amount of a Pyrazolopyridazine compound or a non-Pyrazolopyridazine compound. In some embodiments, the subject has a CFTR protein mutation. In one embodiment, the subject has a class I CF mutation. In another embodiment, the subject has a class II CF mutation. In another embodiment, the subject has a class III CF mutation. In another embodiment, the subject has a class IV CF mutation. In another embodiment, the subject has a class V CF mutation. In another embodiment, the subject has a class VI CF mutation.

In one embodiment, the mutation is W1282X, R553X or G542X. In another embodiment, the mutation is ΔF508 or N1303K. In another embodiment, the mutation is G551D, G551S or G1349D. In another embodiment, the mutation is R117H, R334W or R347P. In another embodiment, the mutation is 2789+5G>A or A455E. In another embodiment, the mutation is 120Δ23, N287Y, 4326ΔITC or 4279insA.

In one embodiment, the subject has one or more of the following mutations: W1282X, R553X, G542X, ΔF508, N1303K, G551D, G551S, G1349D, R117H, R334W, R347P, 2789+5G>A, A455E, 120Δ23, N287Y, 4326ΔITC, 4279insA, G178R, S549N, S549R, G1244E, S1251N and S1255P.

### Retinitis Pigmentosa

Retinitis Pigmentosa (RP) is an inherited, degenerative eye disease that causes severe vision impairment due to progressive degeneration of the rod photoreceptor cells in the retina. The progressive rod degeneration can be followed by abnormalities in the adjacent retinal pigment epithelium (RPE) and the deterioration of cone photoreceptor cells.

There are multiple genes that, when mutated, can cause RP. Inheritance patterns of RP have been identified as autosomal dominant, autosomal recessive, X-linked, and maternally (mitochondrially) acquired. X-linked RP can be either recessive, affecting primarily only males, or dominant, affecting both males and females. Some digenic (controlled by two genes) and mitochondrial forms of RP are also known.

A mutation of the gene for rhodopsin, a pigment that plays an essential role in the visual transduction cascade enabling vision in low-light conditions, has been identified. The mutation substitutes a proline at amino acid position 23 to a histidine. The rhodopsin gene is a principal protein of photoreceptor outer segments and consists of opsin, a light-sensitive membrane-bound G protein-coupled receptor and a reversibly covalently bound cofactor, retinal. Rhodopsin gene mutations most frequently follow autosomal dominant inheritance patterns.

Up to 150 RP-associated opsin gene mutations have been reported since the P23H mutation in the intradiscal domain of the protein was first reported. These mutations are found throughout the opsin gene and are distributed along the three domains of the protein (the intradiscal, transmembrane, and cytoplasmic domains). Mutations in the opsin gene are most commonly missense mutations and cause misfolding of the rhodopsin protein. The mutation of amino acid 23 in the opsin gene, in which proline is replaced with histidine, accounts for the largest percentage of rhodopsin mutations in the United States. Other mutations associated with RP include T58R, P347L, P347S, as well as deletion of Ile 255. The rare P23A mutation causes autosomal dominant RP.

A list of genes mutated in a subject having RP and the type of RP is presented in Table 5.

TABLE 5
Illustrative Retinitis Pigmentosa types and genes affected

| Gene | Type |
|---|---|
| RP1 | Retinitis pigmentosa-1 |
| RP2 | Retinitis pigmentosa-2 |
| RPGR | Retinitis pigmentosa-3, Retinitis pigmentosa- |
| | 15, X-linked |
| RHO | Retinitis pigmentosa-4 |
| | Retinitis pigmentosa-6, X-linked |
| ROM1 | Retinitis pigmentosa-7 |
| RP9 | Retinitis pigmentosa-9 |
| IMPDH1 | Retinitis pigmentosa-10 |
| PRPF31 | Retinitis pigmentosa-11 |
| CRB1 | Retinitis pigmentosa-12, autosomal recessive |
| PRPF8 | Retinitis pigmentosa-13 |
| TULP1 | Retinitis pigmentosa-14 |
| CA4 | Retinitis pigmentosa-17 |
| PRPF3 | Retinitis pigmentosa-18 |
| ABCA4 | Retinitis pigmentosa-19 |
| RPE65 | Retinitis pigmentosa-20 |
| | Retinitis pigmentosa-22 |
| OFD1 | Retinitis pigmentosa-23 |
| | Retinitis pigmentosa-24, X-linked |
| EYS | Retinitis pigmentosa-25 |
| CERKL | Retinitis pigmentosa-26 |
| NRL | Retinitis pigmentosa-27 |
| FAM161A | Retinitis pigmentosa-28 |
| FSCN2 | Retinitis pigmentosa-30 |
| TOPORS | Retinitis pigmentosa-31 |
| | Retinitis pigmentosa-32 |
| SNRNP200 | Retinitis pigmentosa-33 |
| | Retinitis pigmentosa-34, X-linked |
| SEMA4A | Retinitis pigmentosa-35 |
| PRCD | Retinitis pigmentosa-36 |
| NR2E3 | Retinitis pigmentosa-37 |
| MERTK | Retinitis pigmentosa-38 |
| USH2A | Retinitis pigmentosa-39 |
| PDE6B | Retinitis pigmentosa-40 |
| PROM1 | Retinitis pigmentosa-41 |
| KLHL7 | Retinitis pigmentosa-42 |
| PDE6A | Retinitis pigmentosa-43 |
| RGR | Retinitis pigmentosa-44 |
| CNGB1 | Retinitis pigmentosa-45 |
| IDH3B | Retinitis pigmentosa-46 |
| SAG | Retinitis pigmentosa-47 |
| GUCA1B | Retinitis pigmentosa-48 |
| CNGA1 | Retinitis pigmentosa-49 |
| BEST1 | Retinitis pigmentosa-50 |
| TTC8 | Retinitis pigmentosa-51 |
| RDH12 | Retinitis pigmentosa-53 |
| C2orf71 | Retinitis pigmentosa-54 |
| ARL6 | Retinitis pigmentosa-55 |
| IMPG2 | Retinitis pigmentosa-56 |
| PDE6G | Retinitis pigmentosa-57 |
| ZNF513 | Retinitis pigmentosa-58 |
| DHDDS | Retinitis pigmentosa-59 |
| PRPF6 | Retinitis pigmentosa-60 |
| CLRN1 | Retinitis pigmentosa-61 |
| MAK | Retinitis pigmentosa-62 |
| | Retinitis pigmentosa-63, autosomal dominant |
| C8ORF37 | Retinitis pigmentosa-64 |
| RBP3 | Retinitis pigmentosa-66 |
| NEK2 | Retinitis pigmentosa-67 |
| SLC7A14 | Retinitis pigmentosa-68 |
| KIZ | Retinitis pigmentosa-69 |
| PRPF4 | Retinitis pigmentosa-70 |
| PRPH2 | Retinitis pigmentosa, autosomal dominant |
| LRAT | Retinitis pigmentosa, juvenile |
| SPATA7 | Retinitis pigmentosa, juvenile, autosomal |
| | recessive |
| AIPL1 | Retinitis pigmentosa, juvenile |
| CRX | Retinitis pigmentosa, autosomal dominant |
| MT-TS2 | Retinitis pigmentosa, mitochondrial |
| RLBP1 | Retinitis pigmentosa, Bothnia retinal |
| | dystrophy, Newfoundland rod-cone dystrophy, |
| | Pigmentary retinal dystrophy |
| WDR19 | Retinitis pigmentosa, autosomal recessive |

Accordingly, the present invention further provides methods for treating or preventing retinitis pigmentosa, comprising administering to a subject in need thereof an effective amount of a Pyrazolopyridazine compound or non-Pyrazolopyridazine compound. In some embodiments, the subject has an RP gene mutation.

In one embodiment, the retinitis pigmentosa is autosomal dominant, autosomal recessive, X-linked or mitochondrially acquired.

In one embodiment, the retinitis pigmentosa (RP) is RP-1, RP-2, RP-3, RP-4, RP-6, RP-7, RP-9, RP-10, RP-11, RP-12, RP-13, RP-14, RP-15, RP-17, RP-18, RP-19, RP-20, RP-22,RP-23, RP-24, RP-25, RP-26, RP-27, RP-28, RP-30, RP-31, RP-32, RP-33, RP-34, RP-35, RP-36, RP-37, RP-38, RP-39, RP-40, RP-41, RP-42, RP-43, RP-44, RP-45, RP-46, RP-47, RP-48, RP-49, RP-50, RP-51, RP-53, RP-54, RP-55, RP-56, RP-57, RP-58, RP-59, RP-60, RP-61, RP-62, RP-63, RP-64, RP-66, RP-67, RP-68, RP-69 or RP-70.

In one embodiment, the subject has a mutation in one or more of the following genes: RP1, RP2, RPGR, RHO, ROM1, RP9, IMPDH1, PRPF31, CRB1, PRPF8, TULP1, CA4, PRPF3, ABCA4, RPE65, OFD1, EYS, CERKL, NRL, FAM161A, FSCN2, TOPORS, SNRNP200, SEMA4A, PRCD, NR2E3, MERTK, USH2A, PDE6B, PROM1, KLHL7, PDE6A, RGR, CNGB1, IDH3B, SAG, GUCA1B, CNGA1, BEST1, TTC8, RDH12, C2orf71, ARL6, IMPG2, PDE6G, ZNF513, DHDDS, PRPF6, CLRN1, MAK, C8ORF37, RBP3, NEK2, SLC7A14, KIZ, PRPF4, PRPH2, LRAT, SPATA7, AIPL1, CRX, MT-TS2, RLBP1 and WDR19.

### Therapeutic or Prophylactic Uses

The Pyrazolopyridazine compounds and non-Pyrazolopyridazine compounds can be administered to a subject as a component of a composition that comprises a pharmaceutically acceptable carrier or vehicle. Non-limiting examples of suitable pharmaceutical carriers or vehicles include starch, glucose, lactose, sucrose, gelatin, malt, rice, flour, chalk, silica gel, magnesium carbonate, magnesium stearate, sodium stearate, glycerol monostearate, talc, sodium chloride, dried skim milk, glycerol, propylene, glycol, water, ethanol, buffered water, and phosphate buffered saline. These compositions can be administered as, for example, drops, solutions, suspensions, tablets, pills, capsules, powders, and sustained-release formulations. In some embodiments, the compositions comprise, for example, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water syrup, methyl cellulose, methyl and propylhydroxybenzoates, talc, magnesium stearate, and mineral oil. The compositions can additionally comprise lubricating agents, wetting agents, emulsifying and suspending agents, preserving agents, sweetening agents or flavoring agents.

The compositions can comprise an effective amount of a Pyrazolopyridazine compound or non-Pyrazolopyridazine compound. The compositions can be formulated in a unit dosage form that comprises an effective amount of a Pyrazolopyridazine compound or non-Pyrazolopyridazine compound. In some embodiments, the compositions comprise, for example, from about 1 ng to about 1,000 mg of a Pyrazolopyridazine compound or non-Pyrazolopyridazine compound. In some embodiments, the compositions comprise from about 100 mg to about 1,000 mg of a Pyrazolopyridazine compound or non-Pyrazolopyridazine compound. In some embodiments, the compositions comprise from about 100 mg to about 500 mg of a Pyrazolopyridazine compound or non-Pyrazolopyridazine compound. In some embodiments, the compositions comprise from about 200 mg to about 300 mg of a Pyrazolopyridazine compound or non-Pyrazolopyridazine compound.

The dosage of a Pyrazolopyridazine compound or non-Pyrazolopyridazine compound can vary depending on the symptoms, age, and body weight of the subject, the nature and severity of the proteopathy, the route of administration, and the form of the composition. The compositions described herein can be administered in a single dose or in divided doses. In some embodiments, the dosage of a Pyrazolopyridazine compound or non-Pyrazolopyridazine compound ranges from about 0.01 ng to about 10 g per kg body mass of the subject, from about 1 ng to about 0.1 g per kg, or from about 100 ng to about 10 mg per kg.

Administration can be, for example, topical, intraaural, intraocular, parenteral, intravenous, intra-arterial, subcutaneous, intramuscular, intracranial, intraorbital, intraventricular, intracapsular, intraspinal, intracisternal, intraperitoneal, intranasal, aerosol, suppository, or oral. Formulations for oral use include tablets containing a Pyrazolopyridazine compound or non-Pyrazolopyridazine compound in a mixture with non-toxic pharmaceutically acceptable excipients. These excipients can be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc). Formulations for ocular use can be in the form of eyedrops.

A Pyrazolopyridazine compound, non-Pyrazolopyridazine compound or compositions thereof can be provided in lyophilized form for reconstituting, for instance, in isotonic, aqueous, or saline buffers for parental, subcutaneous, intradermal, intramuscular, or intravenous administration. A composition can also be in the form of a liquid preparation useful for oral, intraaural, nasal, or sublingual administration, such as a suspension, syrup or elixir. A composition can also be in a form suitable for oral administration, such as a capsule, tablet, pill, and chewable solid formulation. A composition can also be prepared as a cream for dermal administration as a liquid, a viscous liquid, a paste, or a powder. A composition can also be prepared as a powder for pulmonary administration with or without an aerosolizing component.

The compositions can be in oral, intraaural, intranasal, sublingual, intraduodenal, subcutaneous, buccal, intracolonic, rectal, vaginal, mucosal, pulmonary, transdermal, intradermal, parenteral, intravenous, intramuscular and ocular dosage forms as well as being able to traverse the blood-brain barrier.

The compositions can be administered by various means known in the art. For example, the compositions can be administered orally, and can be formulated as tablets, capsules, granules, powders or syrups. Alternatively, compositions can be administered parenterally as injections (for example, intravenous, intramuscular or subcutaneous), drop infusion preparations or suppositories. For ophthalmic application compositions can be formulated as eye drops or eye ointments. Aural compositions can be formulated as ear drops, ointments, creams, liquids, gels, or salves for application to the ear, either internally or superficially. These formulations can be prepared by conventional means, and the compositions can be mixed with any conventional additive, such as an excipient, a binder, a disintegrating agent, a lubricant, a solubilizing agent, a suspension aid, an emulsifying agent, or a coating agent.

Compositions can include wetting agents, emulsifiers, and lubricants, coloring agents, release agents, coating agents, sweetening, flavoring and perfuming agents, preservatives and antioxidants.

Compositions can be suitable, for example, for oral, intraaural, intraocular, nasal, topical (including buccal and sublingual), rectal, vaginal, aerosol and/or parenteral administration. The compositions can be provided in a unit dosage form, and can be prepared by any methods known in the art.

Formulations suitable for oral administration may be in the form of capsules, cachets, pills, tablets, lozenges, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia. Compositions can also be administered as a bolus, electuary, or paste.

Additional examples of pharmaceutically acceptable carriers or vehicles include: (1) fillers or extenders, such as starches, lactose, sucrose, glucose, mannitol, and/or silicic acid; (2) binders, such as carboxymethyl cellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as acetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof (10) coloring agents; and (11) buffering agents. Similar compositions can be employed as fillers in soft- or hard-filled gelatin capsules.

Liquid dosage forms for oral administration include pharmaceutically acceptable emulsions, microemulsions, gels, solutions, suspensions, syrups and elixirs. The liquid dosage form can contain inert diluents commonly used in the art, for example, water or other solvents, solubilizing agents and emulsifiers, such as ethyl alcohol, isopropyl alcohol, diethyl carbonate, ethyl acetate, benzyl alcohol, benzyl benzoate, propylene glycol, 1,3-butylene glycol, oils such as, cottonseed, groundnut, corn, germ, olive, castor and sesame oils, glycerol, tetrahydrofuryl alcohol, polyethylene glycols, fatty acid esters of sorbitan, and mixtures thereof.

Suspension dosage forms can contain suspending, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The dosage forms for transdermal administration of a subject composition include drops, powders, sprays, ointments, pastes, creams, lotions, gels, solutions, and patches. The ointments, pastes, creams, and gels can contain excipients, such as animal and vegetable fats, oils, waxes, paraffin, starch, tragacanth, cellulose derivatives, polyethylene glycols, silicones, bentonite, silicic acid, talc and zinc oxide, or mixtures thereof.

Powders and sprays can contain excipients such as lactose, talc, silicic acid, aluminum hydroxide, calcium silicates, polyamide powder, or mixtures thereof. Sprays may additionally contain customary propellants, such as chlorofluorohydrocarbons and volatile unsubstituted hydrocarbons, such as butane and propane.

Compositions can be administered by aerosol of solid particles. A non-aqueous (e.g., fluorocarbon propellant) suspension could be used. Sonic nebulizers can be used because they minimize exposure to shear, which might cause degradation.

An aqueous aerosol can be made by formulating an aqueous solution or suspension of a Pyrazolopyridazine compound or non-Pyrazolopyridazine compound with any conventional pharmaceutically acceptable carriers or vehicles such non-ionic surfactants (Tweens, Pluronics, or polyethylene glycol); proteins such as serum albumin; sorbitan esters; fatty acids; lecithin; amino acids; buffers; salts; sugars; or sugar alcohols.

Compositions suitable for parenteral administration comprise a Pyrazolopyridazine compound or non-Pyrazolopyridazine compound and one or more pharmaceutically acceptable sterile isotonic aqueous or non-aqueous solutions, dispersions, suspensions, or emulsions, or sterile powders which can be reconstituted into sterile injectable solutions or dispersions just prior to use, which can contain antioxidants, buffers, bacteriostats, or solutes, which render the formulation isotonic with the blood of the subject, and suspending or thickening agents.

Having described the invention with reference to certain embodiments, other embodiments will become apparent to one skilled in the art from consideration of the specification and claims. It will be apparent to those skilled in the art that many modifications, both to materials and methods, may be practiced without departing from the scope of the invention.

### INCORPORATION BY REFERENCE

Each reference disclosed in this application is incorporated by reference herein in its entirety.

1. A method for treating or preventing a proteopathy, comprising administering to a subject in need thereof an effective amount of a compound of Formula II: 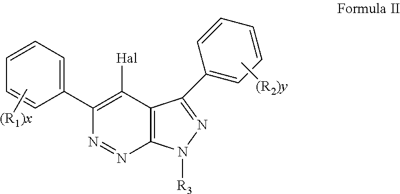
or a pharmaceutically acceptable salt thereof,
wherein Hal is —Cl, —F, —I, or —Br;
x is an integer ranging from 0 to 5;
each R₁ is independently —Cl, —F, —I, —Br, —C₁-C₃ alkyl, —O—C₁-C₃ alkyl, —CN, —CF₃, —C(O)NH(CH₃), or —C≡CCH₂OH;
y is an integer ranging from 0 to 5;
each R₂ is independently —Cl, —F, —Br, —C₁-C₃ alkyl, —O—C₁-C₃ alkyl, —CN, —CF₃, —C(O)NH(CH₃), or —C≡CCH₂OH;
R₃ is —H, —C₁-C₆ alkyl, —(C₁-C₆ alkylene)-OH, —(C₁-C₆ alkylene)-phenyl, —(C₁-C₆ alkylene)-O—(C₁-C₆ alkyl), —C₂-C₆ alkenyl, —(C₁-C₆ alkylene)-C(O)R₄, —(C₁-C₆ alkylene)-R₅, 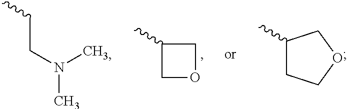
R₄ is —OH, —O—(C₁-C₆ alkyl), —NH₂, —NH(C₁-C₆ alkyl), —NH((C₁-C₆ alkylene)-OH), —NH((C₁-C₆ alkylene)N(C₁-C₆ alkyl)₂), —N(C₁-C₆ alkyl)((C₁-C₆ alkylene)-CN), —N(C₁-C₆ alkyl)((C₁-C₆ alkylene)N(C₁-C₆ alkyl)₂), —NH(C₁-C₆ alkylene)-O—(C₁-C₆ alkyl), 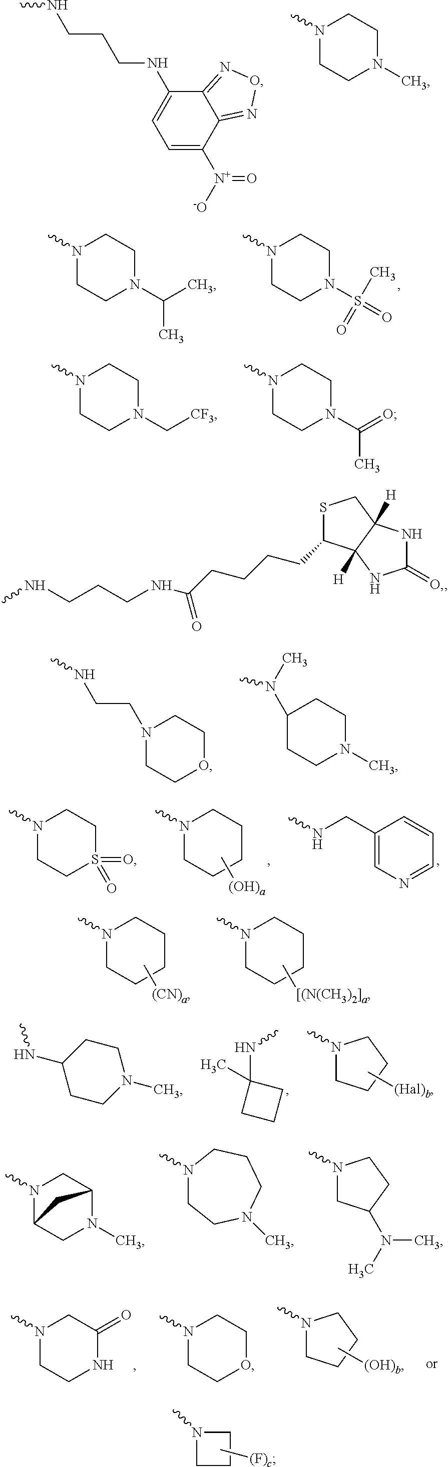
a is an integer ranging from 0 to 10;
b is an integer ranging from 0 to 8;
c is an integer ranging from 0 to 6; and
R₅ is

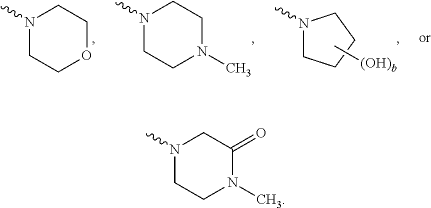

2. The method of claim 1, wherein the compound has the structure: 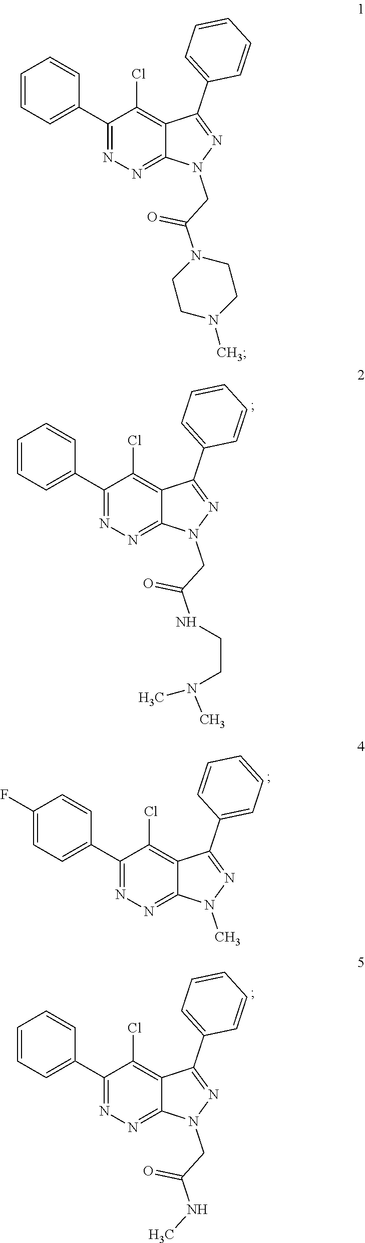 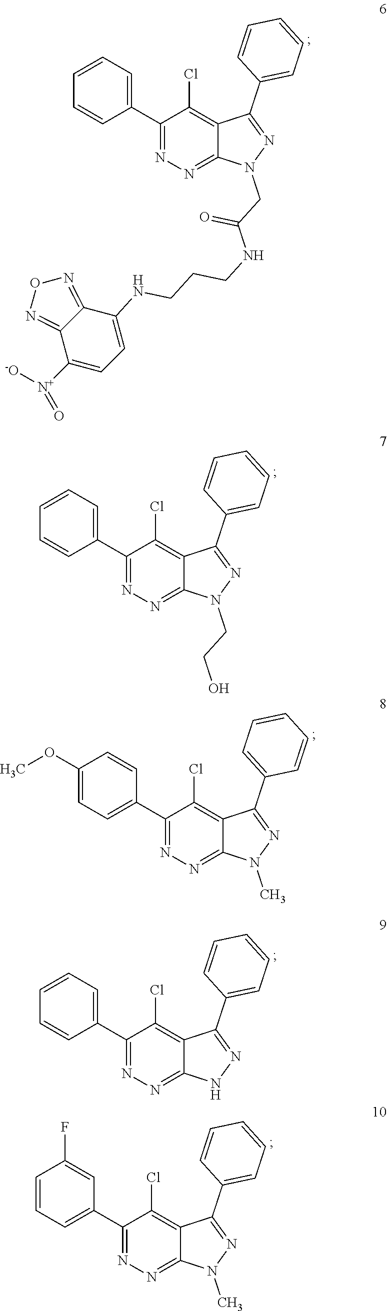 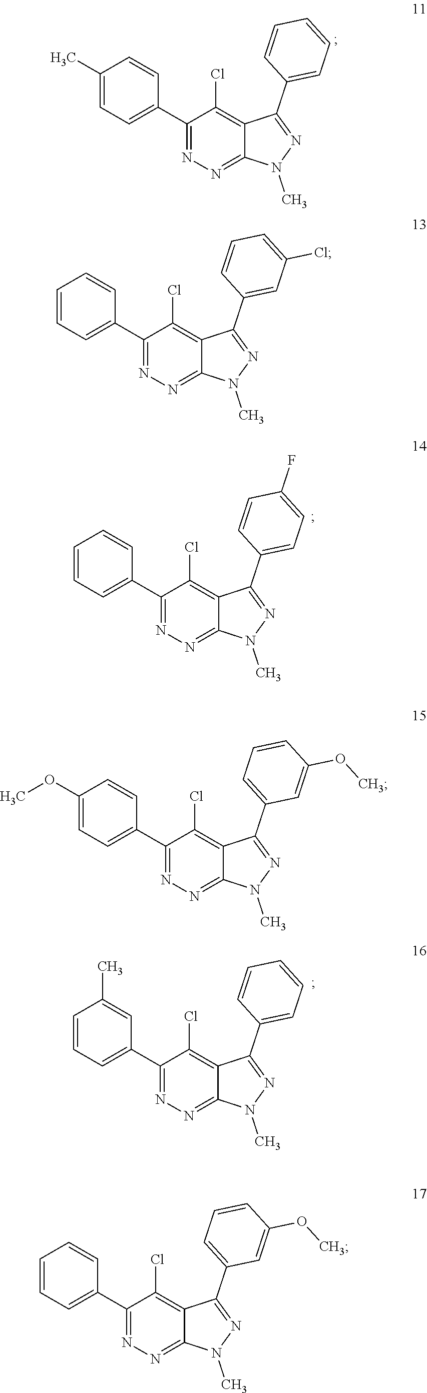 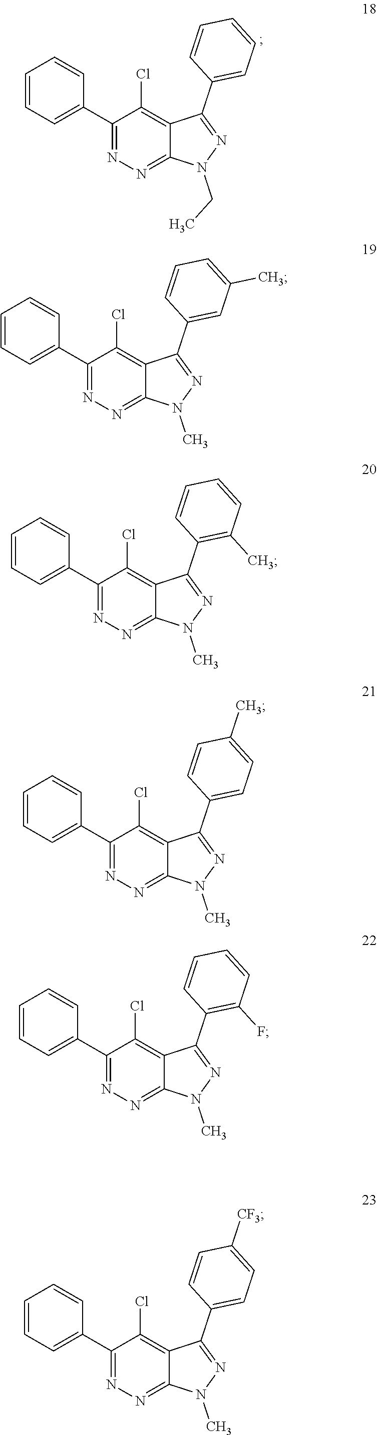 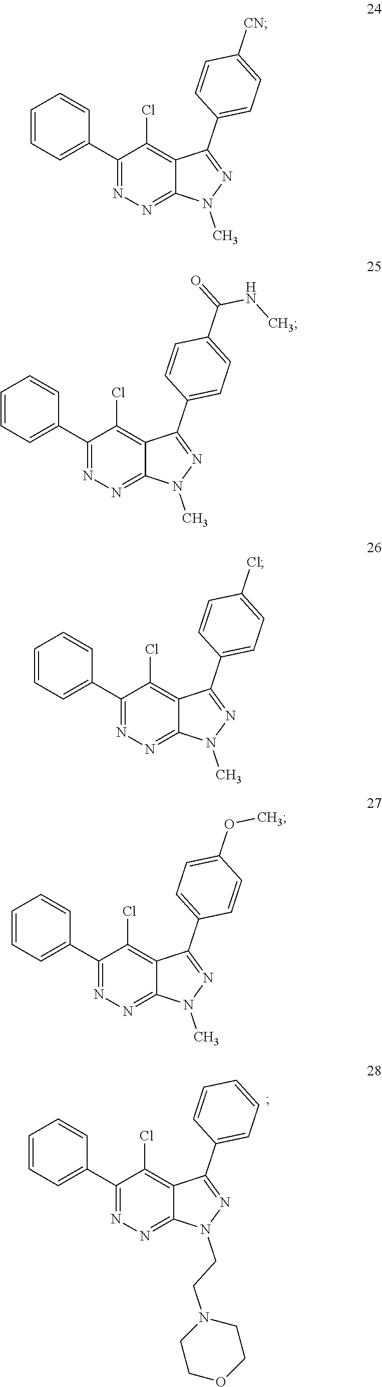 [SMILES: Cn1nc(-c2ccc(C#N)cc2)c2c(Cl)c(-c3ccccc3)nnc21 ; CNC(=O)c1ccc(-c2nn(C)c3nnc(-c4ccccc4)c(Cl)c23)cc1 ; Cn1nc(-c2ccc(Cl)cc2)c2c(Cl)c(-c3ccccc3)nnc21 ; COc1ccc(-c2nn(C)c3nnc(-c4ccccc4)c(Cl)c23)cc1 ; Clc1c(-c2ccccc2)nnc2c1c(-c1ccccc1)nn2CCN1CCOCC1] 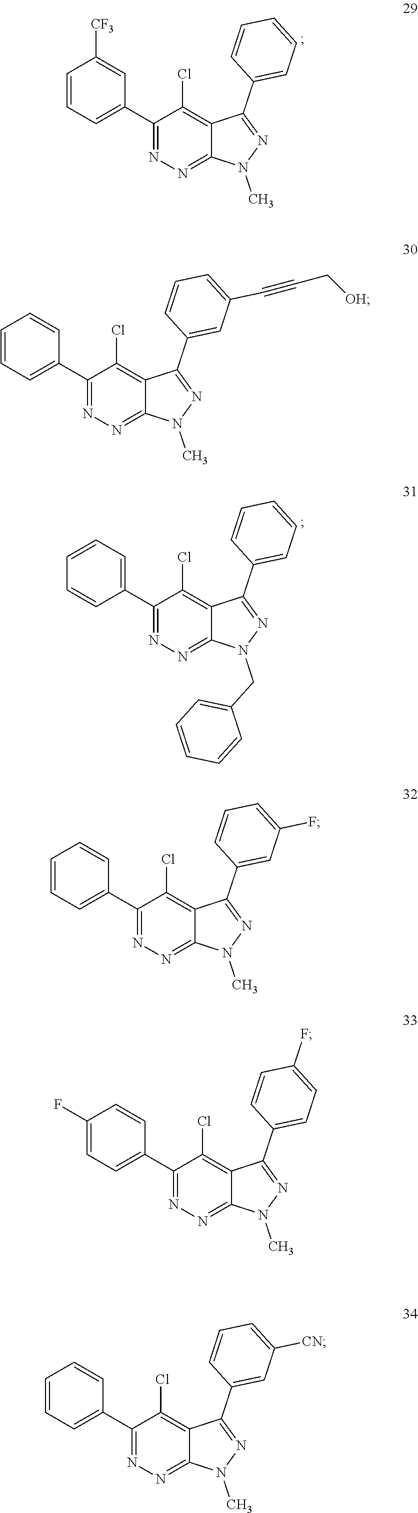 [SMILES: Cn1nc(-c2ccccc2)c2c(Cl)c(-c3cccc(C(F)(F)F)c3)nnc21 ; Cn1nc(-c2cccc(C#CCO)c2)c2c(Cl)c(-c3ccccc3)nnc21 ; Clc1c(-c2ccccc2)nnc2c1c(-c1ccccc1)nn2Cc1ccccc1 ; Cn1nc(-c2cccc(F)c2)c2c(Cl)c(-c3ccccc3)nnc21 ; Cn1nc(-c2ccc(F)cc2)c2c(Cl)c(-c3ccc(F)cc3)nnc21 ; Cn1nc(-c2cccc(C#N)c2)c2c(Cl)c(-c3ccccc3)nnc21] 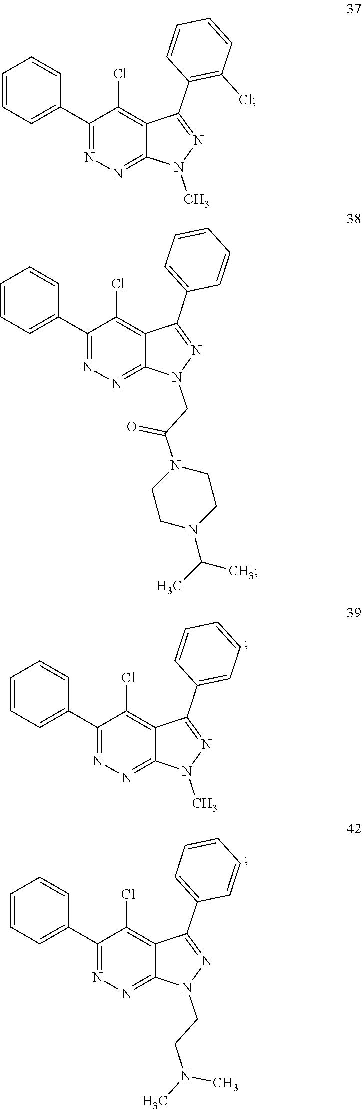 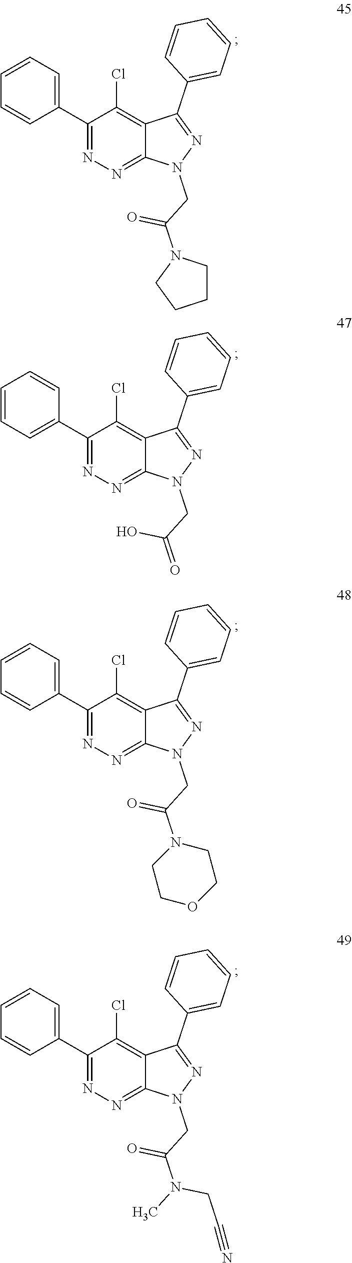 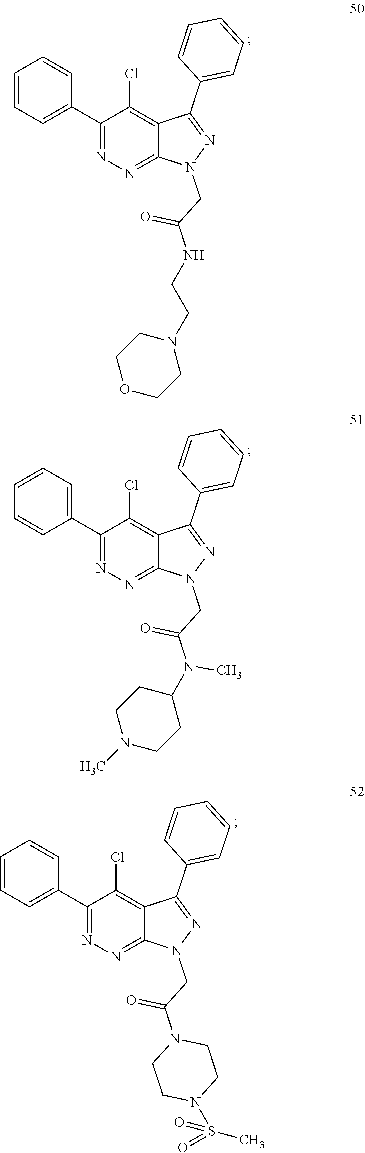 [SMILES: O=C(Cn1nc(-c2ccccc2)c2c(Cl)c(-c3ccccc3)nnc21)NCCN1CCOCC1 ; CN1CCC(N(C)C(=O)Cn2nc(-c3ccccc3)c3c(Cl)c(-c4ccccc4)nnc32)CC1 ; CS(=O)(=O)N1CCN(C(=O)Cn2nc(-c3ccccc3)c3c(Cl)c(-c4ccccc4)nnc32)CC1] 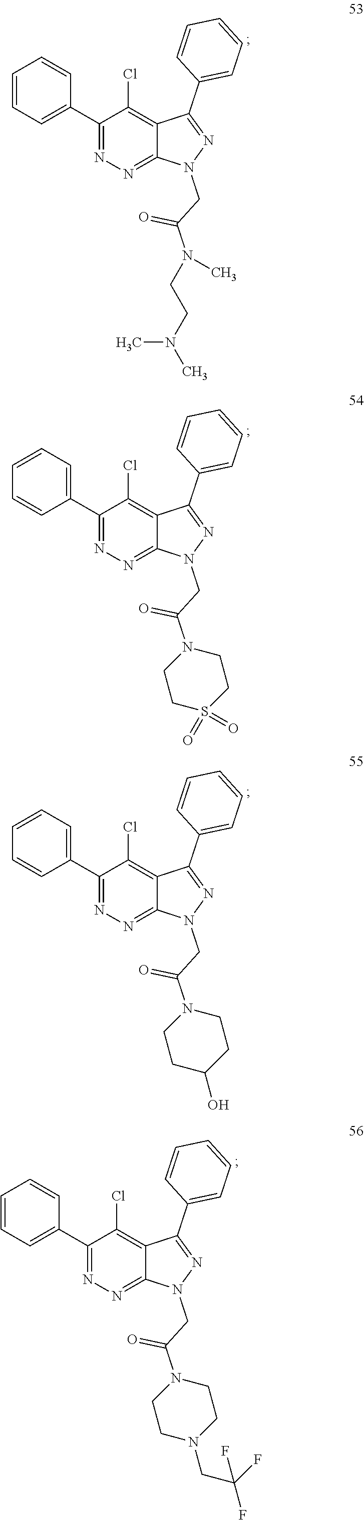 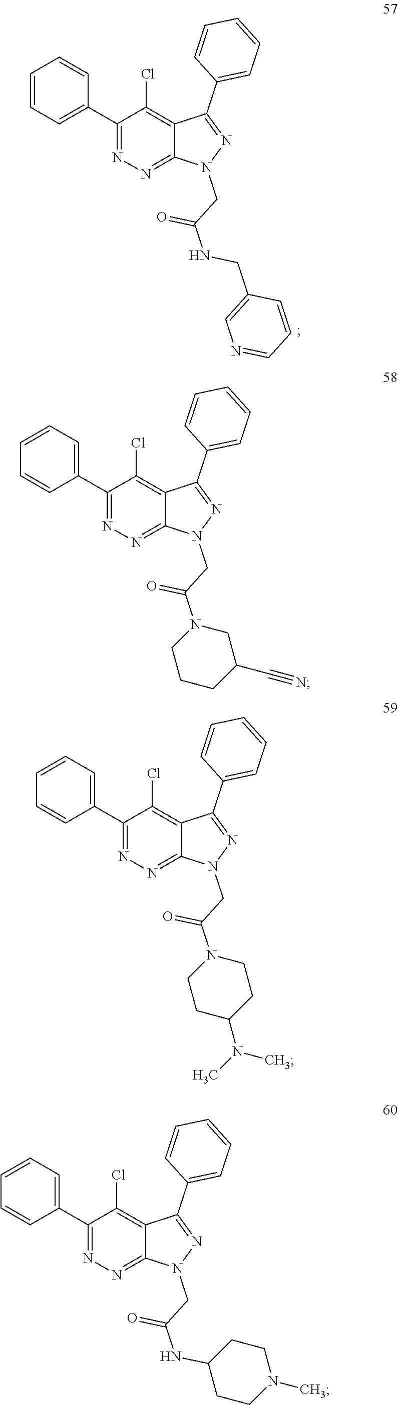 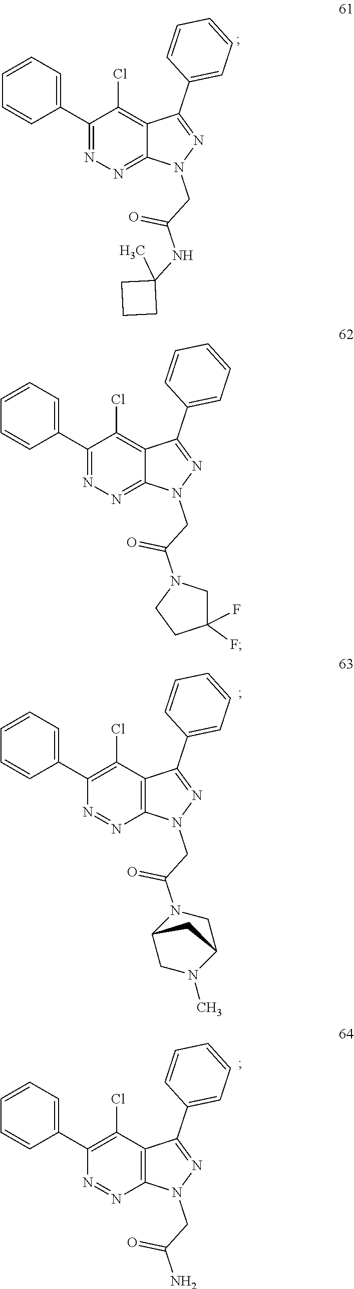 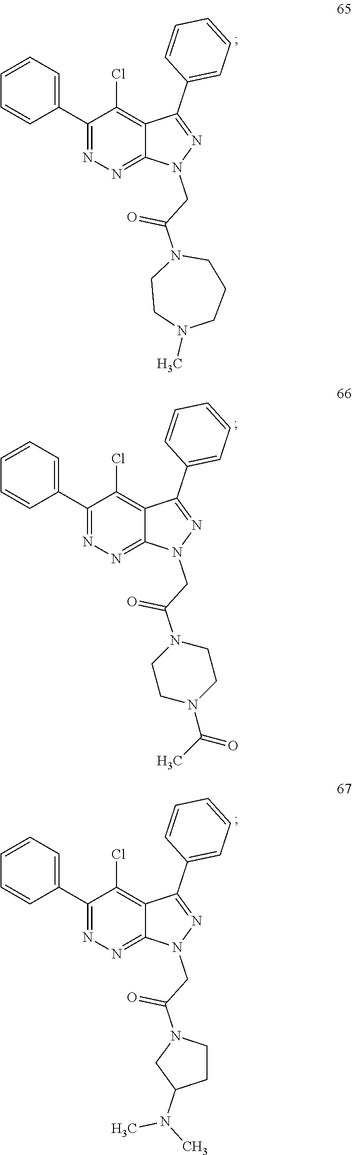 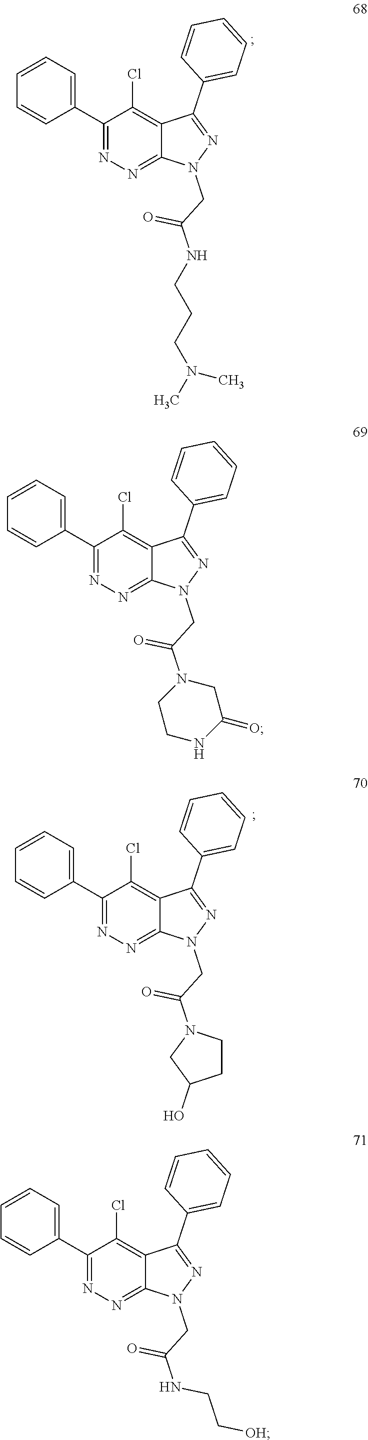 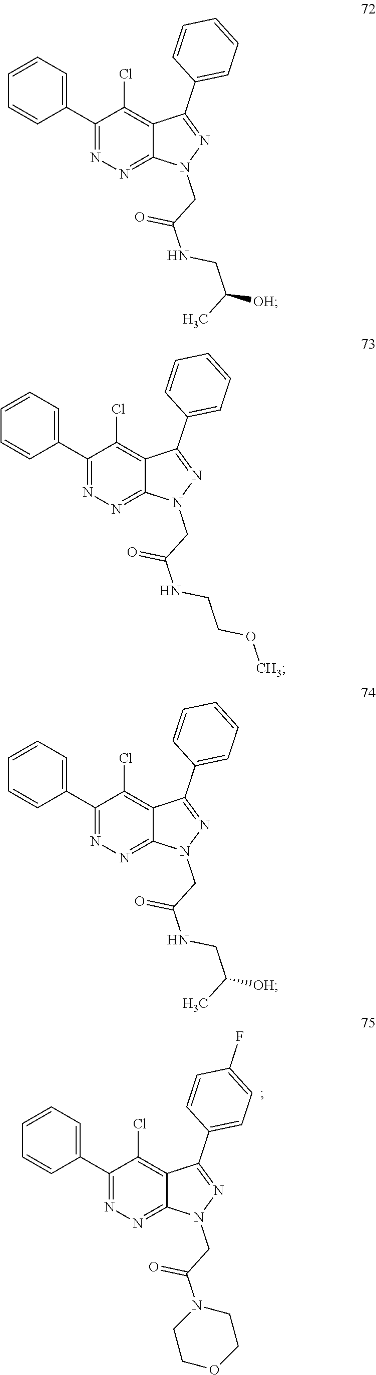 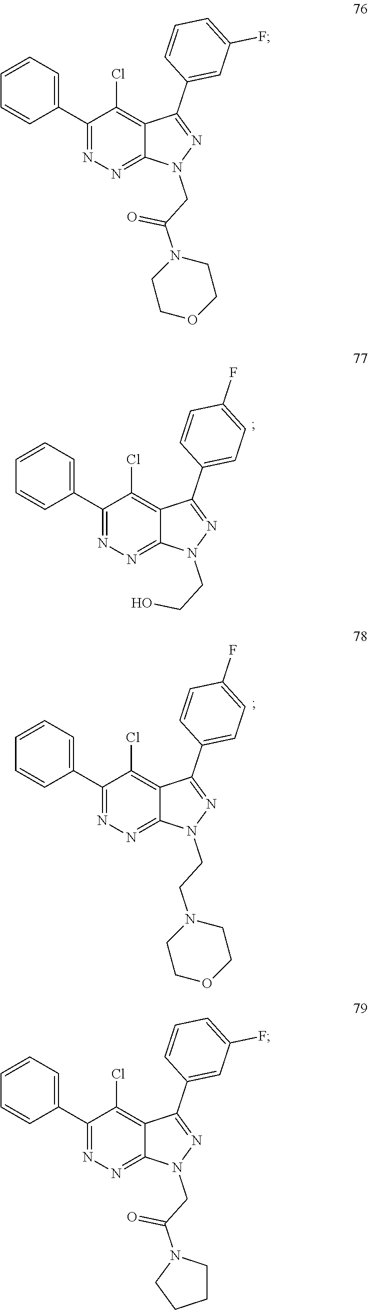 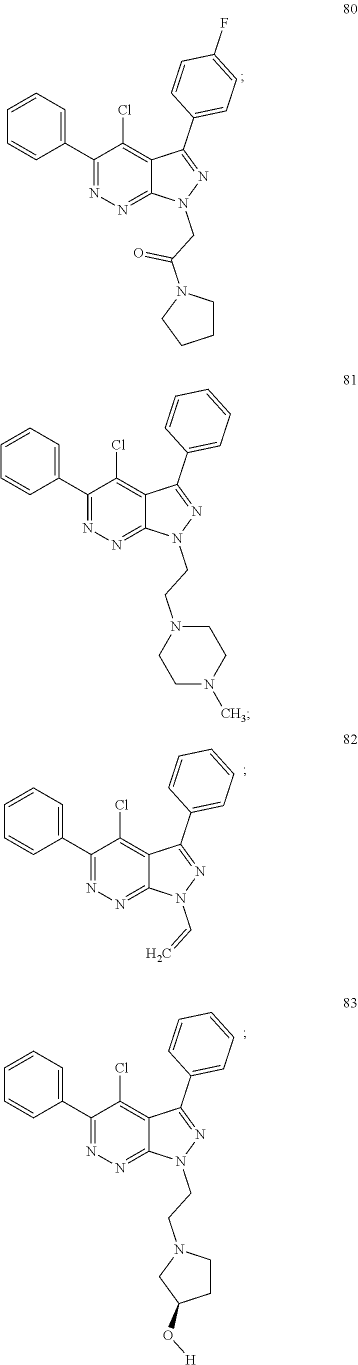 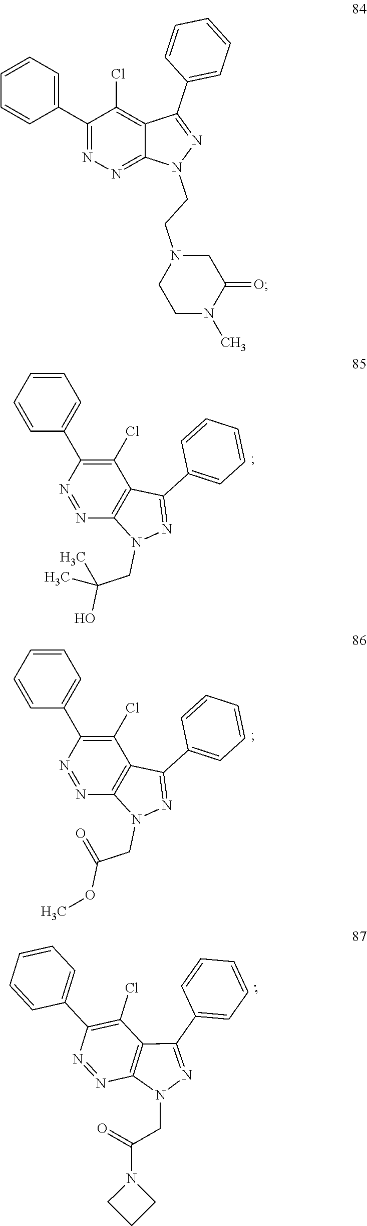 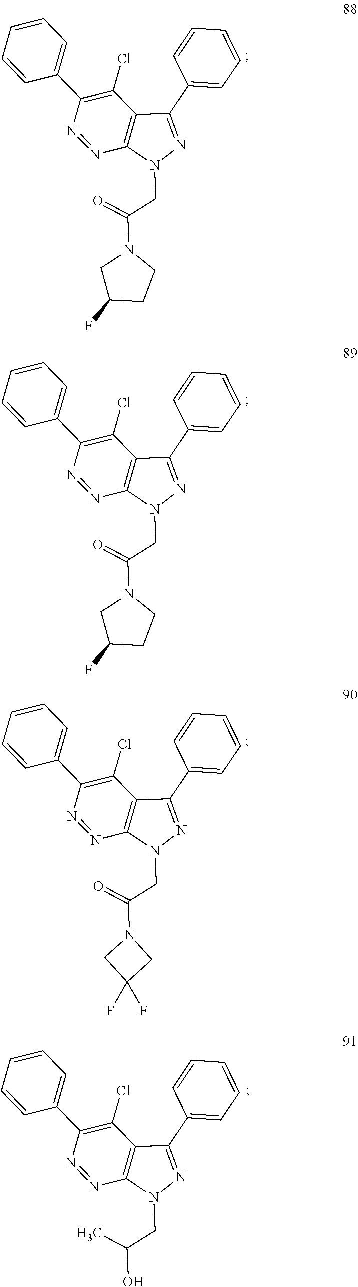 [SMILES: O=C(Cn1nc(-c2ccccc2)c2c(Cl)c(-c3ccccc3)nnc21)N1CC[C@@H](F)C1 ; O=C(Cn1nc(-c2ccccc2)c2c(Cl)c(-c3ccccc3)nnc21)N1CC[C@@H](F)C1 ; O=C(Cn1nc(-c2ccccc2)c2c(Cl)c(-c3ccccc3)nnc21)N1CC(F)(F)C1 ; CC(O)Cn1nc(-c2ccccc2)c2c(Cl)c(-c3ccccc3)nnc21] 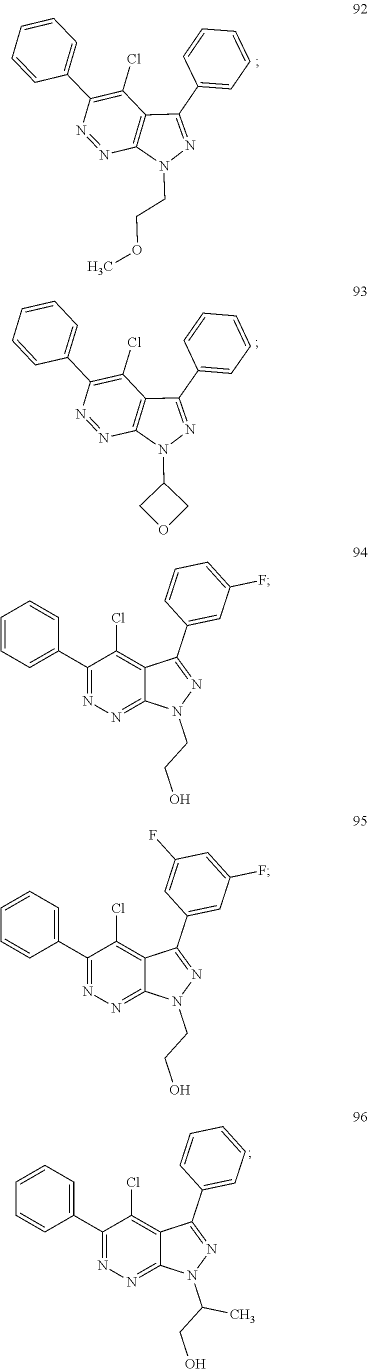 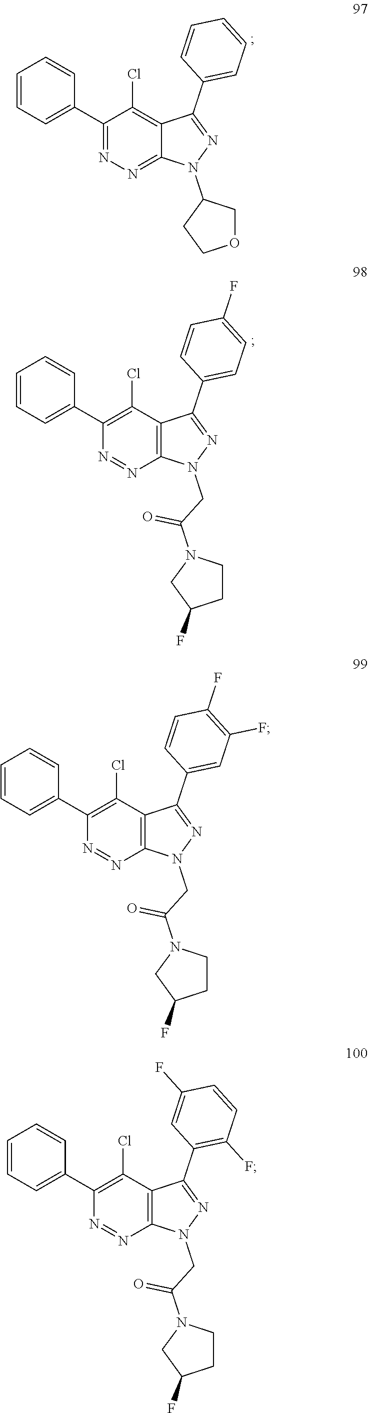 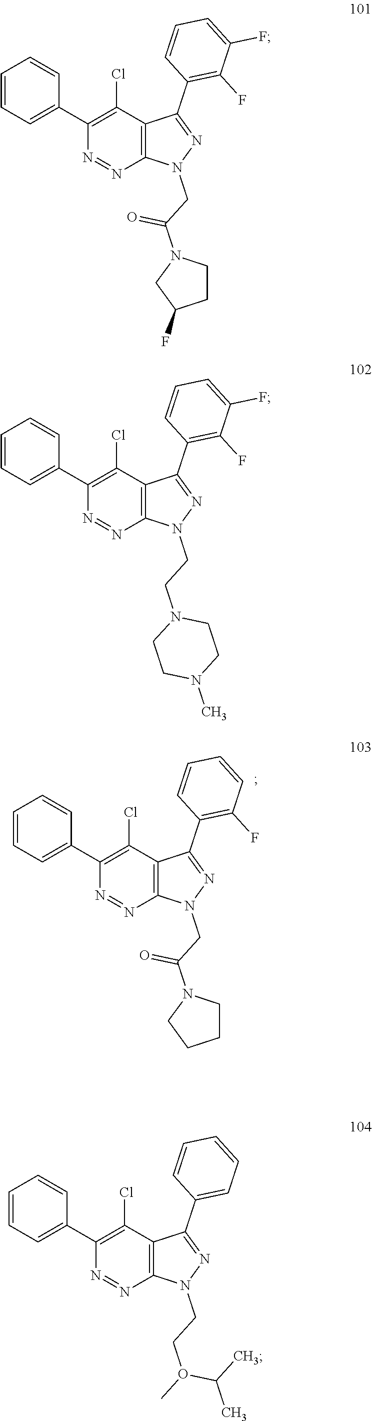 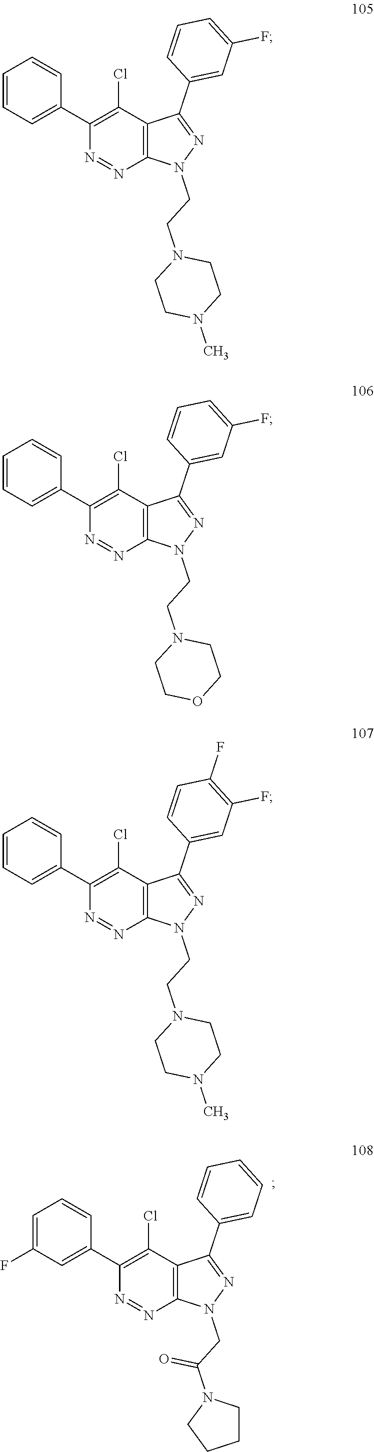 [SMILES: CN1CCN(CCn2nc(-c3cccc(F)c3)c3c(Cl)c(-c4ccccc4)nnc32)CC1 ; Fc1cccc(-c2nn(CCN3CCOCC3)c3nnc(-c4ccccc4)c(Cl)c23)c1 ; CN1CCN(CCn2nc(-c3ccc(F)c(F)c3)c3c(Cl)c(-c4ccccc4)nnc32)CC1 ; O=C(Cn1nc(-c2ccccc2)c2c(Cl)c(-c3cccc(F)c3)nnc21)N1CCCC1] 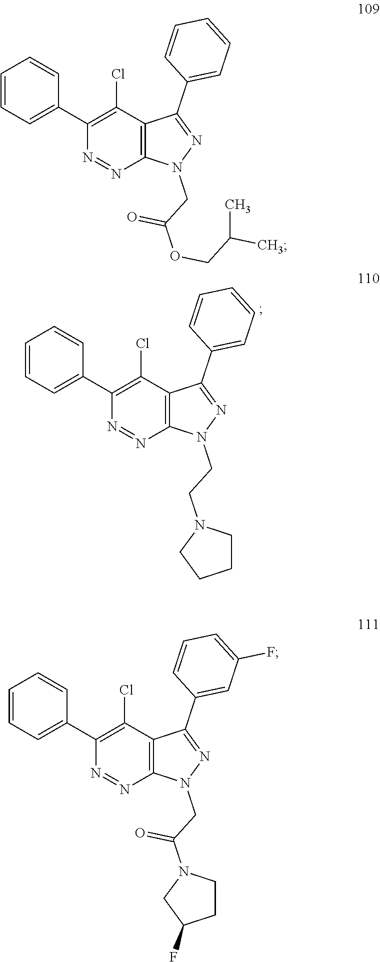
or a pharmaceutically acceptable salt thereof.

3.-23. (canceled)

24. The method of claim 1 or 2, wherein the proteopathy is a neurodegenerative disease.

25. The method of claim 24, wherein the neurodegenerative disease is Alzheimer's disease, progressive supranuclear palsy, dementia pugilistica, frontotemporal dementia and parkinsonism linked to chromosome 17 (FTDP-17), Lytico-Bodig disease, tangle-predominant dementia, ganglioma, gangliocytoma, meningioangiomatosis, subacute sclerosing panencephalitis, lead encephalopathy, tuberous sclerosis, Hallervorden-Spatz disease, lipofuscinosis, Pick's disease, corticobasal degeneration, argyrophilic grain disease, Huntington's disease, Parkinson's disease, dementia with Lewy bodies, multiple system atrophy, neuroaxonal dystrophies, dentatorubralpallidoluysian atrophy (DRPLA), spinal-bulbar muscular atrophy (SBMA), spinocerebellar ataxia 1 (SCA 1), SCA 2, SCA 3, SCA 6, SCA 7, SCA 17, prion disease, amyotrophic lateral sclerosis, frontotemporal lobar degeneration (FTLD) or familial encephalopathy with neuroserpin inclusion bodies (FENIB).

26. The method of claim 1 or 2, wherein the proteopathy is an amyloidosis.

27. The method of claim 26, wherein the amyloidosis is familial British dementia (ABri), familial Danish dementia (ADan), hereditary cerebral haemorrhage with amyloidosis-Icelandic (HCHWA-I), familial amyloidotic neuropathy (ATTR), AL (light chain) primary systemic amyloidosis, AH (heavy chain) amyloidosis, AA secondary amyloidosis, Aβ amyloidosis, aortic medial amyloidosis, LECT2 amyloidosis, AIAPP amyloidosis, apolipoprotein AI amyloidosis (AApoAI), apolipoprotein AII amyloidosis (AApoAII), apolipoprotein AIV amyloidosis (AApoAIV), familial amyloidosis of the Finnish type (FAF), fibrinogen amyloidosis (AFib), lysozyme amyloidosis (ALys), dialysis amyloidosis (Aβ₂M), medullary thyroid carcinoma (ACal), cardiac atrial amyloidosis (AANF), pituitary prolactinoma (APro), hereditary lattice corneal dystrophy, cutaneous lichen amyloidosis (AKer), Mallory bodies, primary cutaneous amyloidosis, corneal lactoferrin amyloidosis, odontogenic (Pindborg) tumor amyloid or seminal vesicle amyloid.

28. The method of claim 1 or 2, wherein the proteopathy is a lysosomal storage disease.

29. The method of claim 28, wherein the lysosomal storage disease is a mucopolysaccharidosis disorder.

30. The method of claim 29, wherein the mucopolysaccharidosis disorder is Pseudo-Hurler polydystrophy/Mucolipidosis IIIA, MPS I Hurler Syndrome, MPS I Scheie Syndrome, MPS I Hurler-Scheie Syndrome, MPS II Hunter syndrome, Sanfilippo syndrome Type A/MPS III A, Sanfilippo syndrome Type B/MPS III B, Sanfilippo syndrome Type C/MPS III C, Sanfilippo syndrome Type D/MPS III D, Morquio Type AMPS IVA, Morquio Type B/MPS IVB, MPS IX Hyaluronidase Deficiency, MPS VI Maroteaux-Lamy, MPS VII Sly Syndrome, Mucolipidosis I/Sialidosis, Mucolipidosis IIIC or Mucolipidosis type IV.

31. The method of claim 28, wherein the lysosomal storage disease is Pompe disease/glycogen storage disease type II.

32. The method of claim 28, wherein the lysosomal storage disease is activator deficiency/GM2 gangliosidosis, alpha-mannosidosis, aspartylglucosaminuria, cholesteryl ester storage disease,chronic Hexosaminidase A Deficiency, cystinosis, Danon disease, Fabry disease, Farber disease, fucosidosis, galactosialidosis, Gaucher Disease Type I, Gaucher Disease Type II, Gaucher Disease Type III, GM1 gangliosidosis infantile, GM1 gangliosidosis late infantile/juvenile, GM1 gangliosidosis adult/chronic, I-Cell disease/Mucolipidosis II, Infantile Free Sialic Acid Storage Disease/ISSD, Juvenile Hexosaminidase A Deficiency, Krabbe disease infantile onset, Krabbe disease late onset, lysosomal acid lipase deficiency early onset, lysosomal acid lipase deficiency Late onset, Metachromatic Leukodystrophy, Pseudo-Hurler polydystrophy/Mucolipidosis IIIA, MPS I Hurler Syndrome, MPS I Scheie Syndrome, MPS I Hurler-Scheie Syndrome, MPS II Hunter syndrome, Sanfilippo syndrome Type AMPS III A, Sanfilippo syndrome Type B/MPS III B, Sanfilippo syndrome Type C/MPS III C, Sanfilippo syndrome Type D/MPS III D, Morquio Type AMPS IVA, Morquio Type B/MPS IVB, MPS IX Hyaluronidase Deficiency, MPS VI Maroteaux-Lamy, MPS VII Sly Syndrome, Mucolipidosis I/Sialidosis, Mucolipidosis IIIC, Mucolipidosis type IV, Multiple sulfatase deficiency, Niemann-Pick Disease Type A, Niemann-Pick Disease Type B, Niemann-Pick Disease Type C, CLN6 disease-atypical late infantile, CLN6 disease-late onset variant, CLN6 disease-early juvenile, Batten-Spielmeyer-Vogt/Juvenile NCL/CLN3 disease, Finnish Variant Late Infantile CLNS, Jansky-Bielschowsky disease/Late infantile CLN2/TPP1 Disease, Kufs/Adult-onset NCL/CLN4 disease, Northern Epilepsy/variant late infantile CLN8, Santavuori-Haltia/Infantile CLN1/PPT disease, Beta-mannosidosis, Pompe disease/glycogen storage disease type II, Pycnodysostosis, Sandhoff disease/Adult Onset/GM2 Gangliosidosis, Sandhoff disease/GM2 gangliosidosis—Infantile, Sandhoff disease/GM2 gangliosidosis—Juvenile, Schindler disease, Salla disease/Sialic Acid Storage Disease, Tay-Sachs/GM2 gangliosidosis or Wolman disease.

33.-34. (canceled)
